(19) United States
(12) Patent Application Publication
(10) Pub. No.: US 2024/0216525 A1
(43) Pub. Date: Jul. 4, 2024

(54) ENZYME-TRIGGERED SELF-REACTING LINKER HAVING IMPROVED PHYSICOCHEMICAL AND PHARMACOLOGICAL PROPERTIES

(71) Applicants: MABLINK BIOSCIENCE, Lyon (FR); CENTRE NATIONAL DE LA RECHERCHE SCIENTIFIQUE - CNRS -, Paris (FR); UNIVERSITE CLAUDE BERNARD LYON 1, Villeurbanne (FR)

(72) Inventors: Warren VIRICEL, Grézieu La Varenne (FR); Benoît JOSEPH, Villeurbanne (FR); Guy FOURNET, Lyon (FR)

(73) Assignees: MABLINK BIOSCIENCE, Lyon (FR); CENTRE NATIONAL DE LA RECHERCHE SCIENTIFIQUE - CNRS -, Paris (FR); UNIVERSITE CLAUDE BERNARD LYON 1, Villeurbanne (FR)

(21) Appl. No.: 18/285,175

(22) PCT Filed: Mar. 30, 2022
(86) PCT No.: PCT/EP2022/058402

§ 371 Date: Sep. 29, 2023

Related U.S. Application Data
(60) Provisional application No. 63/167,915, filed on Mar. 30, 2021.

(30) Foreign Application Priority Data
Mar. 30, 2021 (EP) 21305405.9

(51) Int. Cl. A61K 47/68; A61P 35/00

(52) U.S. Cl. CPC A61K 47/6889; A61K 47/68037; A61K 47/6851; A61P 35/00

(57) ABSTRACT

Enzyme-triggered self-reacting arm compounds and chemical intermediates used for preparing such compounds and uses thereof, specifically in prodrug design and conjugation technologies. A Ligand-Drug-Conjugate (LDC) includes such enzyme-triggered self-reacting arms.

### TECHNICAL FIELD

The present invention pertains to compounds comprising an enzyme-triggered self-reacting arm, chemical intermediates used for preparing such compounds, and uses thereof, specifically in prodrug design and conjugation technologies.

The present invention also relates to a Ligand-Drug-Conjugate (LDC) comprising such enzyme-triggered self-reacting arm.

### BACKGROUND

Ligand-drug-conjugates (LDCs) are designed to specifically deliver active compounds to targeted tissues while sparing healthy tissues. For example, in the case of a LDC aiming to deliver a cytotoxic anticancer agent to tumor cells, the LDC format can be used to improve the toxicity profile and improve the tolerability of the treatment.

LDCs comprise at least one ligand unit, which is usually a polypeptide, a protein or a targeting small molecule, that is covalently linked to at least one therapeutic, diagnostic or labelling compound (hereinafter referred as drug or D) via a synthetic linker. This synthetic linker may comprise one or several mono- or di-valent arms for joining the ligand unit(s) and the drug unit(s), which may be selected from spacers, connectors and enzyme-sensitive cleavable moieties. Said linker may also orthogonally bear any moiety that can improve the LDC performance, such as storage stability, plasmatic stability or pharmacokinetics properties. When the ligand unit of the conjugate is an antibody or an antibody fragment and is associated with an immunostimulatory, cytotoxic or chemotherapy drug, the term Antibody-Drug-Conjugate (ADC) is commonly used.

When designing a LDC, there is a need to covalently attach the final active drug to the ligand targeting unit, while allowing the final release of the drug unit by a selective enzymatic mechanism after cellular internalization, or in the diseased tissue microenvironment. In this regard, several peptidase- and glycosidase-sensitive cleavable linker chemical strategies (associated with self-immolative chemistries) were developed. These cleavable linkers and their corresponding cleavage mechanisms are well known and have been described in several publications (e.g. Bargh J G et al., Chem. Soc. Rev., 2019, 48, 4361, Toki et al. J. Org. Chem. 2002, 67, 6, 1866-1872, Scott et al. Bioconjugate Chem. 2006, 17, 3, 831-840).

For example, WO2011145068 discloses the use of a glycosidase-sensitive cleavable drug-linker based on the 4-(1-hydroxybut-3-yn-1-yl)phenol self-immolative chemical spacer, thus conferring a terminal alkyne handle for click chemistry capabilities (cf. formula below).

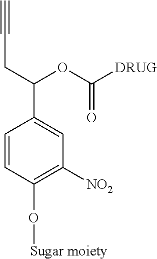

WO2017089895 describes the use of a glycosidase-sensitive cleavable drug-linker based on the 2-hydroxy-5-(hydroxymethyl)benzoic acid self-immolative chemical spacer (cf. formula below).

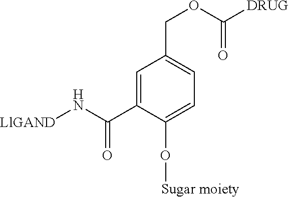

However, it is always desirable to develop alternative enzyme-sensitive self-immolative linkers that would improve the physicochemical and pharmacological properties of LDCs.

Thus, in this context, one of the objectives of the present disclosure is to provide an enzyme-sensitive self-immolative linker that can be used to prepare LDCs.

Another objective of the present disclosure is an enzyme-sensitive self-immolative linker that can improve the physicochemical and/or pharmacological properties of LDCs.

Another objective of the present disclosure is to provide compounds that can be used to prepare LDCs.

### SUMMARY

In a first aspect, the present disclosure relates to a Ligand-Drug-Conjugate compound (LDC) having the following formula (I)

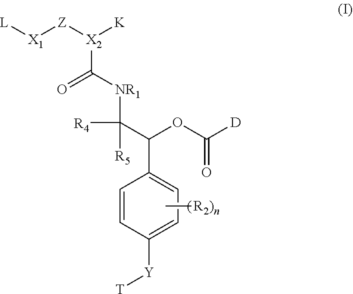

### Wherein

L is a ligand;
X1 is a connector unit;
Z is an optional spacer;
X2 is a connector unit;
K is an optional hydrophobicity masking entity, preferably selected from polysarcosine and polyethylene glycol;
R1 is selected from the group consisting of H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
D is an active agent, preferably selected from the group consisting of drugs, imaging agents and fluorophores;
each R2 is independently selected from the group consisting of electron-withdrawing groups and C₁-C₄ alkyl;
n is 0, 1 or 2;
R4 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
R5 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
T is a sugar cleavable unit or a polypeptide cleavable unit;
Y is O when T is a sugar cleavable unit, or NR3 when T is a polypeptide cleavable unit;
R3 is selected from the group consisting of H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R″ being independently selected from H and C₁-C₆ alkyl;
and pharmaceutically acceptable salts thereof.

It was surprisingly found that using a 2-amino-1-(4-aminophenyl)ethan-1-ol-based or a 2-amino-1-(4-oxophenyl)ethan-1-ol-based self-immolative linker, as in formula (I), yielded LDCs having improved hydrophilicity and improved in vivo pharmacokinetic profiles. This self-immolative linker also enables an easier manufacturing, purification and formulation of the compound of formula (I), and thus a higher yield. The compound of formula (I) also has less aggregation potential, due to the presence of this specific linker.

The present disclosure also relates to a pharmaceutical composition comprising a compound of the present disclosure, preferably a compound of formula (I) and a pharmaceutically acceptable carrier.

The present disclosure also relates to a compound of the present disclosure, preferably a compound of formula (I) for use as a drug.

The present disclosure also relates to an intermediate compound of formula (II):

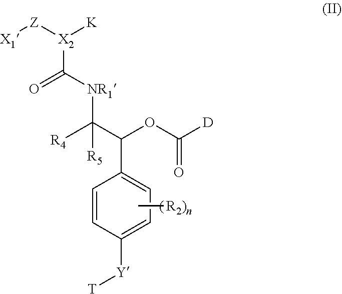

### Wherein

X1′ is a group which can react with a ligand to form a connector unit;
is an optional spacer;
X2 is a connector unit;
K is an optional hydrophobicity masking entity, preferably selected from polysarcosine and polyethylene glycol;
R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
D is an active agent, preferably selected from the group consisting of drugs, imaging agents and fluorophores;
each R2 is independently selected from the group consisting of electron-withdrawing groups and C₁-C₄ alkyl;
n is 0, 1 or 2;
R4 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
R5 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
T is a sugar cleavable unit or a polypeptide cleavable unit;
Y′ is O when T is a sugar cleavable unit, or NR3′ when T is a polypeptide cleavable unit;
R3′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R″ being independently selected from H and C₁-C₆ alkyl;
and pharmaceutically acceptable salts thereof.

The present disclosure also relates to an intermediate compound of formula (III)

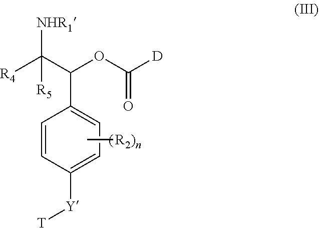

### Wherein

R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR″—, —NR″—C(O)—O—, —O—C(O)NR″— and triazole;
D is an active agent, preferably selected from the group consisting of drugs, imaging agents and fluorophores;
each R2 is independently selected from the group consisting of electron-withdrawing groups and C₁-C₄ alkyl;
n is 0, 1 or 2;
R4 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
R5 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
T is a sugar cleavable unit or a polypeptide cleavable unit;
Y′ is O when T is a sugar cleavable unit, or NR3′ when T is a polypeptide cleavable unit;
R3′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R′″ being independently selected from H and C₁-C₆ alkyl;
and pharmaceutically acceptable salts thereof.

The present disclosure also relates to an intermediate compound of formula (IV)

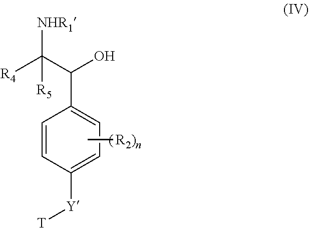

### Wherein

R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR″—, —NR″—C(O)—O—, —O—C(O)NR″— and triazole;
each R2 is independently selected from the group consisting of electron-withdrawing groups;
n is 0, 1 or 2;
R4 is selected from the group consisting of H, C₁-C₆ alkyl and C₂-C₆ alkenyl, said alkyl and alkenyl being optionally interrupted by one or more heteroatom or chemical groups selected from —O—, —S—, —C(O)—, and —NR″—;
R5 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
T is a sugar cleavable unit or a polypeptide cleavable unit;
Y′ is O when T is a sugar cleavable unit, or NR3′ when T is a polypeptide cleavable unit;
R3′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R″ being independently selected from H and C₁-C₆ alkyl;
and pharmaceutically acceptable salts thereof.

### BRIEF DESCRIPTION OF THE FIGURES

FIGS. 1 & 2 represent the hydrophobic interaction chromatograms according to example 3.

FIGS. 3 & 4 represent the in vitro cytotoxicity assays of conjugates according to example 4.

FIG. 5 represents the in vitro cytotoxicity assays of conjugates according to example 5.

FIGS. 6, 7, 8 & 9 represent in vivo pharmacokinetic profiles in rats and pharmacokinetic parameters according to example 6.

FIG. 10 represents tumor volumes over time in a mice xenograft model of gastric cancer, according to example 7.

### DETAILED DESCRIPTION

### Definitions

Various embodiments of the disclosure are described herein. It will be recognized that features specified in each embodiment may be combined with other specified features to provide further embodiments.

The present disclosure encompasses the compounds of the present disclosure, their tautomers, enantiomers, diastereomers, racemates or mixtures thereof, and their hydrates, esters, solvates or pharmaceutically acceptable salts.

Any formula given herein is also intended to represent unlabeled as well as isotopically labeled forms of the compounds, like deuterium labeled compounds or ¹⁴C-labeled compounds.

The terms “pharmaceutically acceptable salts” refer to salts that retain the biological effectiveness and properties of the compounds of this disclosure and, which typically are not biologically or otherwise undesirable. In many cases, the compounds of the disclosure are capable of forming acid and/or base salts by virtue of the presence of amino and/or carboxyl groups or groups similar thereto. Pharmaceutically acceptable acid addition salts can be formed with organic acids and/or inorganic acids. Pharmaceutically acceptable base addition salts can be formed with organic bases and/or inorganic bases. Such salts are well-known from those skilled in the art

As used herein, the terms “C₁-C₂₄ alkyl”, by itself or as part of another substituent, refer to a linear or branched alkyl functional group having 1 to 24 carbon atoms, preferably 1 to 20 carbon atoms, more preferably 1 to 12 or 1 to 6 carbon atoms. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

Alkylene, used alone or as part of alkylene glycol for example, refers to a divalent saturated, straight-chained or branched alkyl group as defined herein.

Alkenyl and alkynyl refer to at least partially unsaturated, straight-chained or branched hydrocarbon group having 2-20 carbon atoms, preferably 2-12, more preferably 2-6, especially 2-4. An alkenyl group comprises at least one C═C double bond; an alkynyl group comprises at least one C═C triple bond.

As used herein, the terms “C₃-C₈ cycloalkyl” or “carbocycle” refer to a saturated or unsaturated cyclic group having 3 to 8 carbon atoms, preferably 3 to 6. The cycloalkyl can have a single ring or multiple rings fused together. The cycloalkyl can also include spirocyclic rings. Suitable cycloalkyl groups include cyclopropyl, cyclobutyl, cyclopentyl and cyclohexyl.

As used herein, the terms “C₃-C₈ cycloalkylene” or “carbocyclo” refer to a divalent cycloalkyl as defined herein.

As used herein, the term “halogen” refers to a fluoro (—F), chloro (—Cl), bromo (—Br), or iodo (—I) group.

As used herein, the terms “C₁-C₆ haloalkyl” refer to a C₁-C₆ alkyl as defined herein that is substituted by one or more halogen group as defined herein. Suitable C₁-C₆ haloalkyl groups include trifluoromethyl and dichloromethyl.

As used herein, the term “heteroalkyl”, refers to a straight or branched hydrocarbon chain consisting of 1 to 12 carbon atoms, preferably 1 to 10, more preferably 1 to 6 carbon atoms, and from one to three heteroatoms selected from the group consisting of O, N, Si and S, and wherein the nitrogen and sulfur atoms may optionally be oxidized (for example: a sulfoxide or a sulfone) and the nitrogen heteroatom may optionally be quaternized. The heteroatom(s) O, N and S may be placed at any interior position of the heteroalkyl group or at the position at which the alkyl group is attached to the remainder of the molecule.

Heteroalkylene refers to a divalent heteroalkyl as defined above. For heteroalkylene groups, heteroatoms can also occupy either or both of the chain termini.

As used herein, the terms “C₁-C₆ alkoxy” refer to a —O-alkyl group, wherein the alkyl group is a C₁-C₆ alkyl as defined herein. Suitable C₁-C₆ alkoxy groups include methoxy, ethoxy, propoxy.

As used herein, the terms “C₁-C₆ haloalkoxy” refer to a C₁-C₆ alkoxy group as defined herein, that is substituted by one or more halogen group as defined herein. Suitable haloalkoxy include trifluoromethoxy.

As used herein, the terms “aryl having 6 to 10 ring atoms” refer to a polyunsaturated, aromatic hydrocarbyl group having a single ring or multiple aromatic rings fused together, containing 6 to 10 ring atoms, wherein at least one ring is aromatic. The aromatic ring may optionally include one to two additional rings (cycloalkyl, heterocyclyl or heteroaryl as defined herein) fused thereto. Suitable aryl groups include phenyl, naphthyl and phenyl ring fused to a heterocyclyl, like benzopyranyl, benzodioxolyl, benzodioxanyl and the like.

Arylene refers to a divalent aryl group as defined above.

As used herein, the terms “heteroaryl having 5 to 10 ring atoms” refer to a polyunsaturated, aromatic ring system having a single ring or multiple aromatic rings fused together or linked covalently, containing 5 to 10 atoms, wherein at least one ring is aromatic and at least one ring atom is a heteroatom selected from N, O and S. The nitrogen and sulfur heteroatoms may optionally be oxidized and the nitrogen heteroatoms may optionally be quaternized. Such rings may be fused to an aryl, cycloalkyl or heterocyclyl ring. Non-limiting examples of such heteroaryl, include: furanyl, thiophenyl, pyrrolyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, indolyl, isoindolyl, benzofuranyl, isobenzofuranyl, benzothiophenyl, isobenzothiophenyl, indazolyl, benzimidazolyl, benzoxazolyl, purinyl, benzothiadiazolyl, quinolinyl, isoquinolinyl, cinnolinyl, quinazolinyl and quinoxalinyl.

As used herein, the terms “heterocyclyl having 3 to 10 ring atoms”, “heterocycloalkyl having 3 to 10 ring atoms” or “heterocyclyl” refer to a saturated or unsaturated cyclic group having 3 to 10 ring atoms, preferably 3 to 8 ring atoms, wherein at least one ring atom is a heteroatom selected from N, O and S. The nitrogen and sulfur heteroatoms may optionally be oxidized and the nitrogen heteroatoms may optionally be quaternized. The heterocycle can include fused or bridged rings as well as spirocyclic rings. Examples of heterocycle include, but are not limited to, tetrahydropyridyl, piperidinyl, morpholinyl, tetrahydrofuranyl, tetrahydrothienyl, piperazinyl, 1-azepanyl, imidazolinyl, 1,4-dioxanyl and the like.

As used herein, the terms “heterocyclo” or “heterocycloalkylene” refer to a divalent heterocycle as defined herein.

Furthermore, the terms alkyl, alkenyl, alkynyl, aryl, alkylene, arylene, heteroalkyl, heteroalkylene, C₃-C₈ carbocycle, C₃-C₈ carbocyclo, C₃-C₈ heterocycle, C₃-C₈ heterocyclo, polyether refer to optionally substituted groups with one or more of the substituents selected from: —X, —R′, —O, —OR′, ═O, —SR′, —S, —NR′₂, —NR′₃, ═NR′, —CX₃, —CN, —OCN, —SCN, —N═C—O, —NCS, —NO, —NO₂, ═N₂, —N₃, —NRC(═O)R′, —C(═O)R′, —C(═O)NR′₂, —SO₃⁻, —SO₃H, —S(═O)₂R′, —OS(═O)₂OR′, —S(═O)₂NR′, —S(═O)R′, —OP(═O)(OR′)₂, —P(═O)(OR′)₂, —PO₃⁻, —PO₃H₂, —C(═O)R′, —C(═O)X, —C(═S)R′, —CO₂R′, —CO₂, —C(═S)OR′, C(═O)SR′, C(═S)SR′, C(═O)NR′2, C(═S)NR′₂, and C(═NR′)NR′₂, where each X is independently a halogen: —F, —Cl, —Br, or —I; and each R′ is independently —H, —C₁-C₂₀ alkyl, —C₆-C₁₀ aryl, or —C₃-C₁₀ heterocycle.

Acyl group refers to —CO-alkyl wherein alkyl has the definition above.

As used herein, the term “polyether” refers to a polymer containing ether linkage. The number of ether moieties in the polyether may be comprised between 2 and 100, preferably between 2 and 25, in particular between 2 and 10. Examples of polyether include polyethylene glycol, like polyethylene glycol having between 2 and 100 ether moieties, preferably between 2 and 25, and in particular between 2 and 10.

The terms “optionally substituted polyether” can refer to a polyether, and in particular a polyethylene glycol, that is optionally substituted with one or more of the substituents selected from: halogen, oxo, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″, —NR″—(CO)—R′, and —NR″R″; R′, R″ and R″ being independently selected from H and C₁-C₆ alkyl.

Solid-phase peptide synthesis (SPPS) refers to a well-known process in which a peptide anchored to a support, an insoluble polymer, is assembled by the successive addition of Fmoc- or Boc-protected aminoacids, via repeated cycles of deprotection-wash-coupling-wash. Each aminoacid addition is referred to as a cycle of: (i) cleavage of the Nα-protecting group, (ii) washing steps, (iii) coupling of a fluroenylmethoxycarbonyl- (Fmoc-) or tert-butyloxycarbonyl- (Boc-) protected aminoacid using coupling reagents and a non-nucleophilic base, (iv) washing steps. As the growing chain is bound to said support the excess of reagents and soluble by-products can be removed by simple filtration. Because repeated coupling reactions with hindered Fmoc- or Boc-protected N-methylated aminoacids are difficult and often suboptimal, low crude purity, difficult purification and low yields are to be expected with this technique. Examples of said support are Wang resin, Rink amide resin, trityl- and 2-chlorotrityl resins, PAM resin, PAL resin, Sieber amide resin, MBHA resin, HMPB resin, HMBA resin which are commercially available and on which the peptide is directly or indirectly bound.

A ligand drug conjugate (LDC) refers to any conjugate that covalently connects a ligand and a drug as defined herein and involving any mean such as described herein, and that will be illustrated in the examples of the description. When the ligand is an antibody, one may refer to antibody drug conjugate (ADC) which is a preferred embodiment of the present disclosure.

The term connector unit refers to a component that connects different parts of the compound together, for example, the connector can connect the ligand to a spacer, or a spacer to the amide function —CO—NR1-. The connector is a scaffold bearing attachment sites for components of the ligand-drug-conjugate, namely the ligand, the spacer, the hydrophobicity masking entity, and/or the amide function —CO—NR1-.

From his knowledge, the one skilled in the art is capable to select a connector which is appropriate to the expected LDC compound. Non-exhaustive listing of connectors includes: aminoacids, for example lysine, glutamic acid, aspartic acid, serine, tyrosine, cysteine, selenocysteine, glycine, homoalanine; amino alcohols; amino aldehydes; polyamines or any combination thereof. Advantageously, the connector unit X1 and/or X2 is one or more natural or non-natural aminoacids. In one embodiment, the connector unit X1 and/or X2 is selected from glutamic acid, lysine and glycine. The connector units X1 and X2 can be independently selected from the group consisting of one or more amino acid(s), one or more N-substituted amino acid, optionally substituted polyether, C₁-C₁₂ alkylene, arylene having 6 to 10 ring atoms, C₃-C₈ cycloalkylene, heterocycloalkylene having 5 to 10 ring atoms, heteroarylene having 5 to 10 ring atoms, C₂-C₁₀ alkenylene, and any combination thereof, said alkylene and alkenylene being optionally interrupted by one or more heteroatoms or chemical groups selected from —O—, —S—, —C(O)—, —NR″—, —C(O)NR″—, —NR″—C(O)—, —NR″—C(O)—NR″—, —NR″—C(O)—O—, —O—C(O)NR″— and triazole,

and said alkylene, arylene, cycloalkylene, heterocycloalkylene, heteroarylene, and alkenylene being optionally substituted with one or more of the substituents selected from: halogen, oxo, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″, —NR″—(CO)—R′, and —NR″R′″; R′, R″ and R″ being independently selected from H and C₁-C₆ alkyl.

Examples of connector units include optionally substituted polyether, aminoacids, benzyl groups, amines, ketones,

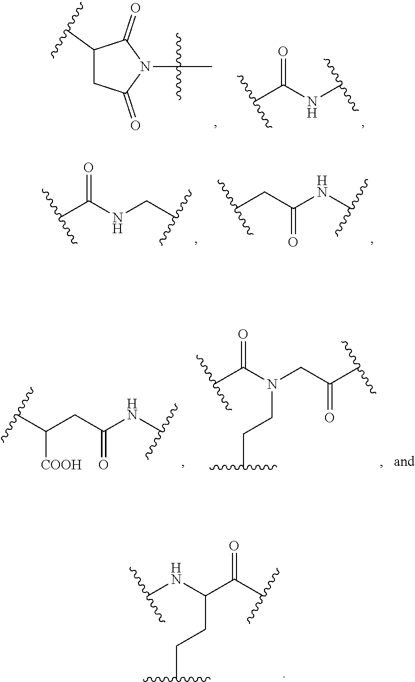

In particular, the connector unit can be divalent or trivalent. For example, X2 can be a trivalent connector unit when the hydrophobicity masking entity K is present.

The term “aminoacids” refers to natural or non-natural aminoacids. The CO moiety of the —CONR1- or —CONR1′- group can be considered as part of the X2 connector unit when X2 consists of one or more aminoacids. Non-exhaustive listing of aminoacids includes lysine, glutamic acid, aspartic acid, serine, tyrosine, cysteine, selenocysteine, glycine, and homoalanine.

A spacer is a divalent arm that covalently binds two components of the ligand-drug-conjugate, such as the 2 connector units. The spacer Z can be present or absent.

Non-exhaustive listing of spacer units includes: alkylene, heteroalkylene (so an alkylene interrupted by at least one heteroatom selected from Si, N, O and S); alkoxy; polyether such as polyalkylene glycol and typically polyethylene glycol; one or more natural or non-natural aminoacids such as glycine, alanine, proline, valine, N-methylglycine; C₃-C₈ heterocyclo; C₃-C₈ carbocyclo; arylene, and any combination thereof. For example, a spacer is a divalent linear alkylene group, preferably (CH₂)₄.

For example, the spacer can be selected from the group consisting of —C₁-C₁₀ alkylene-, —C₁-C₁₀ heteroalkylene-, —C₃-C₈ carbocyclo-, —O—(C₁ C₈ alkyl)-, -arylene-, —C₁-C₁₀ alkylene-arylene-,-arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀ heteroalkylene-C(═O)—, —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈ carbocyclo-S—, —O—(C₁-C₈ alkyl)-)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, —(C₃-C₈ heterocyclo)-C₁-C₁₀ alkylene-S—, —C₁-C₁₀ alkylene-O—C(═O)—, —C₃-C₈ carbocyclo-O—C(═O)—, —O—(C₁-C₈ alkyl)-O—C(═O)—, -arylene-O—C(═O)—, —C₁-C₁₀ alkylene-arylene-O—C(═O)—, -arylene-C₁-C₁₀ alkylene-O—C(═O)—, —C₁-C₁₀ alkylene-(C₃-C₈ carbocyclo)-O—C(═O)—, —(C₃-C₈ carbocyclo)-C₁-C₁₀ alkylene-O—C(═O)—, —C₃-C₈ heterocyclo-O—C(═O)—, —C₁-C₁₀ alkylene-(C₃-C₈heterocyclo)-O—C(═O)—, and —(C₃-C₈ heterocyclo)-C₁-C₁₀ alkylene-O—C(═O)—.

Any of the groups mentioned above is optionally substituted with one or more of the substituents selected from: —X, —R′, —O′, —OR′, ═O, —SR′, —S, —NR′₂, —NR′₃+, ═NR′, —CX₃, —CN, —OCN, —SCN, —N═C═O, —NCS, —NO, —NO₂, ═N₂, —N₃, —NR′C(═O)R′, —C(═O)R′, —C(═O)NR′₂, —SO₃, —SO₃H, —S(═O)₂R′, —OS(═O)₂OR′, —S(═O)₂NR′, —S(═O)R′, —OP(═O)(OR′)₂, —P(═O)(OR′)₂, —PO₃, —PO₃H₂, —C(═O)X, —C(═S)R′, —CO₂R′, —CO₂, —C(═S)OR′, C(═O)SR′, C(═S)SR′, C(═O)NR′₂, C(═S)NR′₂, and C(═NR′)NR′₂, where each X is independently a halogen: —F, —Cl, —Br, or —I; and each R′ is independently —H, —C₁-C₂₀ alkyl, —C₆-C₁₀ aryl, or —C₃-C₁₀ heterocycle.

A ligand refers to any macromolecule (polypeptide, protein, peptides, typically antibodies) as usually employed in LDC (e.g. Antibody-Drug Conjugates) technologies, or to a small-molecule such as folic acid or an aptamer, that may be covalently conjugated with synthetic linkers or drug-linkers of the present work, using bioconjugation techniques (see Greg T. Hermanson, Bioconjugate Techniques, 3rd Edition, 2013, Academic Press). The ligand is traditionally a compound that is selected for its targeting capabilities. Non-exhaustive listing of ligands includes: proteins, polypeptides, peptides, antibodies, full-length antibodies and antigen-binding fragments thereof, interferons, lymphokines, hormones, growth factors, vitamins, transferrin or any other cell-binding molecule or substance. The main class of ligands used to prepare conjugates is antibodies. An example of protein is human serum albumin.

The term “antibody” as used herein is used in the broadest sense and covers monoclonal antibodies, polyclonal antibodies, modified monoclonal and polyclonal antibodies, monospecific antibodies, multispecific antibodies such as bispecific antibodies, antibody fragments and antibody mimetics (Affibody®, Affilin®, Affimer®, Nanofitin®, Cell Penetrating Alphabody®, Anticalin®, Avimer®, Fynomer®, Monobodies or nanoCLAMP®). An example of an antibody is trastuzumab.

The term “antibody” as referred to herein includes whole antibodies and any antigen-binding fragments (i.e., “antigen-binding portion”) or single chains thereof.

A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The terms “antigen-binding portion” of an antibody (or simply “antigen portion”), as used herein, refers to full length or one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR), or any fusion proteins comprising such antigen-binding portion.

Furthermore, although the two domains of the Fv fragment, V_{L} and V_{H}, are coded for by separate genes, they can be joined, using recombinant methods, by a synthetic linker that enables them to be made as a single chain protein in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

In specific embodiments, the ligand of the LDC is a chimeric, humanized or human antibody.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutant versions of human germline sequences or antibody containing consensus framework sequences derived from human framework sequences analysis, for example, as described in Knappik, et al. (2000. J Mol Biol 296, 57-86).

The human antibodies may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences.

As used herein, “isotype” refers to the antibody class (e.g., IgM, IgE, IgG such as IgG1 or IgG4) that is provided by the heavy chain constant region genes.

The phrases “an antibody recognizing an antigen” and “an antibody specific for an antigen” are used interchangeably herein with the term “an antibody which binds specifically to an antigen”.

An active agent refers to bioactive molecule or a therapeutic molecule. Examples of active agents include drugs, imaging agents and fluorophores. Among imaging agents, one can cite fluorophores such as indocyanine green.

A drug refers to any type of drug or compounds having intrinsic pharmacological or diagnostic properties, for example cytotoxic, cytostatic, immunomodulator, immunosuppressive, immunostimulant, anti-inflammatory, ionizing or anti-infective compounds. Among cytotoxic compounds, one can cite calicheamicins; uncialamycins; auristatins (such as monomethyl auristatin E known as MMAE); halichondrin derivatives (such as eribulin), tubulysin analogs; maytansines; cryptophycins; benzodiazepine dimers (including Pyrrolo[2,1-c][1,4]benzodiazepines known as PBD's); indolinobenzodiazepines pseudodimers (IGNs); duocarmycins; anthracyclins (such as doxorubicin or PNU159682); camptothecin analogs (such as 7-Ethyl-10-hydroxy-camptothecin known as SN38 or exatecan); Bcl2 and Bcl-xl inhibitors; thailanstatins; amatoxins (including α-amanitin); kinesin spindle protein (KSP) inhibitors; vinorelbine; cyclin-dependent kinase (CDK) inhibitors; molecular glue degraders, bleomycin; dactinomycin or radionuclides and their complexing agent (such as DOTA/¹⁷⁷Lu). Among anti-inflammatory drugs, one can cite corticosteroids such as dexamethasone or fluticasone. Among anti-infective drugs, one can cite antibiotics such as rifampicin or vancomycin. The drug can be an anticancer drug or an immunomodulator. Examples of anticancer drugs are cytotoxic and cytostatic compounds, preferably cytotoxic compounds.

A hydrophobicity masking entity refers to a group that can reduce the apparent hydrophobicity of the compound. The hydrophobicity masking entity can be selected from polysarcosine, polyethylene glycol, and chitooligosaccharide. The number of ethylene glycol or sarcosine moieties may vary in a wide range. For instance, the number of ethylene glycol or sarcosine moieties in the hydrophobicity masking entity may be comprised between 2 and 500, preferably between 5 and 100, in particular between 5 and 25. In an embodiment, the hydrophobicity masking entity is a polysarcosine comprising from 6 to 24 sarcosine moieties, preferably comprising from 10 to 12 sarcosine moieties. The number of chitosan in chitooligosaccharide can be comprised between 2 and 20, for example between 2 and 8.

An electron withdrawing group refers to an atom or group that draws electron density from neighboring atoms towards itself, usually by resonance or inductive effect. Electron withdrawing groups include halogens, haloalkyl (like —CF₃), —CN, —SO₃H, —NO₂, and —C(O)R groups, with R═H, OH, or alkoxy. Advantageously, the electron withdrawing group is —NO₂. In an embodiment, the electron-withdrawing group is in ortho position with regard to the Y-T substituent of the phenyl ring.

As used herein, the terms “protecting group” refer to a chemical substituent which can be selectively removed by readily available reagents which do not attack the regenerated functional group or other functional groups in the molecule. Suitable protecting groups are known in the art and continue to be developed. Suitable protecting groups may be found, for example in Wutz et al. (“Greene's Protective Groups in Organic Synthesis, Fourth Edition,” Wiley-Interscience, 2007). Protecting group for protection of the amino group as described by Wutz et al. (pages 696-927), are used in certain embodiments. Representative examples of amino protecting groups include, but are not limited to, t-butyloxycarbonyl (Boc), 9-fluorenyl methoxycarbonyl (Fmoc), Acetyl (Ac), carboxybenzyl group (Cbz), benzyl group (Bn), allyl, trifluoroacetyl, allyloxy carbonyl (Alloc) group and 2,2,2-trichloroethoxycarbonyl (Troc).

A group that can react with a ligand to form a connector unit refers to any chemical moiety that is being reactive for covalently binding a ligand. In particular, it may react with a thiol group present on the ligand.

Non-exhaustive listing of chemical moieties that are being reactive for covalently binding a ligand includes: carboxylic acid; primary amine; secondary amine; tertiary amine; hydroxyl; halogen; activated ester such as N-hydroxysuccinimide ester, perfluorinated esters, nitrophenyl esters, aza-benzotriazole and benzotriazole activated ester, acylureas; alkynyl; alkenyl; azide; isocyanate; isothiocyanate; aldehyde; thiol-reactive moieties such as maleimide, halomaleimides, haloacetyls, pyridyl disulfides; thiol; acrylate; mesylate; tosylate; triflate, hydroxylamine; chlorosulfonyl; boronic acid —B(OR′)₂ derivatives wherein R′ is hydrogen or alkyl group.

A “cleavable unit” refers to a chemical group that may be cleaved by action of an internal or external, preferably external, stimulus. In the present disclosure, the cleavable unit is either a polypeptide cleavable unit or a sugar cleavable unit. The stimulus triggering the cleavage of the cleavable unit may be for instance pH or temperature conditions, or the presence of an enzyme. Cleavage of the cleavable unit preferably triggers self-immolation of the phenyl-comprising linker of the compounds of the invention, and release of the active agent D. In the present disclosure, a “cleavable sugar unit” can refer to a sugar moiety, preferably a glucuronide or a galactoside. In the present disclosure, a “polypeptide cleavable unit” can refer to a polypeptide, preferably a dipeptide or a tripeptide.

As used herein, the terms “one or more” can be understood as referring to a number between 1 and 20, or between 1 and 10, or between 1 and 5.

### Compound of Formula (I)

The present disclosure relates to a compound of formula (I):

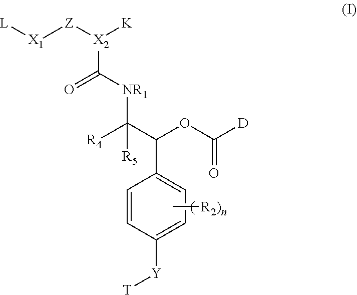

### Wherein

L is a ligand;
X1 is a connector unit;
Z is an optional spacer;
X2 is a connector unit;
K is an optional hydrophobicity masking entity, preferably selected from polysarcosine and polyethylene glycol;
R1 is selected from the group consisting of H, C₁-C₁₂ alkyl, C₂-C₆ alkenyl; optionally substituted polyether, polysarcosine, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
D is an active agent, preferably selected from the group consisting of drugs, imaging agents and fluorophores;
each R2 is independently selected from the group consisting of electron-withdrawing groups and C₁-C₄ alkyl;
n is 0, 1 or 2;
R4 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
R5 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
T is a sugar cleavable unit or a polypeptide cleavable unit;
Y is O when T is a sugar cleavable unit, or NR3 when T is a polypeptide cleavable unit;
R3 is selected from the group consisting of H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R′″ being independently selected from H and C₁-C₆ alkyl;
and pharmaceutically acceptable salts thereof.

According to an embodiment, L is a ligand selected from the group consisting of polypeptides, proteins, antibodies and antigen-binding fragments thereof, preferably L is an antibody or an antigen-binding fragment thereof, more preferably L is an antibody. According to an embodiment, the antibody is trastuzumab.

According to an embodiment, D is selected from the group consisting of drugs, preferably D is an anticancer drug or an immunomodulator. According to an embodiment, D is exatecan or monomethyl auristatin E (MMAE).

According to an embodiment, X1 and X2 are independently selected from the group consisting of one or more amino acid(s), one or more N-substituted amino acid(s), optionally substituted polyether, C₁-C₁₂ alkylene, arylene having 6 to 10 ring atoms, C₃-C₈ cycloalkylene, heterocycloalkylene having 5 to 10 ring atoms, heteroarylene having 5 to 10 ring atoms, C₂-C₁₀ alkenylene, and any combination thereof,
said alkylene and alkenylene being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole,
and said alkylene, arylene, cycloalkylene, heterocycloalkylene, heteroarylene, and alkenylene being optionally substituted with one or more of the substituents selected from: halogen, oxo, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″, —NR″—(CO)—R′, and —NR″R″;
R′, R″ and R′″ being independently selected from H and C₁-C₆ alkyl.

According to an embodiment, X1 is selected from the group consisting of one or more amino acid(s), one or more N-substituted amino acid(s), optionally substituted polyether, C₁-C₁₂ alkylene, arylene having 6 to 10 ring atoms and any combination thereof.

According to an embodiment, X1 is

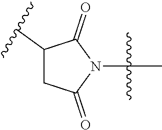 [SMILES: *C1CC(=O)N(*)C1=O]

According to an embodiment, X2 is selected from the group consisting of one or more amino acid(s), one or more N-substituted amino acid(s), C₁-C₁₂ alkylene, optionally substituted polyether and any combination thereof.

According to an embodiment, Z is selected from the group consisting of one or more amino acid(s), one or more N-substituted amino acid, optionally substituted polyether, C₁-C₁₂ alkylene, arylene having 6 to 10 ring atoms, C₃-C₈ cycloalkylene, heterocycloalkylene having 5 to 10 ring atoms, heteroarylene having 5 to 10 ring atoms, C₂-C₁₀ alkenylene, and any combination thereof,
said alkylene and alkenylene being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole, and said alkylene, arylene, cycloalkylene, heterocycloalkylene, heteroarylene, and alkenylene being optionally substituted with one or more of the substituents selected from: halogen, oxo, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R″, —NR″—(CO)—R′, and —NR″R″;
R′, R″ and R″ being independently selected from H and C₁-C₆ alkyl.

According to an embodiment, Z is selected from the group consisting of one or more amino acid(s), one or more N-substituted amino acid(s), and a polyethylene glycol.

According to an embodiment, Z is not present, and X1 and X2 are directly linked to each other through a single bond.

According to an embodiment, K is a polysarcosine, preferably a monodispersed polysarcosine. The polysarcosine can have from 1 to 50 repeatable units.

In specific embodiments, K is a polysarcosine, preferably of the following formula (V)

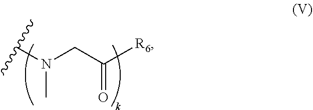

wherein k is an integer between 2 and 50, preferably between 4 and 30, and R6 corresponds to OH or NH2.

According to an embodiment, T is a sugar cleavable unit which is a glucuronide or a galactoside. The glycosidic bond linking T to the aryl group can be one that can be cleaved to initiate a self-immolative reaction sequence that leads to a release of the drug.

According to an embodiment, T is a dipeptide, preferably selected from Val-Cit, Val-Ala and Phe-Lys.

According to an embodiment, R1 is selected from the group consisting of H and C₁-C₆ alkyl, preferably H.

According to an embodiment, R3 is selected from the group consisting of H and C₁-C₆ alkyl, preferably H.

According to an embodiment, R4 is selected from the group consisting of H and C₁-C₆ alkyl.

According to an embodiment, R5 is selected from the group consisting of H and C₁-C₆ alkyl.

According to an embodiment, both R4 and R5 are H.

According to an embodiment n is 0, Y is NR3, and T is a polypeptide cleavable unit. According to another embodiment, n is 1, R2 is —NO₂, Y is O, and T is a sugar cleavable unit.

According to a preferred embodiment, L is an antibody or an antigen-binding fragment thereof.

According to an embodiment, the antibody or antigen-binding fragment thereof binds an antigen selected from the group comprising or consisting of CD19, CD20, CD22, CD30, CD37, CD79b, HER2, and PSMA.

According to an embodiment, the antibody or antigen-binding fragment thereof binds to HER2/neu.

Some examples of antibodies suitable as ligand L in the compound of formula (I) described herein include, but are not limited to, trastuzumab, brentuximab, loncastuximab, rosopatamab, rituximab, pinatuzumab, polatuzumab, and naratuximab.

According to an embodiment, the antibody is trastuzumab.

The compound of formula (I) has at least one asymmetric carbon, so there can be different stereoisomers. For example, the compound of formula (I) can exist on a (R) form or a (S) form as shown below:

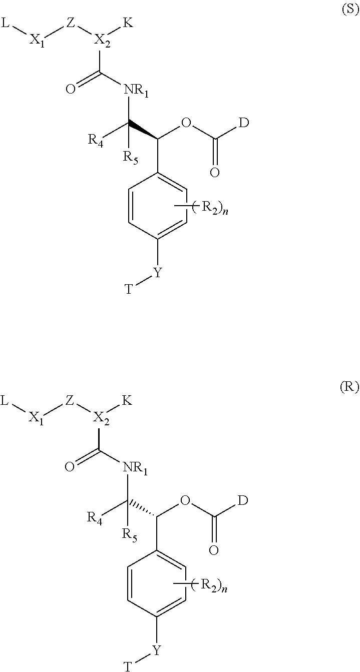

According to an embodiment, the compound of formula (I) is (S).

In a specific embodiment, the compound of formula (I) is a compound of formula (VI):

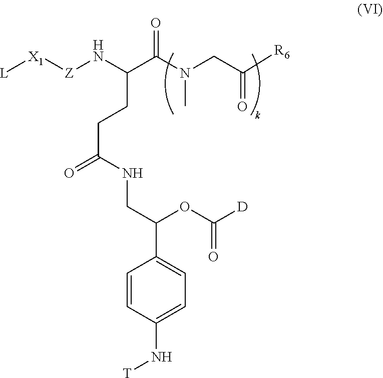

wherein k is an integer between 2 and 50, preferably between 4 and 30; and T is a polypeptide cleavable unit, preferably a dipeptide.

In a specific embodiment, the compound of formula (I) is a compound of formula (VII)

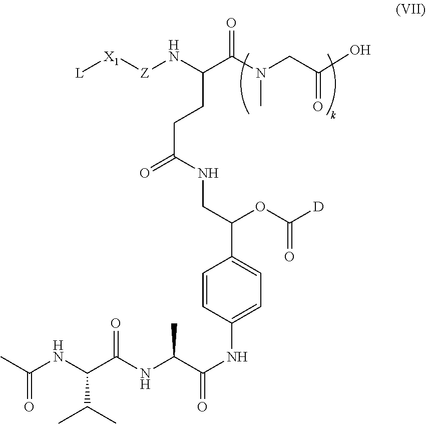

wherein k is an integer between 2 and 50, preferably between 4 and 30.

In a specific embodiment, the compound of formula (I) is a compound of formula (VIII)

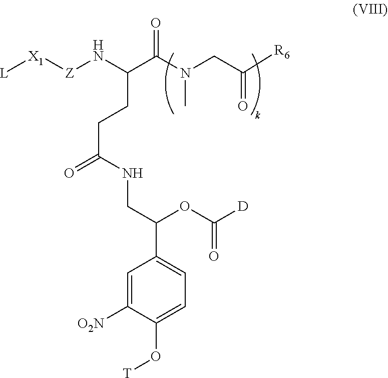

wherein k is an integer between 2 and 50, preferably between 4 and 30; and T is a sugar cleavable unit, preferably a glucuronide or a galactoside.

In a specific embodiment, the compound of formula (I) is a compound of formula (IX)

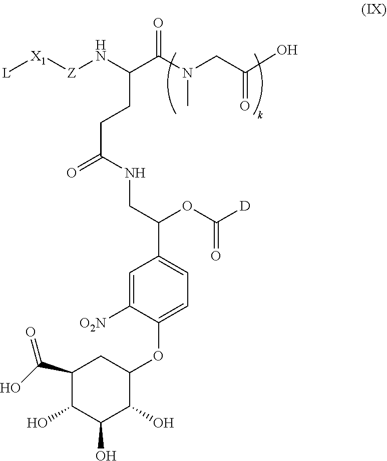

wherein k is an integer between 2 and 50, preferably between 4 and 30.

The compounds of formula (I) according to the invention may be synthesized by any suitable process known in the art, such as the processes disclosed in the examples section.

The embodiments described for the compound of formula (I) also apply for the compounds of formula (II), (III), and (IV).

### Compound of Formula (II)

The present disclosure also relates to compound of formula (II):

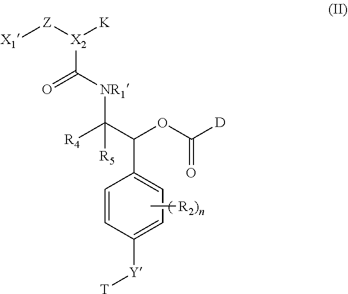

### Wherein

X1′ is a group which can react with a ligand to form a connector unit;
Z is an optional spacer;
X2 is a connector unit;
K is an optional hydrophobicity masking entity, preferably selected from polysarcosine and polyethylene glycol;
R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
D is an active agent, preferably selected from the group consisting of drugs, imaging agents and fluorophores;
each R2 is independently selected from the group consisting of electron-withdrawing groups and C₁-C₄ alkyl;
n is 0, 1 or 2;
R4 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
R5 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
T is a sugar cleavable unit or a polypeptide cleavable unit;
Y′ is O when T is a sugar cleavable unit, or NR3′ when T is a polypeptide cleavable unit;
R3′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R″ being independently selected from H and C₁-C₆ alkyl;
and pharmaceutically acceptable salts thereof.

According to an embodiment, X1′ is a maleimide.

According to an embodiment, R1′ is H.

According to an embodiment, R3′ is H.

The compound of formula (II) can be used to synthesize the compound of formula (I), it is therefore an intermediate in the synthesis of the compound of formula (I).

### Compound of Formula (III)

The present disclosure also relates to a compound of formula (III)

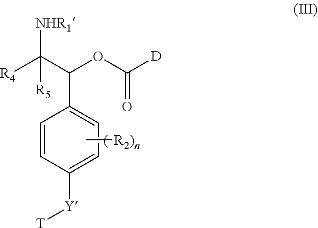

### Wherein

R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
D is an active agent, preferably selected from the group consisting of drugs, imaging agents and fluorophores;
each R2 is independently selected from the group consisting of electron-withdrawing groups;
n is 0, 1 or 2;
R4 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
R5 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
T is a sugar cleavable unit or a polypeptide cleavable unit;
Y′ is O when T is a sugar cleavable unit, or NR3′ when T is a polypeptide cleavable unit;
R3′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR″—, —NR″—C(O)—O—, —O—C(O)NR″— and triazole;
R″ and R″ being independently selected from H and C₁-C₆ alkyl;
and pharmaceutically acceptable salts thereof.

The compound of formula (III) can be used to synthesize the compound of formula (II), it is therefore an intermediate in the synthesis of the compound of formula (I).

### Compound of Formula (IV)

The present disclosure also relates to a compound of formula (IV)

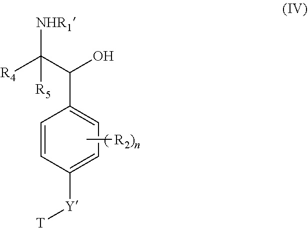

### Wherein

R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatoms or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
each R2 is independently selected from the group consisting of electron-withdrawing groups and C₁-C₄ alkyl;
n is 0, 1 or 2;
R4 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
R5 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
T is a sugar cleavable unit or a polypeptide cleavable unit;
Y′ is O when T is a sugar cleavable unit, or NR3′ when T is a polypeptide cleavable unit;
R3′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R″ being independently selected from H and C₁-C₆ alkyl;
and pharmaceutically acceptable salts thereof.

According to an embodiment, R4 and R5 are H, Y′ is O and T is a sugar cleavable unit.

According to another embodiment, Y′ is NR3′ and T is a polypeptide cleavable unit.

The compound of formula (IV) can be used to synthesize the compound of formula (III), it is therefore an intermediate in the synthesis of the compound of formula (I).

### Pharmaceutical Composition

The disclosure also relates to a pharmaceutical composition comprising a compound of the disclosure and at least one pharmaceutically acceptable carrier. In particular, the present disclosure relates to a pharmaceutical composition comprising a compound of formula (I) and at least one pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. The carrier could be suitable for intravenous, intramuscular, subcutaneous, parenteral, spinal or epidermal administration (e.g., by injection or infusion). In one embodiment, the carrier should be suitable for subcutaneous route or intratumoral injection. Depending on the route of administration, the active compound may be coated in a material to protect the compound from the action of acids and other natural conditions that may inactivate the compound. Formulations may further include one or more excipients, preservatives, solubilizers, buffering agents, etc.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for a topical, oral, intranasal, intraocular, intravenous, intramuscular or subcutaneous administration and the like.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the compound according to the disclosure may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders or lyophilisates for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Compounds of the disclosure can be formulated into a composition in a neutral or salt form. Pharmaceutically acceptable salts include the acid addition salts (formed with free amino groups) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present disclosure, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

The doses used for the administration can be adapted as a function of various parameters, and in particular as a function of the mode of administration used, of the relevant pathology, or alternatively of the desired duration of treatment. It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects. For example, the dose used for the administration can be of about 0.1-1000 mg of the compound of the disclosure for a subject of about 50-70 kg.

### Method of Use

The compounds of the disclosure exhibit valuable pharmaceutical properties as indicated in the in vitro tests and in vivo tests provided in the examples and are therefore indicated for therapy. The disclosure also relates to a compound of the disclosure for use as a drug. In particular, the disclosure relates to a Ligand-Drug Conjugate compound of formula (I), more specifically antibody-drug conjugates compound of formula (1) wherein L is an antibody or antigen-binding portion thereof, for use as a drug.

In particular, the compounds of the disclosure, and more specifically the antibody-drug conjugates of formula (I) of the present disclosure are useful in the prevention or treatment of cancer, inflammatory diseases and/or infectious diseases. In a preferred embodiment, the compound of formula (I) for use in the prevention or the treatment of cancer is an Ligand-Drug conjugate (LDC) of formula (I), wherein L is an antibody or antigen-binding portion thereof, and more preferably wherein D is a cytotoxic compound.

The disclosure also relates to a compound of the disclosure for use in a method for treating cancer. As used herein, the term “cancer” has its general meaning in the art and includes an abnormal state or condition characterized by rapidly proliferating cell growth. The term is meant to include all types of cancerous growths or oncogenic processes, metastatic tissues or malignantly transformed cells, tissues or organs, irrespective of histopathologic type or stage of invasiveness. The term cancer includes malignancies of the various organ systems, such as affecting skin, lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the oesophages.

The disclosure relates to a method for treating cancer said method comprising administering to a subject in need thereof, preferably a human, a therapeutically efficient amount of
(i) a compound of the disclosure, or
(ii) a pharmaceutical composition as described herein.

In preferred embodiments, the disclosure relates to a method for treating or preventing cancer in a subject in need thereof, said method comprising administering a therapeutically effective amount of a Ligand-Drug Conjugate (LDC) of formula (I), wherein L is an antibody or antigen-binding portion thereof, and more preferably wherein D is a cytotoxic compound.

The disclosure also relates to a compound of the disclosure for use in a method for treating inflammatory diseases.

As used herein, the term “inflammatory disease” is used to define any disease caused by, or leading to, inflammation in a subject. The term may include, but is not limited to, (1) inflammatory and/or allergic diseases, (2) autoimmune diseases, (3) graft rejection, and (4) other diseases in which undesired inflammatory responses are to be inhibited.

The disclosure relates to a method for treating an inflammatory disease, said method comprising administering to a subject in need thereof, preferably a human, a therapeutically efficient amount of
(i) a compound of the disclosure, or
(ii) a pharmaceutical composition as described herein.

The disclosure also relates to a compound of the disclosure for use in a method for treating infectious diseases.

As used herein, the term “infectious disease” is used to define any disease caused by the invasion of a subject by infectious agents (or pathogens), their multiplication, and the reaction of the subject's tissues to these infectious agents and the toxins they produce. The term may include, but is not limited to, (1) bacterial infections, (2) viral infections, (3) fungal infections, and (4) parasite infections. The disclosure relates to a method for treating an infectious disease, said method comprising administering to a subject in need thereof, preferably a human, a therapeutically efficient amount of
(i) a compound of the disclosure, or
(ii) a pharmaceutical composition as described herein.
   In preferred embodiments, the disclosure relates to a method for treating or preventing infection disease in a subject in need thereof, said method comprising administering a therapeutically effective amount of a Ligand-Drug Conjugate (LDC) of formula (I), wherein L is an antibody or antigen-binding portion thereof, and more preferably wherein D is an anti-infective agent, for example, an antibiotic or antiviral agent.

As used herein, the term “treating” includes reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

As used herein, the terms “therapeutically efficient amount” of a compound refer to an amount of the compound that will elicit the biological or medical response of a subject, for example, ameliorate the symptoms, alleviate conditions, slow or delay disease progression, or prevent a disease.

The disclosure also relates to the use of a compound of the disclosure, preferably a compound of formula (I), for the manufacture of a medicament for the treatment of cancer, inflammatory diseases and/or infectious diseases, preferably for the treatment of cancer.

The compound of formula (II) can be used as such without the ligand. For example, when X1′ is a maleimide moiety, the compound of formula (II) can react in vivo with a protein, like serum albumin, which then becomes the ligand. Thus, the present disclosure also relates to a compound of formula (II) as described above, for use as a drug.

### EXAMPLES

### Materials and General Methods

All solvents and reagents were obtained from reputable commercial sources (Sigma-Aldrich, Fluorochem, TCI Chemicals, Acros Organics, Alfa Aesar, Enamine, Thermo Fisher, Carbosynth, WuXi AppTec, Iris Biotech) and used without further purification unless stated otherwise. Anhydrous solvents were purchased from Sigma-Aldrich. Fmoc-aminoacids, 2-chlorotrityl, Wang and Rink amide polystyrene 1% DVB 100-200 mesh resins (pre-loaded with first Fmoc-sarcosine aminoacid) were purchased from Christof Senn Laboratories and Sigma-Aldrich. Monomethyl auristatin E (MMAE), Exatecan Mesylate and 7-ethyl-10-hydroxycamptothecin (SN38) were purchased from DCChemicals, MedChemExpress or Abzena. Human albumin (cat #A3782) was purchased from Sigma-Aldrich. Trastuzumab (Herceptin® IV) was purchased from Roche. Non-commercially available monoclonal antibodies were custom-produced by transient transfection on CHO cell line and protein-A/SEC purified by GTP Technologies (Toulouse, France).

On-resin synthesis was performed in empty SPE plastic tubes equipped with a 20 μm polyethylene frit (Sigma-Aldrich). A Titramax 101 platform shaker (Heidolph) was used for agitation. Unless stated otherwise, all chemical reactions were carried out at room temperature under an inert argon atmosphere.

Liquid nuclear magnetic resonance spectra were recorded on a Bruker Fourier 300HD or Bruker AVANCE III HD400 spectrometer, using residual solvent peak for calibration. Mass spectroscopy analysis has been performed by the Centre Commun de Spectrométrie de Masse (CCSM) of the UMR5246 CNRS institute of the University Claude Bernard Lyon 1.

Normal phase flash chromatography was performed on Teledyne Isco CombiFlash® Rf200 devices using Macherey-Nagel Chromabond® flash cartridges (40-63 μm). Reverse phase chromatography was performed using Biotage® Sfär C18 Duo 100 Å 30 μm cartridges or Interchim PuriFlash RP-AQ (30 μm) cartridges on Teledyne Isco Combiflash® Rf200 devices; or using an Agilent 1100 preparative binary HPLC system.

Chemical reactions and compound characterization were respectively monitored and analyzed by thin-layer chromatography using pre-coated 40-63 μm silica gel (Macherey-Nagel), HPLC-UV (Agilent 1100 systems) or UHPLC-UV/MS (Thermo UltiMate 3000 UHPLC system equipped with a Bruker Impact II™ Q-TOF mass spectrometer or Agilent 1260 HPLC system equipped with a Bruker MicrOTOF-QII mass spectrometer).

HPLC Method 1: Agilent 1100 HPLC system equipped with DAD detection. Mobile phase A was water+0.1% TFA and mobile phase B was acetonitrile. Column was an Agilent Zorbax SB-Aq 4.6×150 mm 5 μm (room temperature). Linear gradient was 0% B to 50% B in 30 min, followed by a 5 min hold at 50% B. Flow rate was 1.0 mL/min.

HPLC Method 2: Agilent 1100 HPLC system equipped with DAD detection. Mobile phase A was water+0.1% TFA and mobile phase B was acetonitrile. Column was an Agilent Poroshell 120 EC-C18 3.0×50 mm 2.7 μm (room temperature). Linear gradient was 5% B to 80% B in 9 min, followed by a 1 min hold at 80% B. Flow rate was 0.8 mL/min.

HPLC Method 3: Agilent 1100 HPLC system equipped with DAD detection. Mobile phase A was water+0.1% TFA and mobile phase B was acetonitrile. Column was an Agilent Poroshell 120 EC-C18 3.0×50 mm 2.7 μm (room temperature). Linear gradient was 5% B to 80% B in 20 min, followed by a 2 min hold at 80% B. Flow rate was 0.8 mL/min.

HPLC Method 4: Thermo UltiMate 3000 UHPLC system+Bruker Impact II™ Q-ToF mass spectrometer. Mobile phase A was water+0.1% formic acid and mobile phase B was acetonitrile+0.1% formic acid. Column was an Agilent PLRP-S 1000 Å 2.1×150 mm 8 μm (80° C.). Linear gradient was 10% B to 50% B in 25 min. Flow rate was 0.4 mL/min. UV detection was monitored at 280 nm. The Q-TOF mass spectrometer was used in the m/z range 500-3500 (ESI⁺). Data were deconvoluted using the MaxEnt algorithm included in the Bruker Compass® software.

HPLC Method 5 (preparative method): Teledyne Isco CombiFlash® Rf200 binary MPLC system equipped with DAD detection. Mobile phase A was water+0.1% TFA and mobile phase B was acetonitrile. Reusable cartridges were Biotage® Sfär C18 Duo 100 Å 30 μm (30 g). Linear gradient was 10% B to 50% B in 35 min, followed by a 5 min hold at 50% B. Flow rate was 25 mL/min.

HPLC Method 6 (preparative method): Agilent 1100 preparative binary HPLC system equipped with dual-loop auto-injector, DAD detection and fraction collector. Mobile phase A was water+0.1% TFA and mobile phase B was acetonitrile. Column was a Waters SunFire C18 OBD Prep Column, 100 Å, 5 μm, 19 mm×250 mm (room temperature). Linear gradient was 10% B to 60% B in 40 min, followed by a 5 min hold at 60% B. Flow rate was 25 mL/min.

### 1) Preparation of Chemical Compounds of the Invention

### 1.1) Monodisperse Polysarcosines Intermediates

### 1.1.1) General Methods

On-resin synthesis of monodisperse polysarcosines were realized using sub-monomer synthesis iterative procedures (already described in patent WO2019081455) for Rink amide and 2-chlorotrityl resin or following classic Fmoc/SPPS methodologies with the commercial Fmoc-Sar-Sar-OH dipeptoid building block (CAS #2313534-20-0) for Wang resin. Please note that peptoidic sub-monomer synthesis on Wang resin were unsuccessful. In all cases, on-resin dimeric stage (n=2) was avoided due to diketopiperazine formation. All synthesis yields are reported based upon initial Fmoc-sarcosine loading indicated by the manufacturer. Unless stated otherwise, all reactions were carried out at room temperature. Rink amide, 2-chlorotrityl or Wang polystyrene 1% DVB 100-200 mesh resins preloaded with a first Fmoc-sarcosine residue (Christof Senn Laboratories) were used (typical initial loading of 0.6-1 mmol/g).

### 1.1.2) Elongation of Polysarcosines

Fmoc-sarcosine preloaded Rink amide, 2-chlorotrityl or Wang resin was treated with 20% piperidine in DMF (1 mL per 100 mg of resin) for 2 times 15 min at room temperature. The resin was then washed with DMF (4 times) and DCM (4 times). To the resin was added a solution of Fmoc-Sar-Sar-OH (3 eq), HATU (2.9 eq) and DIPEA (6 eq) in DMF (1 mL per 100 mg of resin). The reaction vessel was agitated for 2 hours and the resin was washed with DMF (4 times) and DCM (4 times). The resin was treated with 20% piperidine in DMF (1 mL per 100 mg of resin) for 2 times 15 min at room temperature. The resin was then washed with DMF (4 times) and DCM (4 times).

For synthesis on Rink amide or 2-chlorotrityl resin, classic sub-monomer synthesis procedures were then used, as described in WO2019081455. Elongation of the n=3 polysarcosine oligomer was performed until the desired length was obtained, by alternating bromoacetylation and amine displacement steps. The bromoacetylation step was performed by adding 10 eq of bromoacetic acid and 13 eq of diisopropylcarbodiimide in DMF (2 mL per 100 mg of resin). The mixture was agitated for 30 min, drained and washed with DMF (4 times). For the amine displacement step, a 40% (wt) methylamine in water solution was added (1.5 mL per 100 mg of resin) and the vessel was shaken for 30 min, drained and washed with DMF (4 times) and DCM (4 times).

For synthesis on Wang resin, classic Fmoc/SPPS procedures were used. Elongation of the n=3 polysarcosine oligomer was performed by iterative coupling of Fmoc-Sar-Sar-OH dipeptoid building block (CAS #2313534-20-0). To the resin was added a solution of Fmoc-Sar-Sar-OH (3 eq), HATU (2.9 eq) and DIPEA (6 eq) in DMF (1 mL per 100 mg of resin). The reaction vessel was agitated for 90 min and the resin was extensively washed with DMF (4 times) and DCM (4 times). Resin was then treated with 20% piperidine in DMF (1 mL per 100 mg of resin) for 2 times 15 min at room temperature. The resin was washed with DMF (4 times) and DCM (4 times). This coupling/Fmoc-deprotection cycle was repeated until desired polysarcosine length is obtained. If necessary, the last coupling is made with commercial Fmoc-Sar-OH aminoacid instead of Fmoc-Sar-Sar-OH dipeptoid unit in order to obtain a final polysarcosine of even length.

### 1.1.3) Final On-Resin Side-Functionalization of Polysarcosines, Optional Capping, Resin Cleavage and Purification

When the desired on-resin polysarcosine monodisperse oligomer length is reached, orthogonal chemical functionalization is performed. It is optionally followed by a final capping with a Fmoc-aminoacid (for example Fmoc-Gly-OH, Fmoc-β-Ala-OH, Fmoc-Amino-3,6 dioxaoctanoic acid, Fmoc-9-Amino-4,7-Dioxanonanoic acid). The Fmoc protecting group capping the N-terminus of the final compound can be removed before or after resin cleavage, depending on the orthogonal functionalization chemistry that is used (see after).

### 1.1.3.1) 2-Azidoethan-1-Amine Side-Functionalized Polysarcosines

To the Rink or 2-chlorotrityl resin is added 10 eq of bromoacetic acid and 13 eq of diisopropylcarbodiimide in DMF (2 mL per 100 mg of resin). The mixture was agitated for 30 min, drained and washed with DMF (4 times). A 3 molar solution of 2-azidoethan-1-amine in DMF was added (1 mL per 100 mg of resin) and the vessel was shaken for 45 min, drained and washed with DMF (4 times) and DCM (4 times). It was followed by a 1-hour Fmoc-Gly-OH coupling (5 eq Fmoc-Gly-OH, 4.9 eq HATU, 10 eq DIPEA in DMF (1 mL per 100 mg of resin) and Fmoc-deprotection with 20% piperidine in DMF (1 mL per 100 mg of resin) for 2 times 15 min at room temperature. The resin was washed with DMF (4 times) and DCM (4 times).

Final polysarcosine compounds were cleaved from the resin (100% TFA 2 times 30 min for Rink and Wang resins, 20% TFA in DCM 2 times 15 min for 2-chlorotrityl resin). Resin was filtered, and volatiles were removed under reduced pressure to afford a crude that was purified on Interchim® RP-AQ (30 μm) cartridges. Mobile phase A was water+0.1% TFA and mobile phase B was acetonitrile.

### 1.1.3.2) β-Alanine Side-Functionalized Polysarcosines

To the Rink or 2-chlorotrityl resin is added 10 eq of bromoacetic acid and 13 eq of diisopropylcarbodiimide in DMF (2 mL per 100 mg of resin). The mixture was agitated for 30 min, drained and washed with DMF (4 times). A 3 molar solution of allyl 3-aminopropanoate (synthesized as described in Schröer et al., J. Org. Chem. 1997, 62, 10, 3220-3229 and freebased 20 min at 50° C. with 3 eq Na₂CO₃ in THF) in DMF was added (1 mL per 100 mg of resin) and the vessel was shaken for 45 min, drained and washed with DMF (4 times) and DCM (4 times). It was followed by a 1-hour Fmoc-Gly-OH coupling (5 eq Fmoc-Gly-OH, 4.9 eq HATU, 10 eq DIPEA in DMF (1 mL per 100 mg of resin). The resin was washed with DMF (4 times), DCM (4 times). Alloc-protecting group was removed by a 2 times 30 min treatment with a DCM solution containing 0.25 eq of Pd(PPh₃)₄ and 20 eq of phenylsilane (gently agitated under a stream of argon). The resin was then washed with DMF (5 times) and DCM (5 times). Optionally an N-hydroxysuccinimide (NHS) ester was introduced to the carboxylic acid side chain of the final polysarcosine compound, by a 90 min treatment with a DMF solution containing 50 eq of DIC and 60 eq of N-hydroxysuccinimide (1.5 mL per 100 mg of resin). The resin was then washed with DMF (4 times) and DCM (4 times).

Final polysarcosine compounds were cleaved from the resin (100% TFA 2 times 30 min for Rink and Wang resins, 20% TFA in DCM 2 times 15 min for 2-chlorotrityl resin). Resin was filtered, and volatiles were removed under reduced pressure to afford a crude that was purified on Interchim® RP-AQ (30 μm) cartridges. Mobile phase A was water+0.1% TFA and mobile phase B was acetonitrile.

### 1.1.3.3) Glutamic Acid Side-Functionalized Polysarcosines

To the Rink, Wang or 2-chlorotrityl resin was added a solution of Fmoc-Glu(OAII)—OH (3 eq), HATU (2.9 eq) and DIPEA (6 eq) in DMF (1 mL per 100 mg of resin). The reaction vessel was agitated for 90 min and the resin was extensively washed with DMF (4 times) and DCM (4 times). Resin was then treated with 20% piperidine in DMF (1 mL per 100 mg of resin) for 2 times 15 min at room temperature. The resin was washed with DMF (4 times) and DCM (4 times). It was followed by a 1-hour coupling with Fmoc-Amino-3,6 dioxaoctanoic acid (3 eq), HATU (2.9 eq), DIPEA (6 eq) in DMF (1 mL per 100 mg of resin). The resin was washed with DMF (4 times), DCM (4 times). Alloc-protecting group was removed by a 2 times 30 min treatment with a DCM solution containing 0.25 eq of Pd(PPh₃)₄ and 20 eq of phenylsilane (gently agitated under a stream of argon). The resin was then washed with DMF (5 times) and DCM (5 times). Optionally an N-hydroxysuccinimide (NHS) ester was introduced to the carboxylic acid side chain of the final polysarcosine compound, by a 90 min treatment with a DMF solution containing 50 eq of DIC and 60 eq of N-hydroxysuccinimide (1.5 mL per 100 mg of resin). The resin was then washed with DMF (4 times) and DCM (4 times).

Final polysarcosine compounds were cleaved from the resin (100% TFA 2 times 30 min for Rink and Wang resins, 20% TFA in DCM 2 times 15 min for 2-chlorotrityl resin). Resin was filtered, and volatiles were removed under reduced pressure to afford a crude that was purified on Interchim® RP-AQ (30 μm) cartridges. Mobile phase A was water+0.1% TFA and mobile phase B was acetonitrile.

### 1.1.3.4) γ-Azidohomoalanine-Functionalized Polysarcosines

To the Rink, Wang or 2-chlorotrityl resin was added a solution of Fmoc-γ-azidohomoalanine (3 eq), HATU (2.9 eq) and DIPEA (6 eq) in DMF (1 mL per 100 mg of resin). The reaction vessel was agitated for 90 min and the resin was extensively washed with DMF (4 times) and DCM (4 times). Resin was then treated with 20% piperidine in DMF (1 mL per 100 mg of resin) for 2 times 15 min at room temperature. The resin was washed with DMF (4 times) and DCM (4 times). It was followed by a 1-hour coupling with Fmoc-Amino-3,6 dioxaoctanoic acid (3 eq), HATU (2.9 eq), DIPEA (6 eq) in DMF (1 mL per 100 mg of resin). The resin was washed with DMF (4 times), DCM (4 times).

Final polysarcosine compounds were cleaved from the resin (100% TFA 2 times 30 min for Rink and Wang resins, 20% TFA in DCM 2 times 15 min for 2-chlorotrityl resin). Resin was filtered, and volatiles were removed under reduced pressure to afford a crude that was purified on Interchim® RP-AQ (30 μm) cartridges. Mobile phase A was water+0.1% TFA and mobile phase B was acetonitrile.

### 1.1.4) Final Polysarcosine Intermediates

The following table 1 lists the resulting compounds.

TABLE 1

| | | | | | HPLC retention |
|---|---|---|---|---|---|
| Compound name | Structure | Resin | Yield | MS (ESI⁺) | time |
| Compound A1 NHFmoc-Gly- (βAlanine-NHS)- PSAR10-CONH₂ (white solid) | 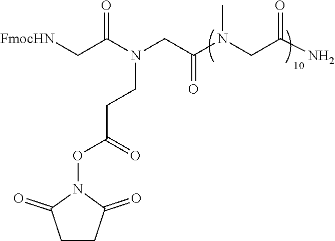 | Rink amide | 56% | Calc [M + H]⁺ = 1233.5 Obs [M + H]⁺ = 1233.5 | 9.1 min (HPLC method 3) |
| Compound A2 NH₂-Gly-(N₃)- PSAR10- CONH₂ (transparent oil) | 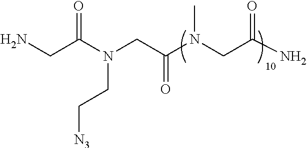 | Rink amide | 48% | Calc [M + H]⁺ = 911.5 Obs [M + H]⁺ = 911.5 | 12.5 min (HPLC method 1) |
| Compound A3 NH₂-Gly-(N₃)- PSAR10- COOH (transparent oil) | 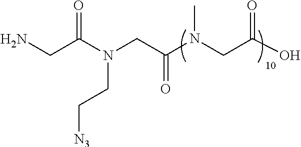 | 2-chlorotrityl | 14% | Calc [M + H]⁺ = 912.5 Obs [M + H]⁺ = 912.5 | 14.5 min (HPLC method 1) |
| Compound A4 NHFmoc-Gly- (βAlanine- NHS)-PSAR12- CONH₂ (white solid) | 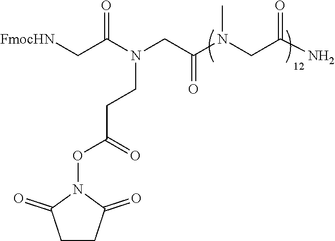 | Rink amide | 50% | Calc [M + Na]⁺ = 1375.6 Obs [M + Na]⁺ = 1375.6 | 5.5 min (HPLC method 2) |
| Compound A5 NH₂-Gly-(N₃)- PSAR12- CONH₂ (transparent oil) | 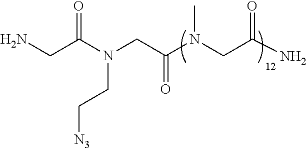 | Rink amide | 37% | Calc [M + H]⁺ = 1053.5 Obs [M + H]⁺ = 1053.5 | 11.6 min (HPLC method 1) |
| Compound A6 NHFmoc-Gly- (Glu-NHS)- PSAR10-CONH₂ (white solid) | 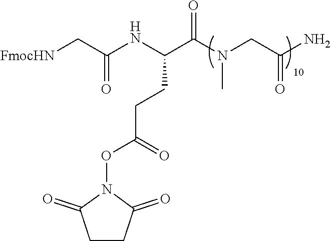 | Rink amide | 56% | Calc [M + 2Na]²⁺ = 639.3 Obs [M + 2Na]²⁺ = 639.3 | 5.4 min (HPLC method 2) |
| Compound A7 NHFmoc- PEG2-(Glu- NHS)-PSAR10- COOH (white solid) | 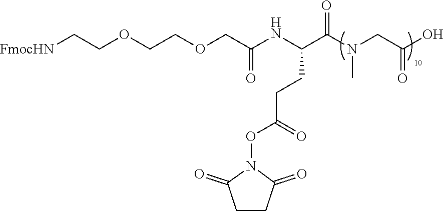 | Wang | 60% | Calc [M + 2H]²⁺ = 661.8 Obs [M + 2H]²⁺ = 661.8 | 5.7 min (HPLC method 2) |
| Compound A8 NH₂-PEG2-(N₃)- PSAR10- COOH (transparent oil) | 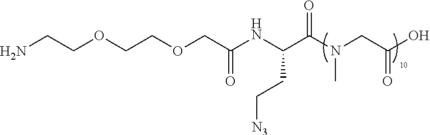 | Wang | 65% | Calc [M + H]⁺ = 1000.5 Obs [M + H]⁺ = 1000.5 | 14.0 min (HPLC method 1) |

### 1.2) Intermediate Compounds Synthesis

### 1.2.1) Synthesis of Compound B1 and Compound B2

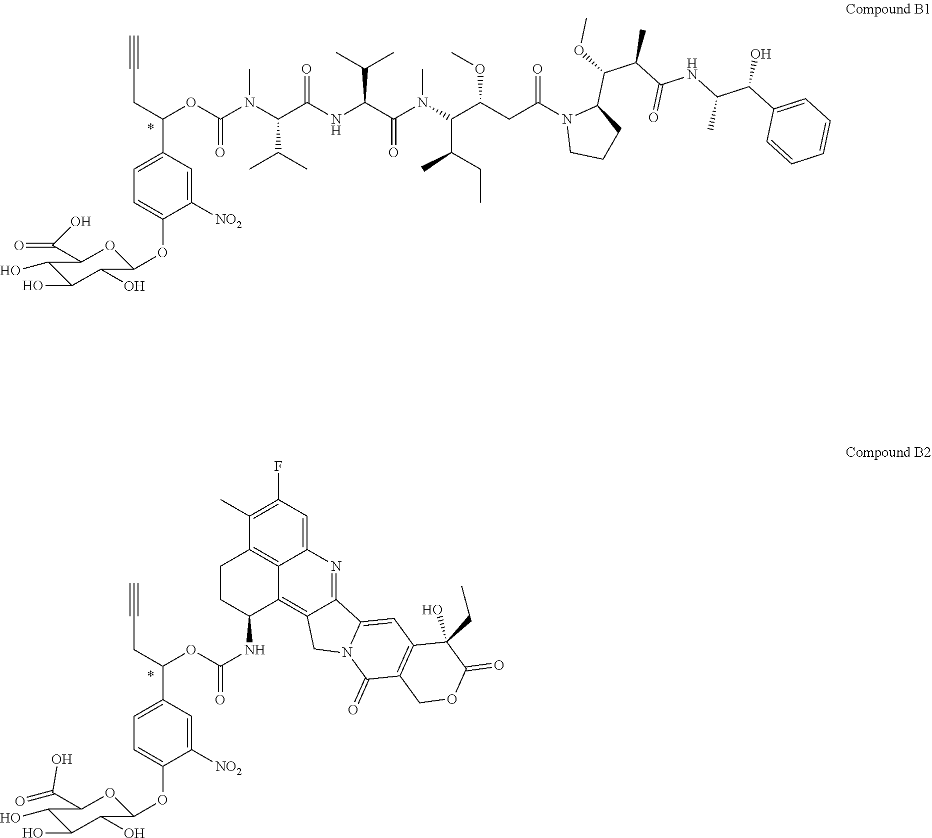

These compounds were synthesized following procedures described in patent WO2019081455. These compounds are equimolar diastereoisomeric mixtures (stereogenic center indicated by an asterisk).

### 1.2.2) Synthesis of Compound B3 and Compound B4

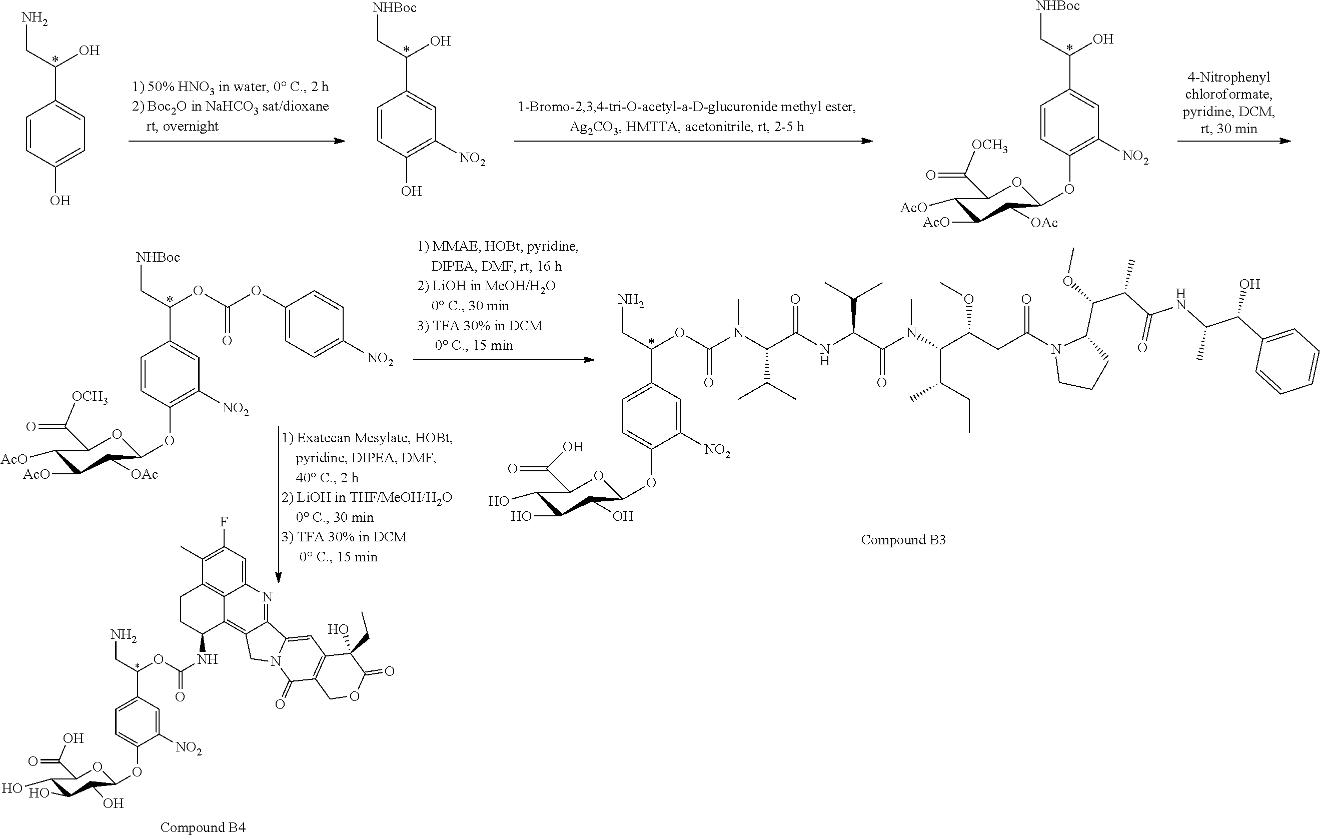

### 1.2.2.1) Synthesis of tert-butyl (2-hydroxy-2-(4-hydroxy-3-nitrophenyl)ethyl)carbamate

(±)-octopamine hydrochloride (1690 mg/11 mmol) was weighted in a round-bottom flask and suspended in 4 mL of distilled water. The flask was chilled at 0° C. and 4 mL of a pre-chilled 65% nitric acid solution was slowly added. The reaction was kept at 0° C. for 20 minutes, showing complete mono-nitration of the starting material as assessed by HPLC. The content of the flask was transferred in a 250 ml pre-chilled Erlenmeyer and slowly neutralized at 0° C. with a saturated NaHCO₃ solution (approx. 50 mL) until a pH value of 8-9 is reached. 30 mL of dioxane was then added, followed by Boc₂O (7202 mg/13.2 mmol). The reaction was then allowed to reach room temperature and was stirred overnight. The reaction was then diluted with EtOAc and washed 3 times with a saturated citric acid solution and once with saturated NaCl solution. The organic phase was dried over MgSO₄, filtered and evaporated under vacuum to afford a crude that was purified by chromatography on silica gel (petroleum ether/EtOAc, gradient from 70:30 to 20:80) to afford title compound (1320 mg/40%) as a thick yellow-to-brown oil. ¹H NMR (300 MHZ, DMSO-d₆) δ 10.79 (s, 1H), 7.79 (d, J=2.1 Hz, 1H), 7.47 (dd, J=8.6, 2.1 Hz, 1H), 7.08 (d, J=8.6 Hz, 1H), 6.74 (t, J=5.9 Hz, 1H), 4.56 (t, J=6.3 Hz, 1H), 3.07 (td, J=6.1, 1.6 Hz, 2H), 1.31 (s, 9H). MS m/z (ESI⁺): Calc [M+H]⁺=299.1; Exp [M+H]⁺=299.1. HPLC Method 2 retention time=5.5 min.

### 1.2.2.2) Synthesis of (2S,3R,4S,5S,6S)-2-(4-(2-((tert-butoxycarbonyl)amino)-1-hydroxyethyl)-2-nitrophenoxy)-6-(methoxycarbonyl)tetrahydro-2H-pyran-3,4,5-triyl triacetate

In a round-bottom flash, Ag₂CO₃ (1500 mg/5.4 mmol) and 1,1,4,7,10,10-hexamethyltriethylenetetramine (251 mg/1.1 mmol) were taken up in 4 mL of anhydrous acetonitrile and stirred during 2 hours at room temperature. Previous compound tert-butyl (2-hydroxy-2-(4-hydroxy-3-nitrophenyl)ethyl)carbamate (292 mg/0.98 mmol) and 1-bromo-2,3,4-tri-O-acetyl-α-D-glucuronide methyl ester (583 mg/1.46 mmol) were added at 0° C. and the solution mixture was stirred for 4 h at room temperature. The reaction was then filtered on celite, diluted with EtOAc, and washed 3 times with a saturated citric acid solution and once with a saturated NaCl solution. The organic phase was dried over MgSO₄, filtered and evaporated under vacuum to afford a crude that was purified by chromatography on silica gel (petroleum ether/EtOAc, gradient from 70:30 to 30:70) to afford title compound (244 mg/48%) as a yellow foam. ¹H NMR (300 MHZ, DMSO-d₆) δ 7.76 (dd, J=3.3, 2.1 Hz, 1H), 7.60 (t, J=7.6 Hz, 1H), 7.36 (dd, J=8.7, 2.6 Hz, 1H), 6.77 (s, 1H), 5.71 (d, J=7.8 Hz, 1H), 5.61 (s, 1H), 5.46 (td, J=9.5, 1.1 Hz, 1H), 5.21-5.02 (m, 3H), 4.75 (dd, J=9.9, 1.4 Hz, 1H), 4.62 (s, 1H), 3.65 (s, 3H), 3.17 (s, 2H), 3.10 (t, J=6.1 Hz, 2H), 2.81-2.59 (m, 6H), 2.05-1.96 (m, 9H), 1.30 (d, J=1.7 Hz, 9H). MS m/z (ESI⁺): Calc [M+Na]⁺=637.2; Exp [M+Na]⁺=637.2. HPLC Method 2 retention time=6.75 min.

### 1.2.2.3) Synthesis of (2S,3R,4S,5S,6S)-2-(4-(2-((tert-butoxycarbonyl)amino)-1-(((4-nitrophenoxy)carbonyl)oxy)ethyl)-2-nitrophenoxy)-6-(methoxycarbonyl)tetrahydro-2H-pyran-3,4,5-triyl triacetate

Previous compound (2S,3R,4S,5S,6S)-2-(4-(2-((tert-butoxycarbonyl)amino)-1-hydroxyethyl)-2-nitrophenoxy)-6-(methoxycarbonyl)tetrahydro-2H-pyran-3,4,5-triyl triacetate (334 mg/0.54 mmol) and 4-nitrophenyl chloroformate (219 mg/1.09 mmol) were dissolved in 6 mL of dry DCM at 0° C. Anhydrous pyridine (112 mg/1.41 mmol) was added and the mixture was stirred 30 min at room temperature. The reaction was filtered over a 0.45 μm PTFE filter and purified by chromatography on silica gel (petroleum ether/EtOAc, gradient from 85:15 to 30:70) to afford title compound (380 mg/90%) as a yellow foam. ¹H NMR (300 MHZ, DMSO-d₆) δ 8.39-8.26 (m, 2H), 7.93 (d, J=2.2 Hz, 1H), 7.75 (d, J=8.8 Hz, 1H), 7.55 (dd, J=9.2, 1.2 Hz, 2H), 7.46 (d, J=8.8 Hz, 1H), 7.20 (d, J=4.8 Hz, 1H), 5.77 (dd, J=7.7, 3.7 Hz, 1H), 5.47 (t, J=9.5 Hz, 1H), 5.11 (q, J=9.6 Hz, 2H), 4.77 (d, J=9.9 Hz, 1H), 3.73-3.59 (m, 3H), 3.59-3.37 (m, 2H), 2.05-1.96 (m, 9H), 1.48-1.35 (m, 1H), 1.32 (s, 9H). MS m/z (ESI⁺): Calc [M+Na]⁺=802.15; Exp [M+Na]⁺=802.15. HPLC Method 2 retention time=8.5 min.

### 1.2.2.4) Synthesis of Compound B3

101 mg (0.13 mmol) of previous compound (2S,3R,4S,5S,6S)-2-(4-(2-((tert-butoxycarbonyl)amino)-1-(((4-nitrophenoxy)carbonyl)oxy)ethyl)-2-nitrophenoxy)-6-(methoxycarbonyl)tetrahydro-2H-pyran-3,4,5-triyl triacetate, 98 mg (0.14 mmol) of MMAE and 18 mg (0.13 mmol) of HOBt were dissolved in 1 mL of a 85:15 (v/v) mixture of anhydrous DMF/pyridine. 16.7 mg (0.13 mmol) of DIPEA was added. The reaction was stirred 16 hours at room temperature and volatiles were evaporated under reduced pressure. The crude residue was purified by chromatography on silica gel (DCM/MeOH gradient from 99:1 to 95:5) to afford 147 mg (84%) of intermediate compound (white solid) that was directly engaged into the deprotection step. ESI⁺[M+H]⁺=1358.7. HPLC Method 2 retention time=8.8 min.

144 mg (0.106 mmol) of this intermediate compound was dissolved in 3 ml of MeOH at 0° C. LiOH monohydrate (44.5 mg/1.06 mmol) was dissolved in water (0.4 mL) and was added to the reaction vessel. After stirring at 0° C. for 30 min (reaction followed by HPLC), the mixture was neutralized with acetic acid (83 mg/1.4 mmol) and concentrated under reduced pressure. The obtained crude was re-dissolved at 0° C. with a TFA/DCM (30:70 v/v) solution and stirred 15 minutes at room temperature. Volatiles were evaporated under reduced pressure, the crude residue was taken up in a water/ACN (1:1 v/v) solution and purified using HPLC preparative method 5 to afford 85 mg (72%) of compound B3 as a white solid. HRMS m/z (ESI⁺): Calc [M+H]⁺=1118.5867; Exp [M+H]⁺=1118.5842; Error=2.2 ppm. HPLC Method 3 retention time=9.36 min.

### 1.2.2.5) Synthesis of Compound B4

Compound B4 was synthesized following the same procedures that were used for synthesis of compound B3 with slight adjustments. (2S,3R,4S,5S,6S)-2-(4-(2-((tert-butoxycarbonyl)amino)-1-(((4-nitrophenoxy)carbonyl)oxy)ethyl)-2-nitrophenoxy)-6-(methoxycarbonyl)tetrahydro-2H-pyran-3,4,5-triyl triacetate coupling reaction with Exatecan Mesylate was conducted at 40° C. for 2 hours instead of overnight at room temperature.

125 mg (87%) of intermediate compound (2S,3R,4S,5S,6S)-2-(4-(2-((tert-butoxycarbonyl)amino)-1-((((1R,9R)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl) carbamoyl)oxy)ethyl)-2-nitrophenoxy)-6-(methoxycarbonyl)tetrahydro-2H-pyran-3,4,5-triyl triacetate was obtained at a yellow/greenish solid. ESI⁺[M+Na]⁺=1098.3. HPLC Method 3 retention time=14.7 and 14.9 min (diastereoisomeric mixture).

Deprotection of the glucuronide moiety was conducted with MeOH/THF (75:25 v/v) instead of pure MeOH. Boc-deprotection was conducted as described for compound B3.

Purification using HPLC preparative method 5 afforded 65 mg (67%) of compound B4 as a yellow solid. ESI⁺[M+H]⁺=836.2. HPLC Method 3 retention time=7.3 and 7.8 min (diastereoisomeric mixture).

### 1.2.2.6) Synthesis of Stereopure Compounds B4-S and B4-R

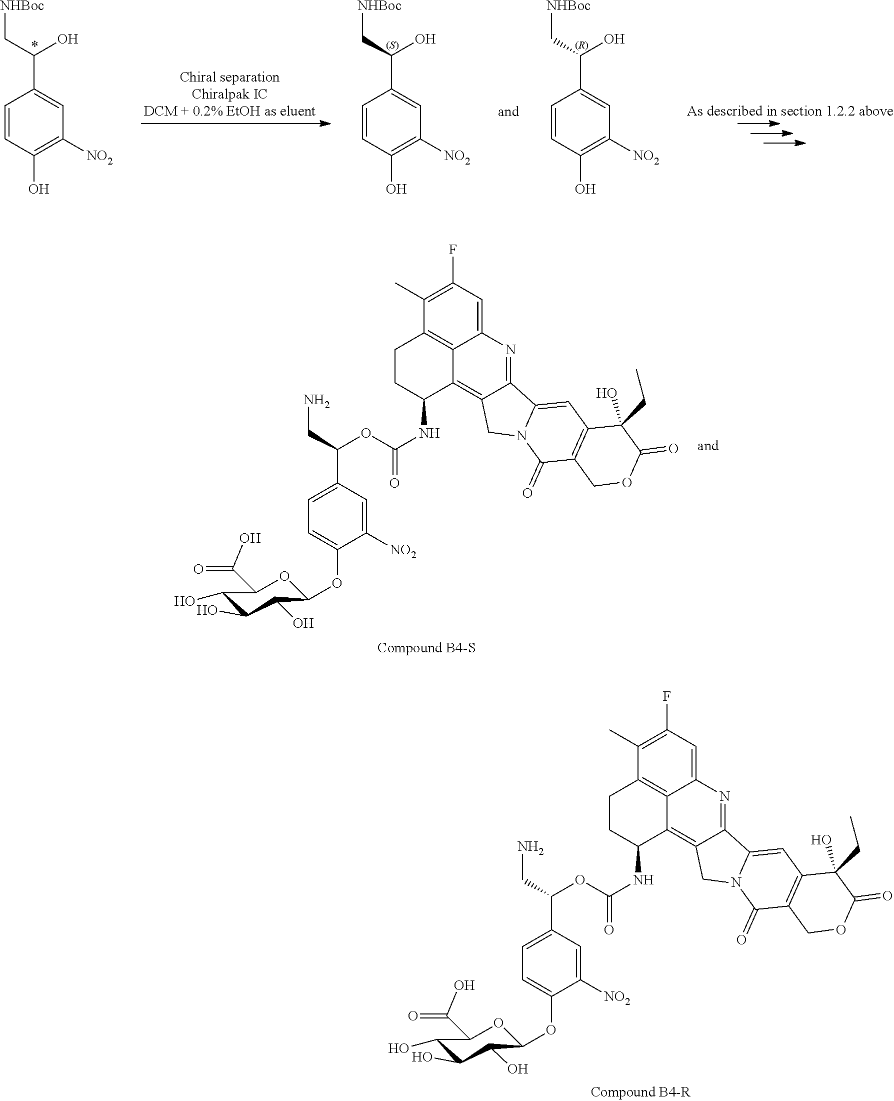

### 1.2.2.6.1) Chiral Separation of Racemic Mixture of tert-butyl (2-hydroxy-2-(4-hydroxy-3-nitrophenyl)ethyl)carbamate

Chiral separation of racemic tert-butyl (2-hydroxy-2-(4-hydroxy-3-nitrophenyl)ethyl)carbamate (synthesized as previously described) was performed using Chiralflash® IC MPLC column 30×100 mm, 20 μm (Daicel cat #83M73) on a Teledyne Isco CombiFlash® Rf200 system. Mobile phase was DCM+0.2% (v/v) EtOH (isocratic gradient). Flow rate was 12 mL/min. Sample solvent was DCM+0.2% (v/v) EtOH. Mass recovery of the two enantiomers after separation was above 80%.

tert-butyl (S)-(2-hydroxy-2-(4-hydroxy-3-nitrophenyl)ethyl)carbamate retention time was 15 min, whereas tert-butyl (R)-(2-hydroxy-2-(4-hydroxy-3-nitrophenyl)ethyl)carbamate retention time was 25 min.

To determine absolute configuration, phenolic position of both enantiomers was esterified with 1.2 molar equivalents of 4-nitrobenzoyl chloride and 2 molar equivalents of triethylamine in anhydrous THF. Compounds were purified by chromatography on silica gel (petroleum ether/EtOAc, gradient from 90:10 to 10:90) to afford 4-(2-((tert-butoxycarbonyl)amino)-1-hydroxyethyl)-2-nitrophenyl 4-nitrobenzoate. ¹H NMR (300 MHz, DMSO-d₆) δ 8.51 (d, J=9.1 Hz, 2H), 8.44 (d, J=9.1 Hz, 2H), 8.19 (d, J=1.9 Hz, 1H), 7.88 (d, J=10.2 Hz, 1H), 7.72 (d, J=8.4 Hz, 1H), 6.93 (t, J=5.9 Hz, 1H), 5.84 (d, J=4.7 Hz, 1H), 4.86-4.76 (m, 1H), 3.24 (t, J=6.1 Hz, 2H), 1.38 (s, 9H). ESI⁺[M+Na]⁺=470.1. Absolute configuration of enantiomers (previously dissolved in a 1:1 mixture of heptane/dichloromethane and allowed to slowly evaporate for 3 weeks to induce the formation of crystals) was confirmed by x-ray crystallography. A block-shaped crystal was mounted on a nylon loop in perfluoroether oil. Data were collected using a Xcalibur, Atlas, Gemini ultra-diffractometer equipped with an Oxford Cryosystems low-temperature device operating at T=150.00 (5) K. Data were measured using ω scans using Cu K_{α} radiation. The structure was solved with the ShelXT solution program using dual methods and by using Olex2 (O. V. Dolomanov et al., Olex2: A complete structure solution, refinement and analysis program, J. Appl. Cryst., 2009, 42, 339-341) as the graphical interface. The model was refined with ShelXL 2018/3 (Sheldrick, G. M., Crystal structure refinement with ShelXL, Acta Cryst., 2015, C71, 3-8) using full matrix least squares minimisation on F².

### 1.2.2.6.2) Synthesis of Stereopure B4-S and B4-R Compounds

Stereopure compounds B4-S and B4-R were synthesized as described in previous section 1.2.2, without any appreciable changes in reaction conditions, reactivity or overall yields.

Final purification using HPLC preparative method 5 afforded 33 mg of compound B4-S as a yellow solid. ESI⁺ [M+H]⁺=836.2. HPLC Method 3 retention time=7.3 min.

Final purification using HPLC preparative method 5 afforded 21 mg of compound B4-R as a yellow solid. ESI⁺ [M+H]⁺=836.2. HPLC Method 3 retention time=7.8 min.

### 1.2.3) Synthesis of Compound B5 and Compound B6

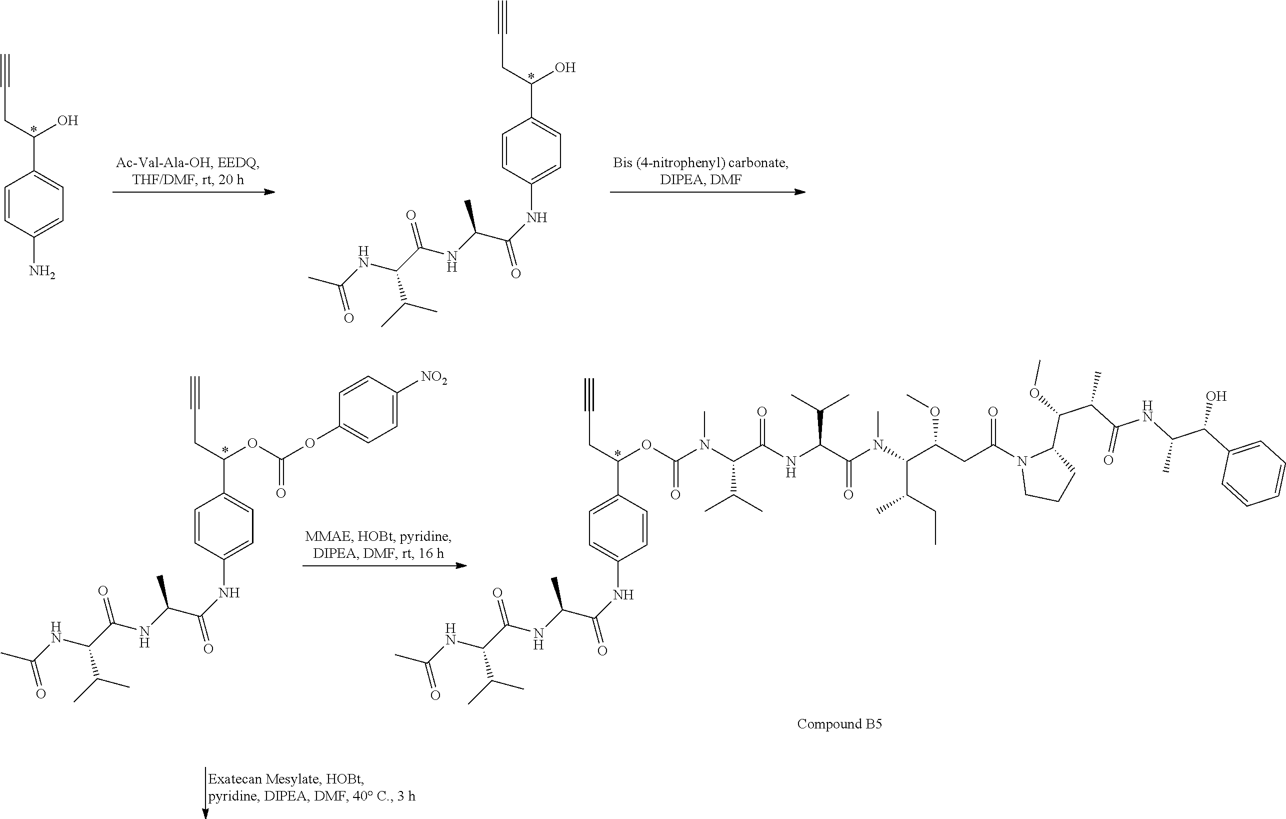 [SMILES: C#CCC(O)c1ccc(N)cc1 ; C#CCC(O)c1ccc(NC(=O)[C@H](C)NC(=O)[C@@H](NC(C)=O)C(C)C)cc1 ; C#CCC(OC(=O)Oc1ccc([N+](=O)[O-])cc1)c1ccc(NC(=O)[C@H](C)NC(=O)[C@@H](NC(C)=O)C(C)C)cc1 ; C#CCC(OC(=O)N(C)[C@H](C(=O)N[C@H](C(=O)N(C)[C@@H]([C@@H](C)CC)[C@@H](CC(=O)N1CCC[C@H]1[C@H](OC)[C@H](C)C(=O)N[C@@H](C)[C@H](O)c1ccccc1)OC)C(C)C)C(C)C)c1ccc(NC(=O)[C@H](C)NC(=O)[C@@H](NC(C)=O)C(C)C)cc1]

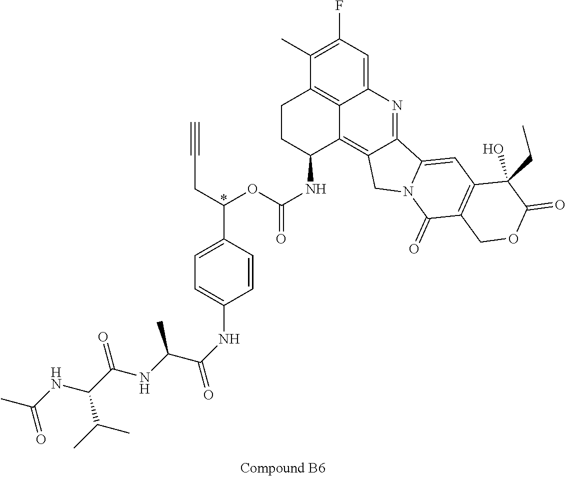 [SMILES: C#CCC(OC(=O)N[C@H]1CCc2c(C)c(F)cc3nc4c(c1c23)Cn1c-4cc2c(c1=O)COC(=O)[C@]2(O)CC)c1ccc(NC(=O)[C@H](C)NC(=O)[C@@H](NC(C)=O)C(C)C)cc1]

### 1.2.3.1) Synthesis of Ac-Val-Ala-OH (acetyl-L-valyl-L-alanine)

To a solution of L-alanine benzyl ester hydrochloride (542 mg/2.5 mmol) in 30 mL of DCM were sequentially added triethylamine (254 mg/2.5 mmol), distilled water (30 mL), N-α-Acetyl-L-valine (400 mg/2.5 mmol) and HOBt (339 mg/2.5 mmol). The mixture was then cooled to 0° C. and EDC-HCl (530 mg/2.75 mmol) was added. The resulting mixture was stirred at 0° C. overnight. The reaction was diluted with 20 ml of 2M HCl and the layers were separated. The organic phase was washed 2 times with 2M HCl, 2 times with saturated NaHCO₃ solution and once with saturated NaCl solution. The organic phase was dried over MgSO₄, filtered and evaporated under vacuum to afford 714 mg (89%) of benzyl acetyl-L-valyl-L-alaninate as a white solid intermediate.

This intermediate was solubilized in 10 mL of EtOAc/MeOH 1:1 (v/v) and was transferred into a stainless steel hydrogenation reactor. After a first argon purge, a catalytic amount of 5% wt Pd/C was added. The reactor was then purged twice with H₂ and kept under a H₂ pressure of 10 bar overnight at room temperature. After filtration of the reaction with a 0.45 μm PTFE filter and solvent removal under vacuum, a quantitative amount of pure acetyl-L-valyl-L-alanine was obtained as a white solid. 1H NMR (300 MHZ, DMSO-d₆) δ 12.45 (s, 1H), 8.23 (d, J=6.9 Hz, 1H), 7.85 (d, J=9.0 Hz, 1H), 4.25-4.11 (m, 2H), 1.94 (dt, J=13.6, 6.8 Hz, 1H), 1.85 (s, 3H), 1.26 (d, J=7.3 Hz, 3H), 0.85 (dd, J=12.1, 6.8 Hz, 6H).

### 1.2.3.2) Synthesis of (2S)-2-acetamido-N-((2S)-1-((4-(1-hydroxybut-3-yn-1-yl)phenyl)amino)-1-oxopropan-2-yl)-3-methylbutanamide

In a round bottom flash, 420 mg (2.60 mmol) of 1-(4-aminophenyl)but-3-yn-1-ol (synthesized following procedures described in Sharma A. et al., Chem 2018, 4 (10), 2370-2383) and 600 mg (2.60 mmol) of previous compound Ac-Val-Ala-OH were suspended in 20 ml of anhydrous THF. 676 mg (2.74 mmol) of 2-Ethoxy-1-ethoxycarbonyl-1,2-dihydroquinoline (EEDQ) previously dissolved in 5 mL of anhydrous DMF was then into the flask and the turbid reaction mixture was stirred overnight at room temperature. Volatiles were then evaporated under reduced pressure and the crude residue was dry-loaded and purified by chromatography on silica gel (DCM/MeOH gradient from 99:1 to 85:15) to afford 809 mg (83%) of title compound as a white solid. ¹H NMR (300 MHZ, DMSO-d₆) δ 9.84 (s, 1H), 8.18 (d, J=7.0 Hz, 1H), 7.90 (d, J=8.6 Hz, 1H), 7.53 (d, J=8.6 Hz, 2H), 7.28 (d, J=8.6 Hz, 2H), 5.44 (d, J=4.4 Hz, 1H), 4.62 (q, J=6.2 Hz, 1H), 4.39 (p, J=7.6, 7.2 Hz, 1H), 4.17 (dd, J=8.5, 6.8 Hz, 1H), 2.70 (t, J=2.6 Hz, 1H), 1.96 (dt, J=13.2, 6.6 Hz, 1H), 1.88 (s, 3H), 1.30 (d, J=7.1 Hz, 3H), 0.86 (dd, J=10.9, 6.8 Hz, 6H). ESI⁺[M+H]⁺=374.2. HPLC Method 2 retention time=3.95 min.

### 1.2.3.3) Synthesis of 1-(4-((S)-2-((S)-2-acetamido-3-(4-nitrophenyl)methylbutanamido)propanamido)phenyl)but-3-yn-1-yl carbonate

94 mg (0.25 mmol) of (2S)-2-acetamido-N-((2S)-1-((4-(1-hydroxybut-3-yn-1-yl)phenyl)amino)-1-oxopropan-2-yl)-3-methylbutanamide and 153 mg (0.50 mmol) of bis(4-nitrophenyl) carbonate were dissolved in 2 mL of anhydrous DMF. 98 mg (0.76 mmol) of DIPEA were added and the reaction mixture was stirred overnight at room temperature. Volatiles were removed under reduced pressure and the crude residue was purified by chromatography on silica gel (DCM/MeOH gradient from 99:1 to 90:10) to afford 118 mg (87%) of title compound as a yellow solid. ¹H NMR (300 MHZ, DMSO-d₆) δ 9.99 (s, 1H), 8.35-8.26 (m, 2H), 8.22 (d, J=7.0 Hz, 1H), 7.89 (d, J=8.6 Hz, 1H), 7.63 (d, J=8.7 Hz, 2H), 7.58-7.48 (m, 2H), 7.43 (d, J=8.7 Hz, 2H), 5.74 (d, J=7.5 Hz, 1H), 4.39 (p, J=7.2 Hz, 1H), 4.17 (dd, J=8.5, 6.9 Hz, 1H), 3.01-2.84 (m, 3H), 1.99-1.91 (m, 1H), 1.88 (s, 3H), 1.31 (d, J=7.1 Hz, 3H), 0.86 (dd, J=11.2, 6.8 Hz, 6H). ESI⁺[M+H]⁺=539.1. HPLC Method 2 retention time=6.48 min.

### 1.2.3.4) Synthesis of Compound B5

44 mg (0.082 mmol) of previous compound 1-(4-((S)-2-((S)-2-acetamido-3-methylbutanamido)propanamido)phenyl)but-3-yn-1-yl (4-nitrophenyl) carbonate, 53 mg (0.074) of MMAE and 11.1 mg (0.082 mmol) of HOBt were dissolved in 1 mL of a 85:15 (v/v) mixture of anhydrous DMF/pyridine. 10.6 mg (0.082 mmol) of DIPEA was added. The reaction was stirred 16 hours at room temperature and was directly purified using HPLC preparative method 5 to afford 41 mg (50%) of compound B5 as a white solid. ESI⁺[M+Na]⁺=1139.7. HPLC Method 2 retention time=7.40 min.

### 1.2.3.5) Synthesis of Compound B6

Compound B6 was synthesized following the same procedures that were used for synthesis of compound B5 with slight adjustments. 1-(4-((S)-2-((S)-2-acetamido-3-methylbutanamido)propanamido)phenyl)but-3-yn-1-yl (4-nitrophenyl) carbonate coupling reaction with Exatecan Mesylate was conducted at 40° C. for 3 hours instead of overnight at room temperature. After removal of volatiles under reduced pressure, the reaction mixture was purified by chromatography on silica gel (DCM/MeOH gradient from 99:1 to 95:5) to afford 53.3 mg (78%) of compound B6 as a yellow/greenish solid. ESI⁺[M+H]⁺=835.3. HPLC Method 2 retention time=6.50 and 6.60 min (diastereoisomeric mixture).

### 1.2.4) Synthesis of Compound B7 and Compound B8

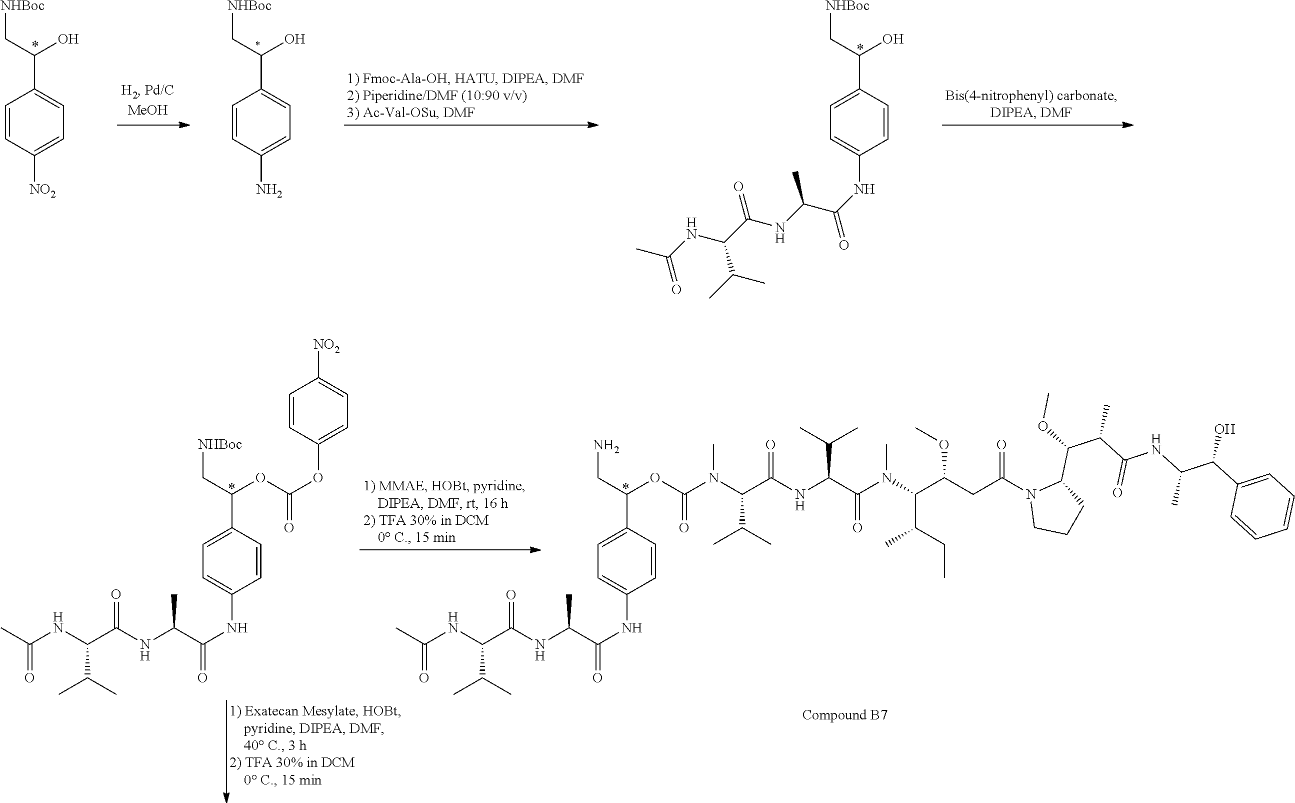

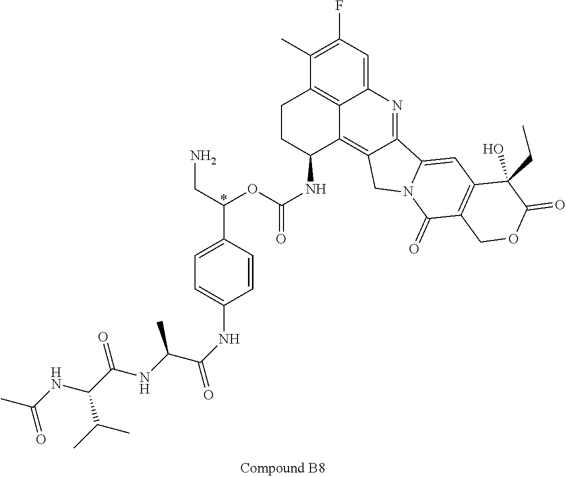 [SMILES: CC[C@@]1(O)C(=O)OCc2c1cc1n(c2=O)Cc2c-1nc1cc(F)c(C)c3c1c2[C@@H](NC(=O)OC(CN)c1ccc(NC(=O)[C@H](C)NC(=O)[C@@H](NC(C)=O)C(C)C)cc1)CC3]

### 1.2.4.1) Synthesis of tert-butyl (2-(4-aminophenyl)-2-hydroxyethyl)carbamate

A MeOH solution containing 911 mg (3.23 mmol) of commercially available tert-butyl (2-hydroxy-2-(4-nitrophenyl)ethyl) carbamate (CAS #939757-25-2) was transferred into a stainless steel hydrogenation reactor. After a first argon purge, a catalytic amount of 5% wt Pd/C was added. The reactor was then purged twice with H₂ and kept under a H₂ pressure of 10 bar for 5 hours at room temperature, while keeping the reaction stirred. After filtration of the reaction with a 0.45 μm PTFE filter and MeOH removal under vacuum, 749 mg (92%) of title compound was obtained as a white solid. ESI⁺[M+H]⁺=253.2. HPLC Method 2 retention time=2.73 min.

### 1.2.4.2) Synthesis of tert-butyl (2-(4-((S)-2-((S)-2-acetamido-3-methylbutanamido)propanamido)phenyl)-2-hydroxyethyl)carbamate

150 mg (0.60 mmol) of previous compound tert-butyl (2-(4-aminophenyl)-2-hydroxyethyl) carbamate, 222 mg (0.71 mmol) of Fmoc-Ala-OH and 81 mg (0.62 mmol) of DIPEA were dissolved in 5 mL of anhydrous DMF. 181 mg (0.71 mmol) of HATU is added and the reaction is stirred at room temperature overnight. Volatiles were then removed under reduced pressure and the crude residue was purified by chromatography on silica gel (DCM/MeOH gradient from 100:0 to 90:10) to quantitatively afford the first intermediate tert-butyl (2-(4-((S)-2-((((9H-fluoren-9-yl)methoxy)carbonyl)amino)propanamido)phenyl)-2-hydroxyethyl)carbamate that was directly engaged in Fmoc-deprotection. HPLC Method 2 retention time=7.7 min.

tert-butyl (2-(4-((S)-2-((((9H-fluoren-9-yl)methoxy)carbonyl)amino)propanamido)phenyl)-2-hydroxyethyl)carbamate was dissolved in 5 mL of DMF/piperidine 9:1 (v/v) and stirred 15 min at room temperature. Volatiles were then removed under reduced pressure and the crude residue was purified by chromatography on silica gel (DCM/MeOH gradient from 99:1 to 80:20) to afford 130 mg (68% over two steps) of the second intermediate tert-butyl (2-(4-((S)-2-aminopropanamido)phenyl)-2-hydroxyethyl)carbamate as a white foamy solid. HPLC Method 2 retention time=3.75 min.

130 mg (0.40 mmol) of tert-butyl (2-(4-((S)-2-aminopropanamido)phenyl)-2-hydroxyethyl) carbamate and 124 mg (0.48 mmol) of commercially available Ac-Val-OSu (CAS #56186-37-9) were dissolved in 3 mL of anhydrous DMF and the reaction mixture was stirred at room temperature overnight. Volatiles were then removed under reduced pressure and the crude residue was triturated with 3 mL of DCM to afford 95 mg (51%) of title compound as a white solid. ¹H NMR (300 MHZ, DMSO-d₆) δ 9.82 (s, 1H), 8.16 (d, J=7.1 Hz, 1H), 7.88 (d, J=8.6 Hz, 1H), 7.53 (d, J=8.4 Hz, 2H), 7.22 (d, J=8.5 Hz, 2H), 6.73-6.58 (m, 1H), 5.27 (d, J=4.4 Hz, 1H), 4.58-4.47 (m, 1H), 4.40 (q, J=7.1 Hz, 1H), 4.17 (dd, J=8.4, 6.8 Hz, 1H), 3.15-2.90 (m, 2H), 1.88 (s, 4H), 1.35 (s, 9H), 1.30 (d, J=7.1 Hz, 3H), 0.92-0.73 (m, 6H). ESI⁺[M+H]⁺=487.3. HPLC Method 2 retention time=4.50 min.

### 1.2.4.3) Synthesis of tert-butyl (2-(4-((S)-2-((S)-2-acetamido-3-methylbutanamido)propanamido)phenyl)-2-(((4-nitrophenoxy)carbonyl)oxy)ethyl)carbamate

286 mg (0.62 mmol) of tert-butyl (2-(4-((S)-2-((S)-2-acetamido-3-methylbutanamido)propanamido)phenyl)-2-hydroxyethyl)carbamate and 375 mg (1.23 mmol) of bis(4-nitrophenyl) carbonate were dissolved in 3 mL of anhydrous DMF. 318 mg (2.46 mmol) of DIPEA were added and the reaction mixture was stirred 3 hours at room temperature. Volatiles were removed under reduced pressure and the crude residue was purified by chromatography on silica gel (DCM/MeOH gradient from 99:1 to 90:10) to afford 336 mg (87%) of title compound as a yellow solid. ¹H NMR (300 MHz, DMSO-d₆) δ 9.99 (s, 1H), 8.36-8.26 (m, 2H), 8.22 (d, J=6.9 Hz, 1H), 7.89 (d, J=8.6 Hz, 1H), 7.63 (d, J=8.6 Hz, 2H), 7.56-7.46 (m, 2H), 7.34 (d, J=8.6 Hz, 2H), 7.22 (t, J=5.6 Hz, 1H), 5.68 (t, J=6.0 Hz, 1H), 4.38 (p, J=7.1 Hz, 1H), 4.17 (dd, J=8.5, 6.9 Hz, 1H), 3.49-3.34 (m, 2H), 2.01-1.90 (m, 1H), 1.87 (s, 3H), 1.37 (s, 9H), 1.30 (d, J=7.1 Hz, 3H), 0.86 (dd, J=11.1, 6.8 Hz, 6H). ESI⁺[M+Na]⁺=652.2. HPLC HPLC Method 2 retention time=7.13 min.

### 1.2.4.4) Synthesis of Compound B7

43.2 mg (0.069 mmol) of previous compound tert-butyl (2-(4-((S)-2-((S)-2-acetamido-3-methylbutanamido)propanamido)phenyl)-2-(((4-nitrophenoxy)carbonyl)oxy)ethyl)carbamate, 41 mg (0.057) of MMAE and 6.7 mg (0.057 mmol) of HOBt were dissolved in 1 mL of a 85:15 (v/v) mixture of anhydrous DMF/pyridine. 11.0 mg (0.086 mmol) of DIPEA was added. The reaction was stirred 16 hours at room temperature and volatiles were evaporated under reduced pressure. The crude residue was purified by chromatography on silica gel (DCM/MeOH gradient from 99:1 to 90:10) to afford 47 mg (55%) of intermediate compound (slightly yellow solid) that was directly engaged into the Boc-deprotection step. ESI⁺[M+Na]⁺=1230.7. HPLC Method 2 retention time=7.55 min.

The obtained solid was re-dissolved at 0° C. with a TFA/DCM (30:70 v/v) solution and stirred 15 minutes at room temperature. Volatiles were evaporated under reduced pressure, the crude residue was taken up in a water/ACN (1:1 v/v) solution and purified using HPLC preparative method 5 to afford 15 mg (30%) of compound B7 as a white solid. ESI⁺[M+H]⁺=1108.7. HPLC Method 2 retention time=5.8 and 5.9 min (diastereoisomeric mixture).

### 1.2.4.5) Synthesis of Compound B8

Compound B8 was synthesized following the same procedures that were used for synthesis of compound B7 with slight adjustments. tert-butyl (2-(4-((S)-2-((S)-2-acetamido-3-methylbutanamido)propanamido)phenyl)-2-(((4-nitrophenoxy)carbonyl)oxy)ethyl)carbamate coupling reaction with Exatecan Mesylate was conducted at 40° C. for 3 hours instead of overnight at room temperature.

69 mg (95%) of intermediate compound 1-(4-((S)-2-((S)-2-acetamido-3-methylbutanamido)propanamido)phenyl)-2-((tert-butoxycarbonyl)amino)ethyl ((1R,9R)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamate was obtained at a brown yellow solid. ESI⁺[M+H]⁺=926.4. HPLC Method 2 retention time=6.7 and 6.8 min (diastereoisomeric mixture).

Boc-deprotection was conducted as described for compound B7. Purification using HPLC preparative method 5 afforded 47 mg (70%) of compound B8 as a yellow solid. ESI⁺[M+H]⁺=826.4. HPLC Method 3 retention time=8.30 and 8.75 min (diastereoisomeric mixture).

### 1.2.4.6) Synthesis of Stereopure Compounds B8-S and B8-R

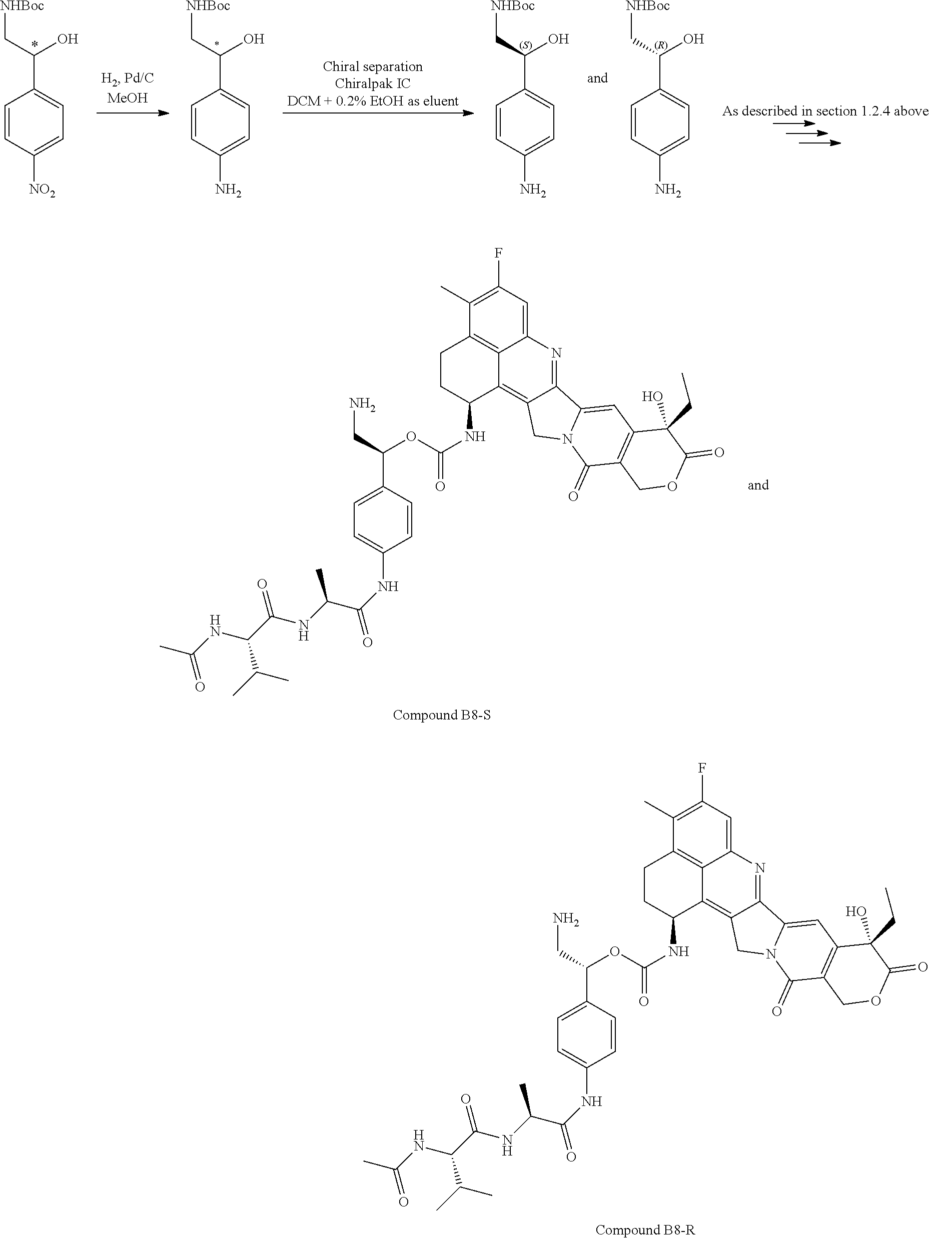

### 1.2.4.6.1) Chiral Separation of Racemic Mixture of tert-butyl (2-(4-aminophenyl)-2-hydroxyethyl)carbamate

Chiral separation of racemic tert-butyl (2-(4-aminophenyl)-2-hydroxyethyl)carbamate was IC MPLC column 30×100 mm, 20 μm (Daicel cat #83M73) on a Teledyne Isco CombiFlash® Rf200 system. Mobile phase was DCM+0.2% (v/v) EtOH (isocratic gradient). Flow rate was 12 mL/min. Sample solvent was DCM+0.2% (v/v) EtOH. Mass recovery of the two enantiomers after separation was above 75%.

tert-butyl (S)-(2-(4-aminophenyl)-2-hydroxyethyl)carbamate retention time was 21 min, whereas tert-butyl (R)-(2-(4-aminophenyl)-2-hydroxyethyl)carbamate retention time was 29 min. Absolute configuration of the enantiomers (previously dissolved in a 1:1 mixture of heptane/ethanol and allowed to slowly evaporate for 1 week to induce the formation of crystals) was confirmed by x-ray crystallography. A block-shaped crystal was mounted on a nylon loop in perfluoroether oil. Data were collected using a Xcalibur, Atlas, Gemini ultra-diffractometer equipped with an Oxford Cryosystems low-temperature device operating at T=150.00 (10) K. Data were measured using w scans using Cu K_{α} radiation. The structure was solved with the ShelXT solution program using dual methods and by using Olex2 (O. V. Dolomanov et al., Olex2: A complete structure solution, refinement and analysis program, J. Appl. Cryst., 2009, 42, 339-341) as the graphical interface. The model was refined with ShelXL 2018/3 (Sheldrick, G. M., Crystal structure refinement with ShelXL, Acta Cryst., 2015, C71, 3-8) using full matrix least squares minimization on F².

### 1.2.4.6.2) Synthesis of Stereopure B8-S and B8-R Compounds

Stereopure compounds B8-S and B8-R were synthesized as described in previous section 1.2.4, without any appreciable changes in reaction conditions, reactivity or overall yields.

Final purification using HPLC preparative method 5 afforded 54 mg of compound B8-S as a yellow solid. ESI⁺[M+H]⁺=826.4. HPLC Method 3 retention time=8.45 min.

Final purification using HPLC preparative method 5 afforded 46 mg of compound B8-R as a yellow solid. ESI⁺[M+H]⁺=826.4. HPLC Method 3 retention time=8.90 min.

### 1.2.5) Synthesis of Compound B9, Compound B10 and Compound B11

### 1.2.5.1) Synthesis of Compound B9

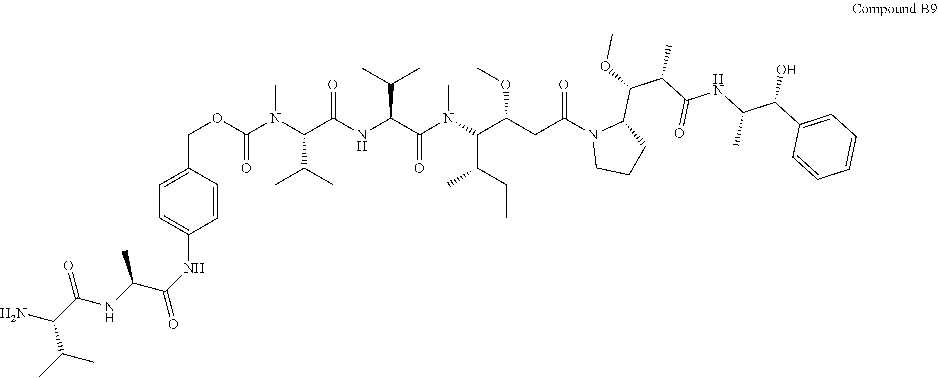

20.0 mg (0.036 mmol) of Boc-Val-Ala-PAB-PNP (CAS #1884578-00-0, Iris Biotech), 23.1 mg (0.032) of MMAE and 5 mg (0.036 mmol) of HOBt were dissolved in 1 ml of a 85:15 (v/v) mixture of anhydrous DMF/pyridine. 4.6 mg (0.036 mmol) of DIPEA was added. The reaction was stirred 16 hours at room temperature and volatiles were evaporated under reduced pressure. The crude residue was dissolved with 2 mL of a TFA/DCM (30:70 v/v) solution and stirred 1 hour at room temperature. Volatiles were evaporated under reduced pressure, the crude residue was taken up in a water/ACN (1:1 v/v) solution and purified using HPLC preparative method 5 to afford 11 mg (26%) of compound B9 as a white solid. ESI⁺[M+H]⁺=1037.7. HPLC Method 2 retention time=7.9 min.

### 1.2.5.2) Synthesis of Compound B10

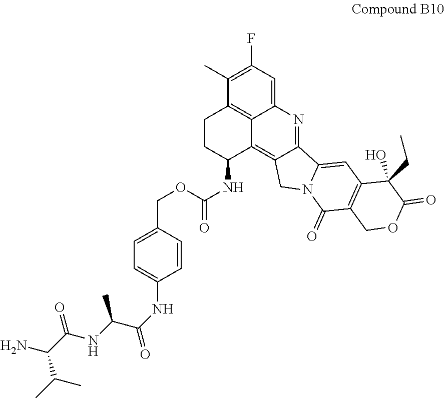 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](N)C(C)C)cc1)CC3]

Compound B10 was synthesized following the same procedures that were used for synthesis of compound B9 with slight adjustments. Boc-Val-Ala-PAB-PNP (CAS #1884578-00-0) coupling reaction with Exatecan Mesylate was conducted at 40° C. for 3 hours instead of overnight at room temperature.

Boc-deprotection was conducted as described for above compound B9. Purification using HPLC preparative method 5 afforded 52 mg (90% over two steps) of compound B10 as a yellow solid. ESI⁺[M+H]⁺=755.3. HPLC Method 2 retention time=5.70 min.

### 1.2.5.3) Synthesis of Compound B11

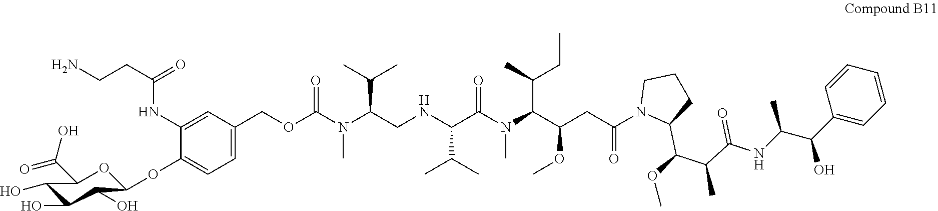

Compound (2S,3S,4S,5R,6S)-6-(2-(3-aminopropanamido)-4-((5R,8R,11R,12S)-11-((R)-sec-butyl)-12-(2-(2-((1R,2S)-3-(((1R,2S)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (compound B11) was synthesized as described in Jeffrey S C et al., Bioconjug. Chem., 2006, 17(3), 831-840).

### 1.3) Synthesis of Drug-Linkers

### 1.3.1) Synthesis of Glucuronide-Based Drug-Linkers

### 1.3.1.1) Synthesis of Compound DL1

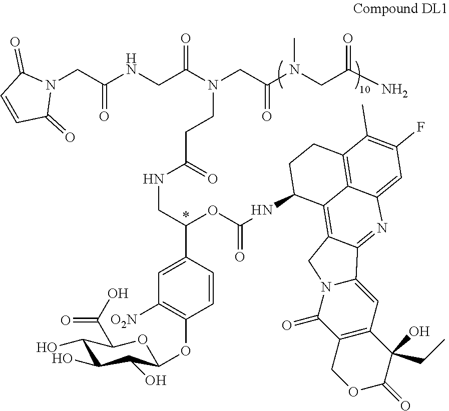

100 mg (0.081 mmol) of compound A1 (NHS-activated polysarcosine intermediate) and 51 mg (0.061 mmol) of compound B4 (NH₂-payload) were dissolved in anhydrous DMF in a small vial (0.080M concentration of compound B4). 41 mg (0.405 mmol) of triethylamine was added and the reaction was stirred 30 min at room temperature. After entire conversion of the reaction as observed by HPLC, piperidine is directly added into the reaction vial in order to reach a 8% (v/v) piperidine solution in DMF. The reaction is then stirred at room temperature 5-10 min, until entire Fmoc-deprotection is observed by HPLC. The reaction is slowly neutralized with a 10% TFA solution in water/ACN 1:1 (v/v) and purified using HPLC preparative method 5 to afford 74 mg (70% yield based on starting compound B4) of intermediate compound (2S,3S,4S,5R,6S)-6-(4-(41-amino-1-(((1R,9R)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)amino)-9-glycyl-12,15,18,21,24,27,30,33,36,39-decamethyl-1,6,11,14,17,20,23,26,29,32,35,38,41-tridecaoxo-2-oxa-5,9,12,15,18,21,24,27,30,33,36,39-dodecaazahentetracontan-3-yl)-2-nitrophenoxy)-3,4,5-trihydroxytetrahydro-2H-pyran-2-carboxylic acid as a yellow solid. ESI⁺[M+H]⁺=1731.7. HPLC Method 3 retention time=7.40 min and 7.70 min (equimolar diastereoisomeric mixture).

17.8 mg (0.010 mmol) of this compound and 2.85 mg (0.011 mmol) of maleimidoacetic acid N-hydroxysuccinimide ester were dissolved in anhydrous DMF (0.1M concentration of maleimide compound). 1.56 mg (0.015) of triethylamine was added and the reaction was stirred for 2 hours until entire conversion of the reaction as observed by HPLC. The reaction mixture is then diluted with a 1% TFA solution in water/ACN 1:1 (v/v) and purified using HPLC preparative method 6 to afford 13.7 mg (72%) of compound DL1 as a yellow solid. HRMS m/z (ESI⁺): Calc [M+2H]²⁺=934.8559; Exp [M+2H]²⁺=934.8544; Error=1.7 ppm. HPLC Method 3 retention time=7.73 min and 8.08 min (equimolar diastereoisomeric mixture).

### 1.3.1.2) Synthesis of Compound DL2

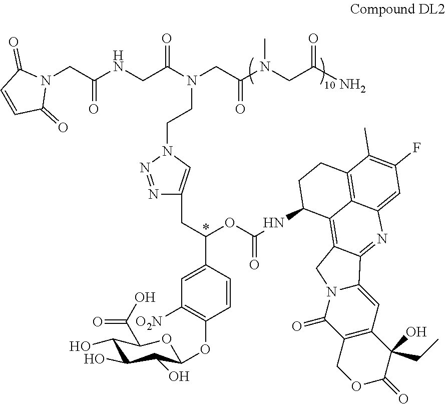

44.5 mg (0.049 mmol) of compound A2 (azide-polysarcosine intermediate) and 27.5 (0.033 mmol) of compound B2 (alkyne-payload) were dissolved in a 1:1 (v/v) solution of 100 mM PBS (pH=7.5) and DMSO, in order to reach a 0.060M concentration of compound B2. Freshly prepared CuSO₄ pentahydrate and sodium ascorbate solutions (approximately 250 mg/mL) were then sequentially added into the reaction vial in order to reach 0.08 molar equivalent Cu and 1 molar equivalent of sodium ascorbate (based on compound B2 molar equivalent in the reaction mixture). The reaction is purged with argon and stirred at 40° C. The reaction is monitored by HPLC and was complete in less than 2 hours. The reaction mixture is then diluted with a 0.1% TFA solution in water/ACN 1:1 (v/v) and purified using HPLC preparative method 5 to afford 44 mg (77% based on starting compound B2) of intermediate compound (2S,3S,4S,5R,6S)-6-(4-(2-(1-(35-amino-3-glycyl-6,9,12,15,18,21,24,27,30,33-decamethyl-5,8,11,14,17,20,23,26,29,32,35-undecaoxo-3,6,9,12,15,18,21,24,27,30,33-undecaazapentatriacontyl)-1H-1,2,3-triazol-4-yl)-1-((((1R,9R)-9-ethyl-5-fluoro-9-hydroxy-4-methyl-10,13-dioxo-2,3,9,10,13,15-hexahydro-1H, 12H-benzo[de]pyrano[3′,4′:6,7]indolizino[1,2-b]quinolin-1-yl)carbamoyl)oxy)ethyl)-2-nitrophenoxy)-3,4,5-trihydroxytetrahydro-2H-pyran-2-carboxylic acid as a yellow solid. ESI⁺[M+H]⁺=1755.7. HPLC Method 3 retention time=7.51 min and 7.82 min (diastereoisomeric mixture).

26.0 mg (0.015 mmol) of this compound and 4.11 mg (0.016 mmol) of maleimidoacetic acid N-hydroxysuccinimide ester were dissolved in anhydrous DMF (0.1M concentration of maleimide compound). 2.25 mg (0.022 mmol) of triethylamine was added and the reaction was stirred for 2 hours until entire conversion of the reaction was observed by HPLC. The reaction mixture was then diluted with a 1% TFA solution in water/ACN 1:1 (v/v) and purified using HPLC preparative method 6 to afford 16.0 mg (57%) of compound DL2 as a yellow solid. ESI⁺[M+H]⁺=1892.7. HPLC Method 3 retention time=7.95 min and 8.20 min (equimolar diastereoisomeric mixture).

### 1.3.1.3) Synthesis of Compound DL3

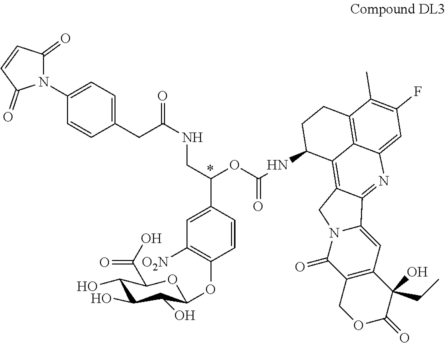

5.4 mg (0.023 mmol) of commercial 2-(4-(2,5-dioxo-2H-pyrrol-1(5H)-yl)phenyl)acetic acid (CAS #91574-45-7) and 9.04 mg (0.021 mmol) of COMU were dissolved in anhydrous DMF (0.1M concentration of maleimide compound). 4.75 mg (0.067 mmol) of triethylamine was added. The reaction was pre-incubated 2 minutes at room temperature and was transferred onto 9.8 mg (0.012 mmol) of compound B4 (pre-weighted in a reaction vial). The reaction was stirred for 30 min until entire conversion of the reaction was observed by HPLC. The reaction mixture was then diluted with a 1% TFA solution in water/ACN 1:1 (v/v) and purified using HPLC preparative method 5 to afford 4.7 mg (40%) of compound DL3 as a yellow solid. HRMS m/z (ESI⁺): Calc [M+H]⁺=1049.2847; Exp [M+H]⁺=1049.2873; Error=−2.5 ppm. HPLC Method 3 retention time=9.80 min (equimolar diastereoisomeric mixture).

### 1.3.1.4) Synthesis of Compound DL4

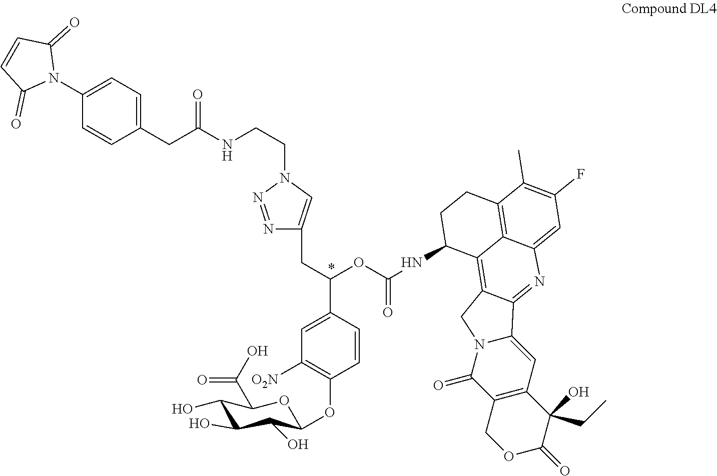

16.0 mg (0.054 mmol) of N-(2-azidoethyl)-2-(4-(2,5-dioxo-2,5-dihydro-1H-pyrrol-1-yl)phenyl)acetamide (synthesized following procedures already described in patent WO2019081455—quite unstable compound that can be kept only a few days upon storage at −20° C.) and 22.6 (0.027 mmol) of compound B2 were dissolved in a 1:1 (v/v) solution of 100 mM PBS (pH=7.5) and DMSO, in order to reach a 0.060M concentration of compound B2. Freshly prepared CuSO₄ pentahydrate and sodium ascorbate solutions (approximately 250 mg/mL) were then sequentially added into the reaction vial in order to reach 0.08 molar equivalent Cu and 1 molar equivalent of sodium ascorbate (based on compound B2 molar equivalent in the reaction mixture). The reaction was purged with argon and stirred at room temperature. The reaction was monitored by HPLC and was complete in less than 1 hour. The reaction mixture is then diluted with a 0.1% TFA solution in water/ACN 1:1 (v/v) and purified using HPLC preparative method 6 to afford 16 mg (53%) of compound DL4 as a yellow solid. HRMS m/z (ESI⁺): Calc [M+H]⁺=1144.3331; Exp [M+H]⁺=1144.3351; Error=−1.8 ppm. HPLC Method 3 retention time=9.61 min (equimolar diastereoisomeric mixture).

### 1.3.1.5) Synthesis of Compound DL5

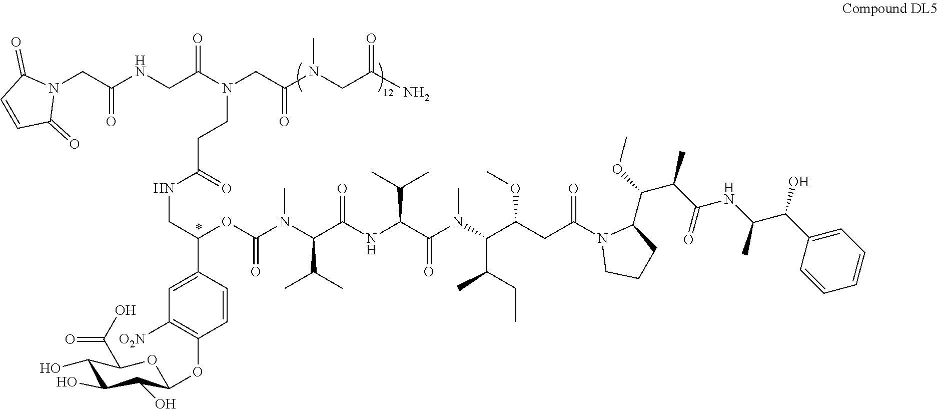

Compound DL5 was synthesized as described above, using the same procedure that was used for compound DL1. Starting materials were compound A4 (NHS-activated polysarcosine intermediate) and compound B3 (NH₂-payload).

10.8 mg (48% of two steps) of compound DL5 was obtained as a slightly yellow solid. HRMS m/z (ESI⁺): Calc [M+3H]³⁺=765.0460; Exp [M+3H]³⁺=765.0443; Error=2.2 ppm. HPLC Method 3 retention time=9.14 min (equimolar diastereoisomeric mixture).

### 1.3.1.6) Synthesis of Compound DL6

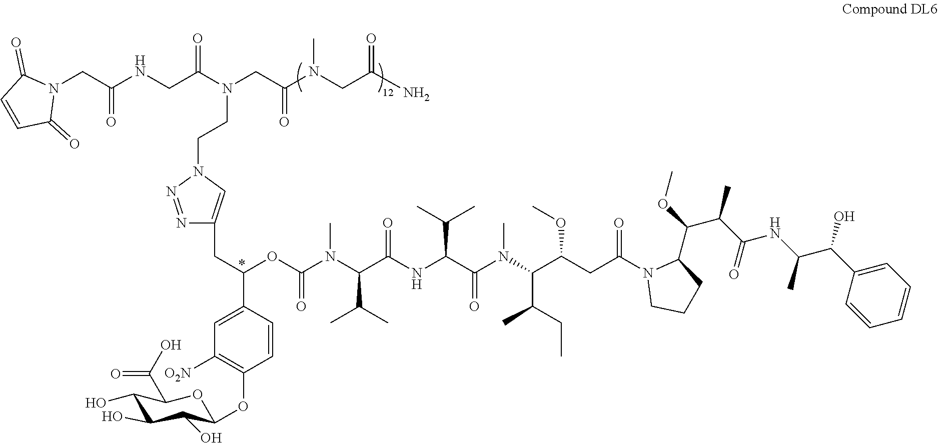

Compound DL6 was synthesized as described above, using the same procedure that was used for compound DL2. Starting materials were compound A5 (azide-polysarcosine intermediate) and compound B1 (alkyne-payload).

16.3 mg (41% of two steps) of compound DL6 was obtained as a slightly yellow solid. ESI⁺[M+2H]²⁺=1159.1. HPLC Method 3 retention time=9.32 min and 9.45 min (equimolar diastereoisomeric mixture).

### 1.3.1.7) Synthesis of Compound DL7

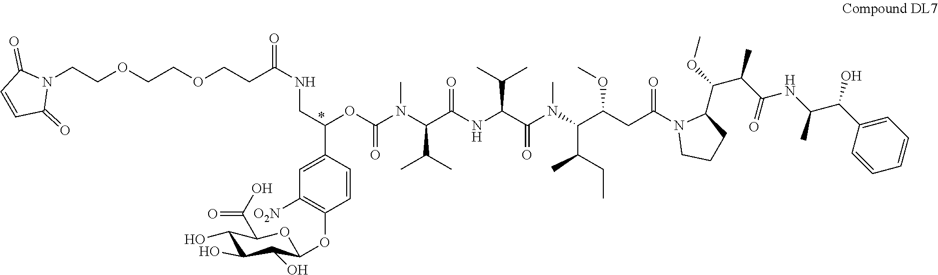

Compound DL7 was synthesized as described above, using the same procedure that was used for compound DL3. Starting materials were commercial compound maleimide-PEG₂-acid (CAS #1374666-32-6) and compound B3 (NH₂-payload).

15.6 mg (69%) of compound DL7 was obtained as a white solid. ESI⁺[M+2Na]²⁺=701.3. HPLC Method 3 retention time=10.94 min (equimolar diastereoisomeric mixture).

### 1.3.1.8) Synthesis of Compound DL8

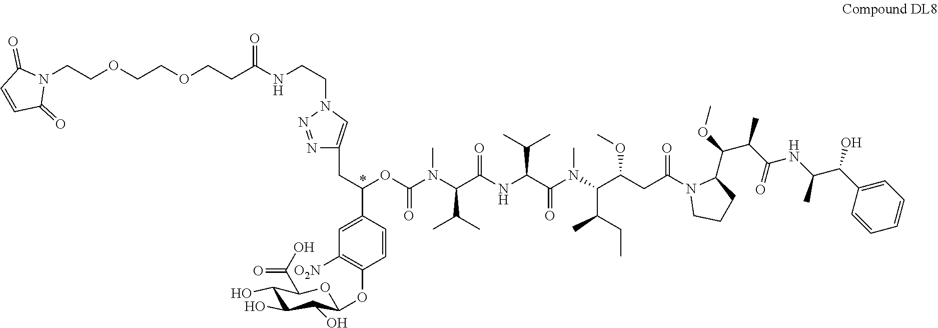

15.0 mg (0.013 mmol) of compound B1 (alkyne-payload) and 3.44 mg (0.040 mmol) of 2-azidoethylamine were dissolved in a 1:1 (v/v) solution of 100 mM PBS (pH=7.5) and DMSO, in order to reach a 0.060M concentration of compound B1. Freshly prepared CuSO₄ pentahydrate and sodium ascorbate solutions (approximately 250 mg/mL) were then sequentially added into the reaction vial in order to reach 0.08 molar equivalent Cu and 1 molar equivalent of sodium ascorbate (based on compound B1 molar equivalent in the reaction mixture). The reaction is purged with argon and stirred at room temperature. The reaction is monitored by HPLC and was complete in less than 1 hour. The reaction mixture is then diluted with a 0.1% TFA solution in water/ACN 1:1 (v/v) and purified using HPLC preparative method 5 to afford 18.2 mg (110% based on starting compound B1) of intermediate compound (2S,3S,4S,5R,6S)-6-(4-((3S,4R,7R,10R)-15-(1-(2-aminoethyl)-1H-1,2,3-triazol-4-yl)-4-((R)-sec-butyl)-3-(2-(2-((1S,2S)-3-(((1R,2S)-1-hydroxy-1-phenylpropan-2-yl)amino)-1-methoxy-2-methyl-3-oxopropyl)pyrrolidin-1-yl)-2-oxoethyl)-7,10-diisopropyl-5,11-dimethyl-6,9,12-trioxo-2,13-dioxa-5,8,11-triazapentadecan-14-yl)-2-nitrophenoxy)-3,4,5-trihydroxytetrahydro-2H-pyran-2-carboxylic acid as a white solid. ESI⁺[M+H]⁺=1213.6. HPLC Method 2 retention time=5.55 min and 5.65 min (diastereoisomeric mixture).

5.79 mg (0.022 mmol) of commercial maleimide-PEG₂-acid (CAS #1374666-32-6) and 9.00 mg (0.021 mmol) of COMU were dissolved in anhydrous DMF (0.1M concentration of maleimide compound). 6.1 mg (0.060 mmol) of triethylamine was added. The reaction was pre-incubated 2 minutes at room temperature and was transferred onto 18.2 mg (0.012 mmol) of previous compound (pre-weighted in a reaction vial). The reaction was stirred for 30 min until entire conversion of the reaction was observed by HPLC. The reaction mixture was then diluted with a 1% TFA solution in water/ACN 1:1 (v/v) and purified using HPLC preparative method 5 to afford 16.0 mg (73%) of compound DL8 as a white solid. HRMS m/z (ESI⁺): Calc [M+2H]²⁺=726.8609; Exp [M+2H]²⁺=726.8631; Error=−3.1 ppm. HPLC Method 3 retention time=10.40 min and 10.56 min (equimolar diastereoisomeric mixture).

### 1.3.1.9) Synthesis of Compound DL9

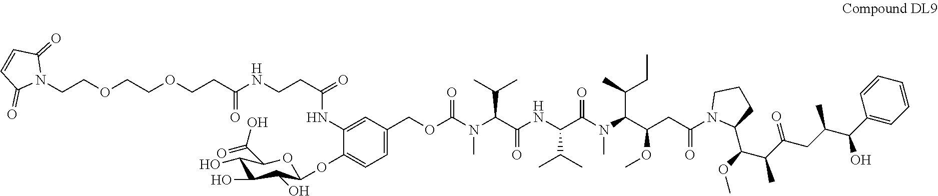

Compound DL9 was synthesized as described above, using the same procedure that was used for compound DL3. Starting materials were commercial compound maleimide-PEG₂-acid (CAS #1374666-32-6) and compound B11 (NH₂-payload).

10.0 mg (41%) of compound DL9 was obtained as a white solid. HRMS m/z (ESI⁺): Calc [M+2H]²⁺=685.3549; Exp [M+2H]²⁺=685.3554; Error=−0.8 ppm. HPLC Method 3 retention time=11.38 min.

### 1.3.1.10) Synthesis of Compound DL10

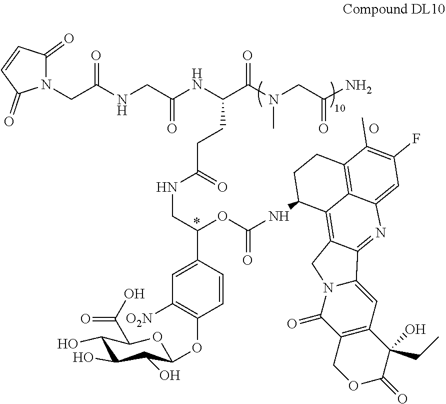

Compound DL10 was synthesized as described above, using the same procedure that was used for compound DL1. Starting materials were compound A6 (NHS-activated polysarcosine intermediate) and compound B4 (NH₂-payload).

54.1 mg (44% over two steps) of compound DL10 was obtained as a yellow solid. HRMS m/z (ESI⁺): Calc [M+2H]²⁺=934.8559; Exp [M+2H]²⁺=934.8545; Error=1.5 ppm. HPLC Method 3 retention time=7.68 min and 8.01 min (equimolar diastereoisomeric mixture).

### 1.2.1.11) Synthesis of Compounds DL10-S Et DL10-R

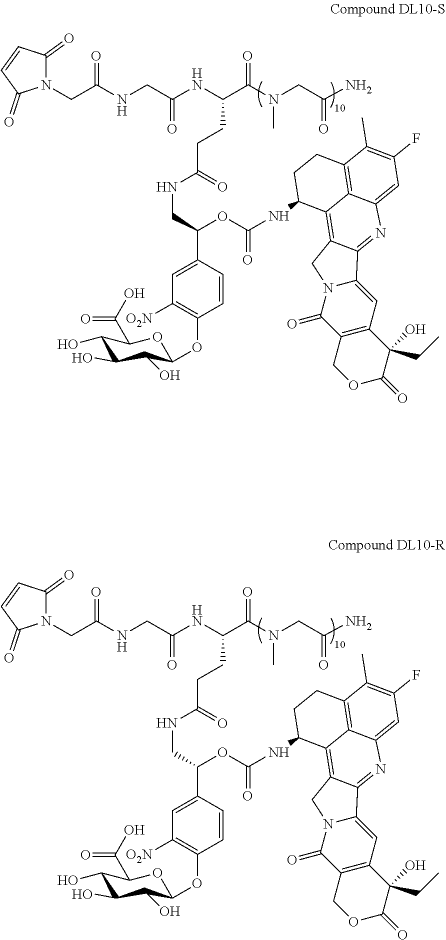

Compound DL10-S and DL10-R were synthesized as described above, using the same procedure that was used for compound DL1. Starting materials were respectively compound B4-S and B4-R (NH₂-payload) and compound A6 (NHS-activated polysarcosine intermediate).

4.7 mg of compound DL10-S was obtained as a yellow solid. ESI⁺[M+Na]⁺=1890.7. HPLC Method 3 retention time=7.62 min.

4.0 mg of compound DL10-R was obtained as a yellow solid. ESI⁺[M+Na]⁺=1890.7. HPLC Method 3 retention time=8.06 min.

### 1.2.1.12) Synthesis of Compound DL11

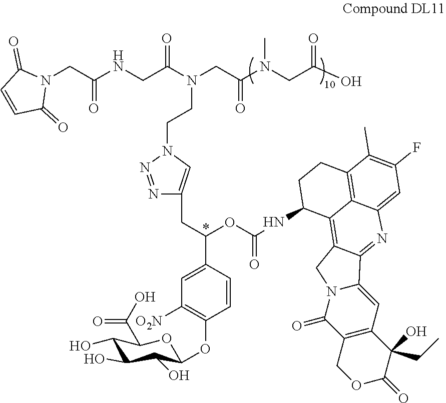

Compound DL11 was synthesized as described above, using the same procedure that was used for compound DL2. Starting materials were compound A3 (azide-polysarcosine intermediate) and compound B2 (alkyne-payload).

14.3 mg (43% over two steps) of compound DL11 was obtained as a yellow solid. HRMS m/z (ESI⁺): Calc [M+2H]²⁺=947.3536; Exp [M+2H]²⁺=947.3540; Error=−0.5 ppm. HPLC Method 3 retention time=8.13 min and 8.36 min (equimolar diastereoisomeric mixture).

### 1.3.2) Synthesis of Dipeptide-Based Drug-Linkers

### 1.3.2.1) Synthesis of Compound DL12

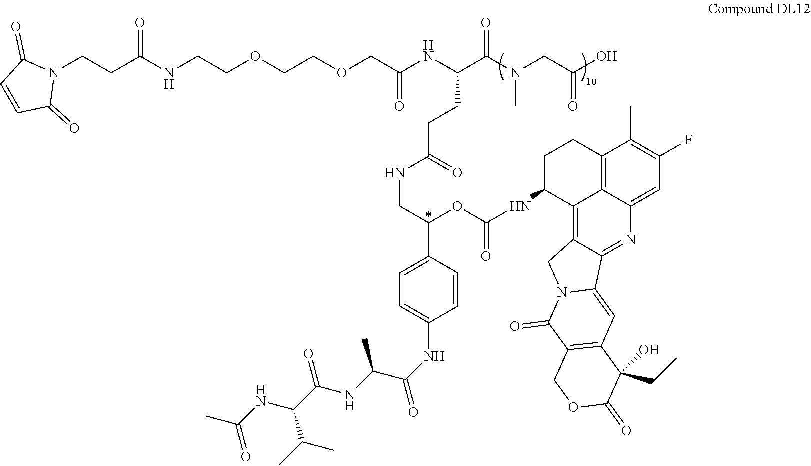

Compound DL12 was synthesized as described above, using the same procedure that was used for compound DL1. Starting materials were compound A7 (NHS-activated polysarcosine intermediate) and compound B8 (NH₂-payload).

34.9 mg (46% over two steps) of compound DL12 was obtained as a yellow solid. HRMS m/z (ESI⁺): Calc [M+2H]²⁺=981.4394; Exp [M+2H]²⁺=981.4398; Error=−0.4 ppm. HPLC Method 3 retention time=8.81 min and 8.94 min (equimolar diastereoisomeric mixture).

### 1.3.2.2) Synthesis of Compound DL12-S and DL12-R

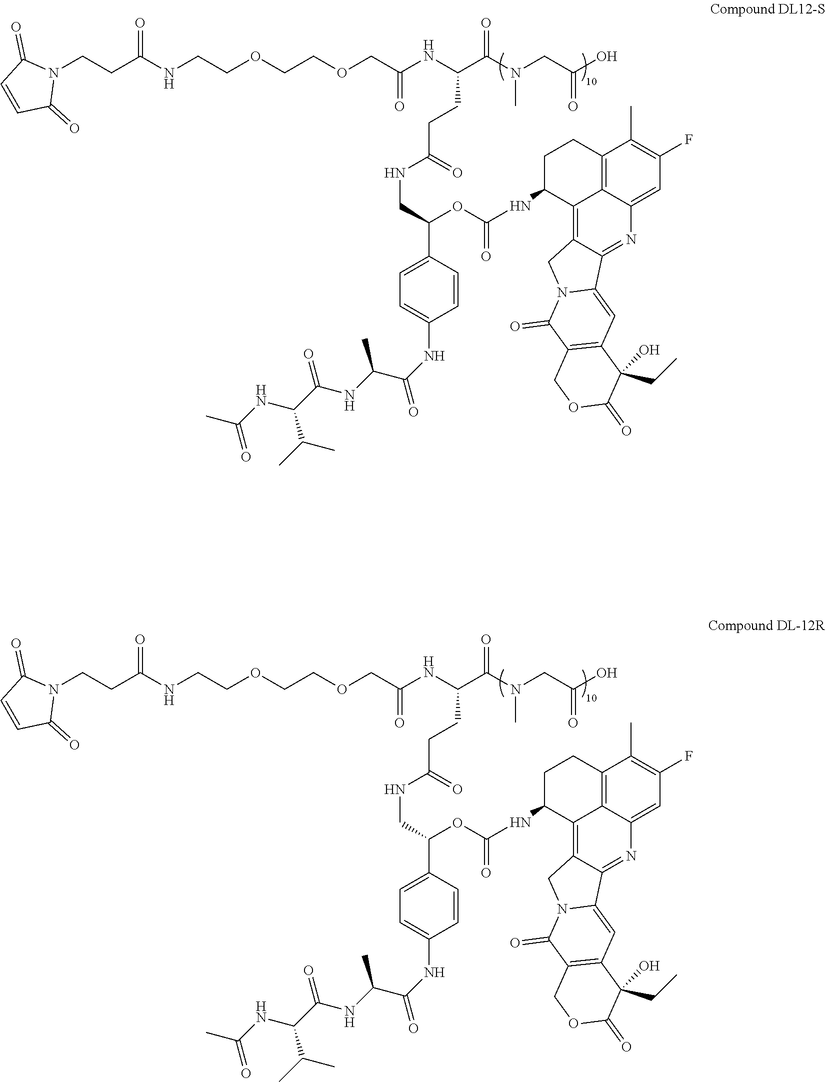

Compound DL12-S and DL12-R were synthesized as described above, using the same procedure that was used for compound DL1. Starting materials were respectively compound B8-S and B8-R (NH₂-payload) and compound A7 (NHS-activated polysarcosine intermediate).

18.1 mg of compound DL12-S was obtained as a yellow solid. ESI⁺[M+2H]²⁺=981.4. HPLC Method 3 retention time=8.78 min.

16.3 mg of compound DL12-R was obtained as a yellow solid. ESI⁺[M+2H]²⁺=981.4. HPLC Method 3 retention time=8.96 min.

### 1.3.2.3) Synthesis of Compound DL13

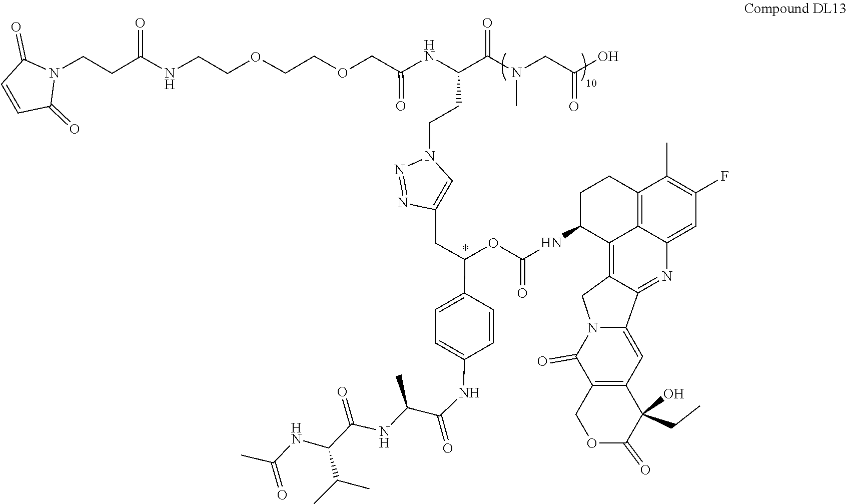

Compound DL13 was synthesized as described above, using the same procedure that was used for compound DL2. Starting materials were compound A8 (azide-polysarcosine intermediate) and compound B6 (alkyne-payload).

4.5 mg (24% over two steps) of compound DL13 was obtained as a yellow solid. HRMS m/z (ESI⁺): Calc [M+2H]²⁺=993.4451; Exp [M+2H]²⁺=993.4416; Error=3.5 ppm. HPLC Method 3 retention time=8.88 min and 9.11 min (equimolar diastereoisomeric mixture).

### 1.3.2.4) Synthesis of Compound DL14

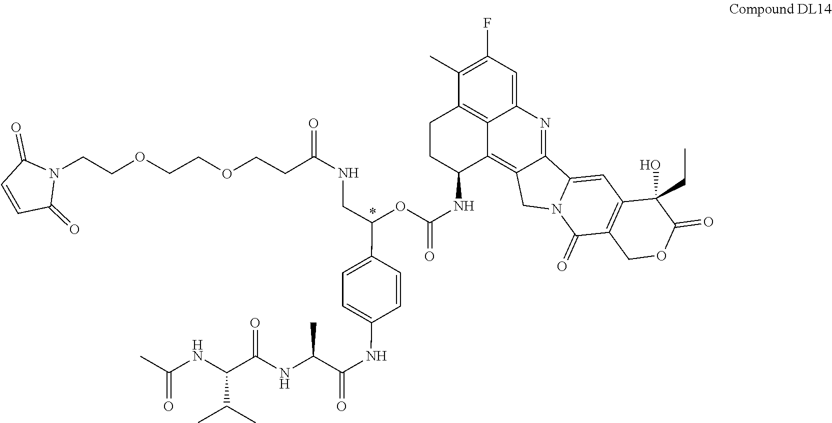 [SMILES: CC[C@@]1(O)C(=O)OCc2c1cc1n(c2=O)Cc2c-1nc1cc(F)c(C)c3c1c2[C@@H](NC(=O)OC(CNC(=O)CCOCCOCCN1C(=O)C=CC1=O)c1ccc(NC(=O)[C@H](C)NC(=O)[C@@H](NC(C)=O)C(C)C)cc1)CC3]

Compound DL14 was synthesized as described above, using the same procedure that was used for compound DL3. Starting materials were commercial compound maleimide-PEG₂-acid (CAS #1374666-32-6) and compound B8 (NH₂-payload).

7.0 mg (59%) of compound DL14 was obtained as a yellow solid. HRMS m/z (ESI⁺): Calc [M+H]⁺=1065.4364; Exp [M+H]⁺=1065.4370; Error=−0.5 ppm. HPLC Method 3 retention time=10.20 min and 10.44 min (equimolar diastereoisomeric mixture).

### 1.3.2.5) Synthesis of Compound DL15

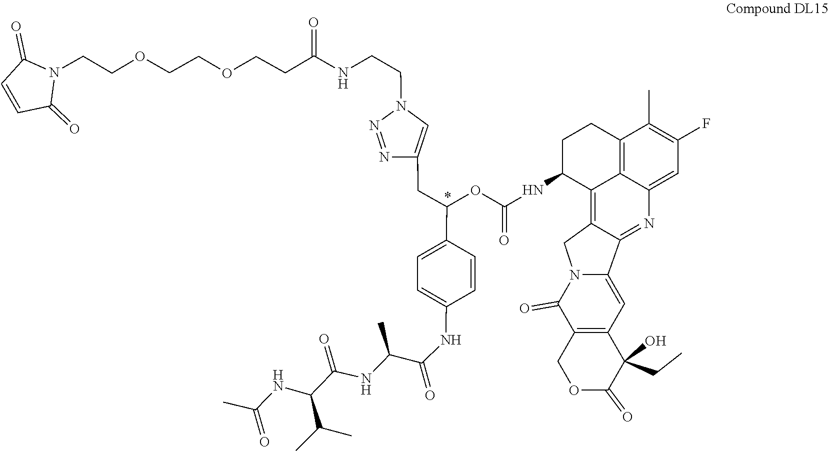 [SMILES: CC[C@]1(O)C(=O)OCc2c1cc1n(c2=O)Cc2c-1nc1cc(F)c(C)c3c1c2[C@@H](NC(=O)OC(Cc1cn(CCNC(=O)CCOCCOCCN2C(=O)C=CC2=O)nn1)c1ccc(NC(=O)[C@H](C)NC(=O)[C@H](NC(C)=O)C(C)C)cc1)CC3]

Compound DL15 was synthesized as described above, using the same procedure that was used for compound DL8.

3.1 mg (20% over two steps) of compound DL15 was obtained as a yellow solid. HRMS m/z (ESI⁺): Calc [M+H]⁺=1160.4848; Exp [M+H]⁺=1160.4854; Error=−0.6 ppm. HPLC Method 3 retention time=9.96 min and 10.12 min (equimolar diastereoisomeric mixture).

### 1.3.2.6) Synthesis of Compound DL16

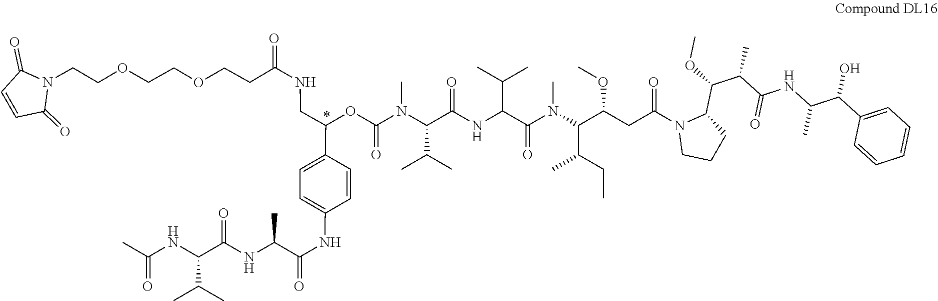

Compound DL16 was synthesized as described above, using the same procedure that was used for compound DL3. Starting materials were commercial compound maleimide-PEG₂-acid (CAS #1374666-32-6) and compound B7 (NH₂-payload).

5.3 mg (41%) of compound DL16 was obtained as a white solid. HRMS m/z (ESI⁺): Calc [M+Na]⁺=1369.7618; Exp [M+Na]⁺=1369.7621; Error=−0.3 ppm. HPLC Method 3 retention time=11.38 min (equimolar diastereoisomeric mixture).

### 1.3.2.7) Synthesis of Compound DL17

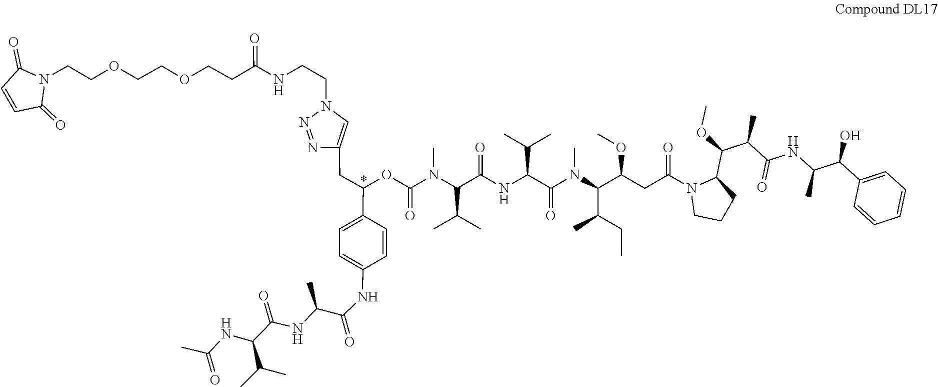

Compound DL17 was synthesized as described above, using the same procedure that was used for compound DL8.

8.9 mg (24% over two steps) of compound DL17 was obtained as a white solid. HRMS m/z (ESI⁺): Calc [M+H]⁺=1442.8294; Exp [M+H]⁺=1442.8284; Error=0.7 ppm. HPLC Method 3 retention time=11.81 min (equimolar diastereoisomeric mixture).

### 1.3.2.8) Synthesis of Compound DL18

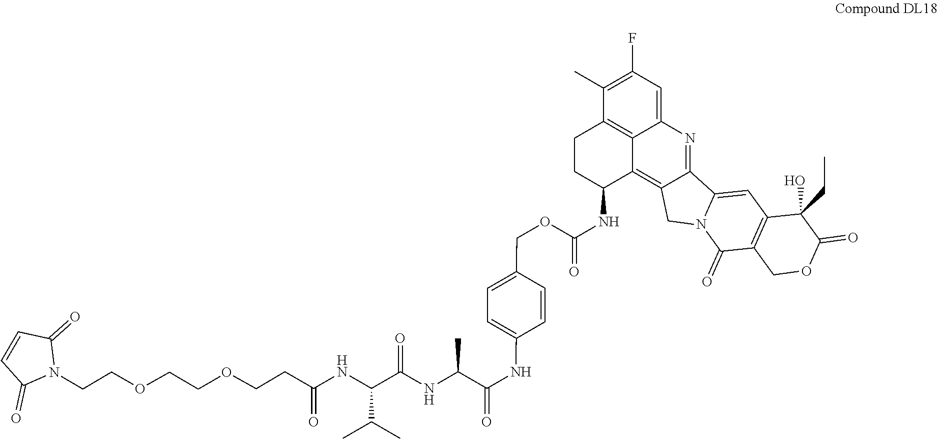 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CCOCCOCCN2C(=O)C=CC2=O)C(C)C)cc1)CC3]

Compound DL18 was synthesized as described above, using the same procedure that was used for compound DL3. Starting materials were commercial compound maleimide-PEG₂-acid (CAS #1374666-32-6) and compound B10 (NH₂-payload).

14.5 mg (62%) of compound DL18 was obtained as a yellow solid. ESI⁺[M+H]⁺=994.4. HPLC Method 3 retention time=11.62 min.

### 1.3.2.9) Synthesis of Compound DL19

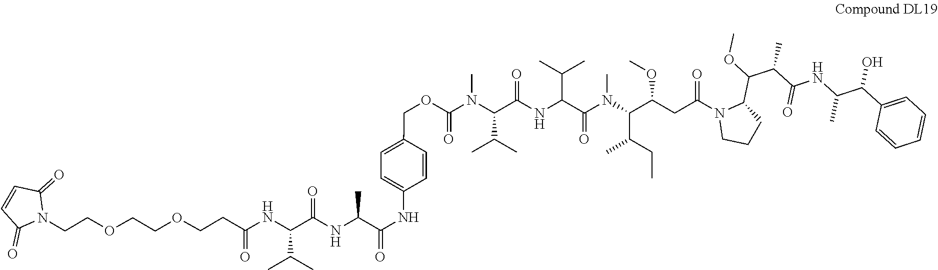 [SMILES: CC[C@H](C)[C@@H]([C@@H](CC(=O)N1CCC[C@H]1C(OC)[C@H](C)C(=O)N[C@@H](C)[C@H](O)c1ccccc1)OC)N(C)C(=O)C(NC(=O)[C@H](C(C)C)N(C)C(=O)OCc1ccc(NC(=O)[C@H](C)NC(=O)[C@@H](NC(=O)CCOCCOCCN2C(=O)C=CC2=O)C(C)C)cc1)C(C)C]

Compound DL19 was synthesized as described above, using the same procedure that was used for compound DL3. Starting materials were commercial compound maleimide-PEG₂-acid (CAS #1374666-32-6) and compound B9 (NH₂-payload).

4.7 mg (35%) of compound DL19 was obtained as a white solid. HRMS m/z (ESI⁺): Calc [M+H]⁺=1276.7439; Exp [M+H]⁺=1276.7441; Error=−0.1 ppm. HPLC Method 3 retention time=13.28 min.

### 1.3.2.10) Synthesis of Compound DL20

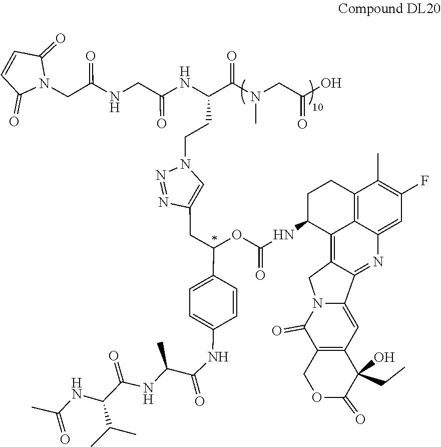

Compound DL20 was synthesized as described above, using the same procedure that was used for compound DL2. Starting materials were compound A3 (azide-polysarcosine intermediate) and compound B6 (alkyne-payload).

14.0 mg (36% over two steps) of compound DL20 was obtained as a yellow solid. ESI⁺[M+H]⁺=1883.8. HPLC Method 3 retention time=8.82 min and 9.07 min (equimolar diastereoisomeric mixture).

### 2) Preparation and Characterization of Conjugates

### 2.1) Preparation of Antibody-Drug Conjugates

A solution of antibody (10 mg/ml in PBS 7.4+1 mM EDTA) was treated with 14 molar equivalent of tris(2-carboxyethyl)phosphine (TCEP) for 2 hours at 37° C. The fully reduced antibody was buffer-exchanged with potassium phosphate 100 mM pH 7.4+1 mM EDTA by three rounds of dilution/centrifugation using Amicon 30K centrifugal filters device (Millipore). 10-12 molar equivalents of drug-linker (from a 12 mM DMSO stock solution) was added to the antibody (residual DMSO<10% v/v) in order to reach a drug-antibody ratio (DAR) of 8. The solution was incubated 30 min at room temperature. The conjugate was buffer-exchanged/purified with PBS pH 7.4 by four rounds of dilution/centrifugation using Amicon 30K centrifugal filters device and were sterile-filtered (0.20 μm PES filter).

Conjugates incorporating self-hydrolysable maleimides (maleimide-phenyl and maleimide-glycine) were incubated at 5 mg/mL in PBS 8.0 at 37° C. for 24 hours to ensure complete hydrolysis of the succinimidyl moiety, where buffer exchanged with PBS pH 7.4 using Amicon 30K centrifugal filters device and were sterile-filtered (0.20 μm PES filter).

Final protein concentration was assessed spectrophotometrically at 280 nm using a Colibri microvolume spectrometer device (Titertek Berthold).

### 2.2) Characterization of Conjugates

The resulting conjugates were characterized as follows:

### Reverse Phase Liquid Chromatography-Mass Spectrometry (RPLC-MS):

Denaturing RPLC-QTOF analysis was performed using the UHPLC method 4 described above. Briefly, conjugates were eluted on an Agilent PLRP-S 1000 Å 2.1×150 mm 8 μm (80° C.) using a mobile phase gradient of water/acetonitrile+0.1% formic acid (0.4 mL/min) and detected using a Bruker Impact II™ Q-TOF mass spectrometer scanning the 500-3500 m/z range (ESI⁺). Data were deconvoluted using the MaxEnt algorithm included in the Bruker Compass® software.

### Size Exclusion Chromatography (SEC):

SEC was performed on an Agilent 1100 HPLC system having an extra-column volume below 15 μL (equipped with short sections of 0.12 mm internal diameter peek tubing and a micro-volume UV flow cell). Column was an Agilent AdvanceBioSEC 300 Å 4.6×150 mm 2.7 μm (maintained at 30° C.). Mobile phase was 100 mM sodium phosphate and 200 mM sodium chloride (pH 6.8). 10% acetonitrile (v/v) was added to the mobile phase to minimize secondary hydrophobic interactions with the stationary phase and prevent bacterial growth. Flow rate was 0.35 mL/min. UV detection was monitored at 280 nm.

### Hydrophobic Interaction Chromatography (HIC):

Hydrophobic interaction chromatography (HIC) was performed on an Agilent 1100 HPLC system. Column was a Tosoh TSK-GEL BUTYL-NPR 4.6×35 mm 2.5 μm (25° ° C. Mobile phase A was 1.5 M (NH₄)₂SO₄₊₂₅ mM potassium phosphate pH 7.0. Mobile phase B was 25 mM potassium phosphate pH 7.0+15% isopropanol (v/v). Linear gradient was 0% B to 100% B in 10 min, followed by a 3 min hold at 100% B. Flow rate was 0.75 mL/min. UV detection was monitored at 220 and 280 nm.

### 2.3) Overview of Synthesized Conjugates

Antibody-drug conjugates exhibited one LC-1d (light chain with 1 drug-linker attached) and one HC-3d (heavy chain with 3 drug-linkers attached) absorbance peaks on their denaturing RPLC chromatogram (DAR8 conjugates). For mass spectrometry analysis of the heavy chain, the major glycoform was reported (G0F for trastuzumab).

| | | | | | | | | |
|---|---|---|---|---|---|---|---|---|
| | | | | | Orthogonal | | | |
| | | | | | hydrophobicity | Deconvoluted | Deconvoluted | Monomeric |
| Name of the | Drug- | Cleavable | | | masking | LC-1d | HC-3d | purity |
| conjugate | linker | modality | Ligand | Drug | entity | (Da) | (Da) | (%) |

Glycosidase-based conjugates

| | | | | | | | | |
|---|---|---|---|---|---|---|---|---|
| ADC101 | DL1 | Amide-linked | Proprietary | Exatecan | Polysarcosine | Calc: 25885; | Calc: 56294; | 95%+ |
| (invention) | | glucuronide | mAb1 | | 10 | Obs: 25885 | Obs: 56294 | |
| | | trigger | | | | | | |
| ADC102 | DL2 | Triazole- | Proprietary | Exatecan | Polysarcosine | Calc: 25909; | Calc: 56366; | 95%+ |
| (comparative) | | linked | mAb1 | | 10 | Obs: 25909 | Obs: 56367 | |
| | | glucuronide | | | | | | |
| | | trigger | | | | | | |
| ADC103 | DL3 | Amide-linked | Trastuzumab | Exatecan | None | Calc: 24507; | Calc: 53796; | 95%+ |
| (invention) | | glucuronide | | | | Obs: 24506 | Obs: 53795 | |
| | | trigger | | | | | | |
| ADC104 | DL4 | Triazole- | Trastuzumab | Exatecan | None | Calc: 24602; | Calc: 54081; | 95%+ |
| (comparative) | | linked | | | | Obs: 24601 | Obs: 54078 | |
| | | glucuronide | | | | | | |
| | | trigger | | | | | | |
| ADC105 | DL5 | Amide-linked | Trastuzumab | MMAE | Polysarcosine | Calc: 25750; | Calc: 57527; | 95%+ |
| (invention) | | glucuronide | | | 12 | Obs: 25750 | Obs: 57527 | |
| | | trigger | | | | | | |
| ADC106 | DL6 | Triazole- | Trastuzumab | MMAE | Polysarcosine | Calc: 25774; | Calc: 57599; | 95%+ |
| (comparative) | | linked | | | 12 | Obs: 25775 | Obs: 57601 | |
| | | glucuronide | | | | | | |
| | | trigger | | | | | | |
| ADC107 | DL7 | Amide-linked | Trastuzumab | MMAE | None | Calc: 24797; | Calc: 54667; | 95%+ |
| (invention) | | glucuronide | | | | Obs: 24796 | Obs: 54666 | |
| | | trigger | | | | | | |
| ADC108 | DL8 | Triazole- | Trastuzumab | MMAE | None | Calc: 24892; | Calc: 54952; | 95%+ |
| (comparative) | | linked | | | | Obs: 24891 | Obs: 54952 | |
| | | glucuronide | | | | | | |
| | | trigger | | | | | | |
| ADC109 | DL9 | Amide-linked | Trastuzumab | MMAE | None | Calc: 24809; | Calc: 54703; | 95%+ |
| (comparative) | | glucuronide | | | | Obs: 24808 | Obs: 54702 | |
| | | trigger | | | | | | |
| | | (amide | | | | | | |
| | | directly | | | | | | |
| | | linked to the | | | | | | |
| | | phenyl) | | | | | | |
| ADC110 | DL10 | Amide-linked | Trastuzumab | Exatecan | Polysarcosine | Calc: 25326; | Calc: 56255; | 95%+ |
| (invention) | | glucuronide | | | 10 | Obs: 25326 | Obs: 56253 | |
| | | trigger | | | | | | |
| ADC110-S | DL10-S | Amide-linked | Trastuzumab | Exatecan | Polysarcosine | Calc: 25326; | Calc: 56255; | 95%+ |
| (invention) | | glucuronide | | | 10 | Obs: 25326 | Obs: 56255 | |
| | | trigger | | | | | | |
| ADC110-R | DL10-R | Amide-linked | Trastuzumab | Exatecan | Polysarcosine | Calc: 25326; | Calc: 56255; | 95%+ |
| (invention) | | glucuronide | | | 10 | Obs: 25326 | Obs: 56254 | |
| | | trigger | | | | | | |
| ADC111 | DL10 | Amide-linked | Proprietary | Exatecan | Polysarcosine | Calc: 25885; | Calc: 56294; | 95%+ |
| (invention) | | glucuronide | mAb1 | | 10 | Obs: 25885 | Obs: 56294 | |
| | | trigger | | | | | | |
| ADC111-S | DL10-S | Amide-linked | Proprietary | Exatecan | Polysarcosine | Calc: 25885; | Calc: 56294; | 95%+ |
| (invention) | | glucuronide | mAb1 | | 10 | Obs: 25885 | Obs: 56293 | |
| | | trigger | | | | | | |
| ADC111-R | DL10-R | Amide-linked | Proprietary | Exatecan | Polysarcosine | Calc: 25885; | Calc: 56294; | 95%+ |
| (invention) | | glucuronide | mAb1 | | 10 | Obs: 25885 | Obs: 56292 | |
| | | trigger | | | | | | |
| ADC112 | DL11 | Triazole- | Trastuzumab | Exatecan | Polysarcosine | Calc: 25351; | Calc: 56330; | 95%+ |
| (invention) | | linked | | | 10 | Obs: 25350 | Obs: 56328 | |
| | | glucuronide | | | | | | |
| | | trigger | | | | | | |

Dipeptidase-based conjugates

| | | | | | | | | |
|---|---|---|---|---|---|---|---|---|
| ADC201 | DL12 | Amide-linked | Trastuzumab | Exatecan | Polysarcosine | Calc: 25401; | Calc: 56479; | 95%+ |
| (invention) | | dipeptide | | | 10 | Obs: 25401 | Obs: 56480 | |
| | | trigger | | | | | | |
| ADC202 | DL13 | Triazole- | Trastuzumab | Exatecan | Polysarcosine | Calc: 25425; | Calc: 56552; | 95%+ |
| (comparative) | | linked | | | 10 | Obs: 25425 | Obs: 56552 | |
| | | dipeptide | | | | | | |
| | | trigger | | | | | | |
| ADC203 | DL14 | Amide-linked | Proprietary | Exatecan | None | Calc: 25333; | Calc: 53629; | 95%+ |
| (invention) | | dipeptide | mAb2 | | | Obs: 25333 | Obs: 53629 | |
| | | trigger | | | | | | |
| ADC204 | DL15 | Triazole- | Proprietary | Exatecan | None | Calc: 25428; | Calc: 53914; | 95%+ |
| (comparative) | | linked | mAb2 | | | Obs: 25427 | Obs: 53914 | |
| | | dipeptide | | | | | | |
| | | trigger | | | | | | |
| ADC205 | DL16 | Amide-linked | Trastuzumab | MMAE | None | Calc: 24787; | Calc: 54637; | 95%+ |
| (invention) | | dipeptide | | | | Obs: 24786 | Obs: 54636 | |
| | | trigger | | | | | | |
| ADC206 | DL17 | Triazole- | Trastuzumab | MMAE | None | Calc: 24882; | Calc: 54922; | 95%+ |
| (comparative) | | linked | | | | Obs: 24882 | Obs: 54922 | |
| | | dipeptide | | | | | | |
| | | trigger | | | | | | |
| ADC207 | DL14 | Amide-linked | Trastuzumab | Exatecan | None | Calc: 24505; | Calc: 53790; | 95%+ |
| (invention) | | dipeptide | | | | Obs: 24504 | Obs: 53788 | |
| | | trigger | | | | | | |
| ADC208 | DL18 | Conventional | Trastuzumab | Exatecan | None | Calc: 24434; | Calc: 53577; | 71.2% |
| (comparative) | | linear | | | | Obs: 24433 | Obs: 53576 | (aggregated) |
| | | dipeptide | | | | | | |
| | | para- | | | | | | |
| | | aminobenzyl | | | | | | |
| | | spacer | | | | | | |
| ADC209 | DL19 | Conventional | Trastuzumab | MMAE | None | Calc: 24716; | Calc: 54424; | 95%+ but |
| (comparative) | | linear | | | | Obs: 24716 | Obs: 54423 | asymmetric |
| | | dipeptide | | | | | | tailing peak = |
| | | para- | | | | | | high |
| | | aminobenzyl | | | | | | hydrophobicity |
| | | spacer | | | | | | of the ADC |
| ADC210 | DL20 | Triazole- | Trastuzumab | Exatecan | Polysarcosine | Calc: 25341; | Calc: 56300; | 95%+ |
| (comparative) | | linked | | | 10 | Obs: 25341 | Obs: 56299 | |
| | | dipeptide | | | | | | |
| | | trigger | | | | | | |

### 3) Hydrophobic Interaction Chromatography (HIC) Profiles of Antibody-Drug Conjugates (ADC)

The apparent hydrophobicity of conjugates was assessed by hydrophobic interaction chromatography (HIC) on a Tosoh TSK-GEL BUTYL-NPR column, following the method described in section 2.

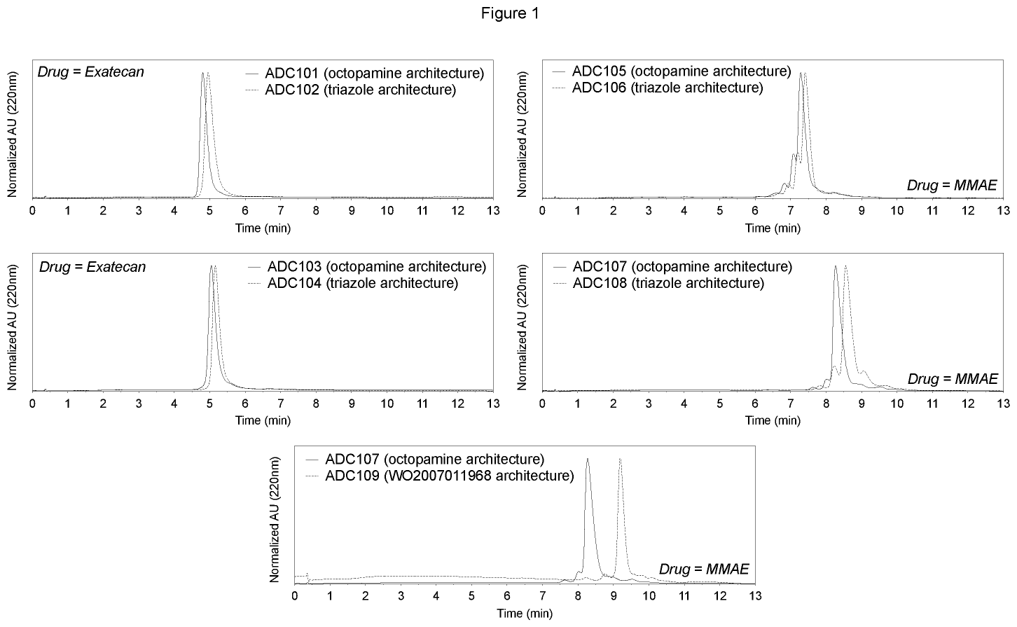

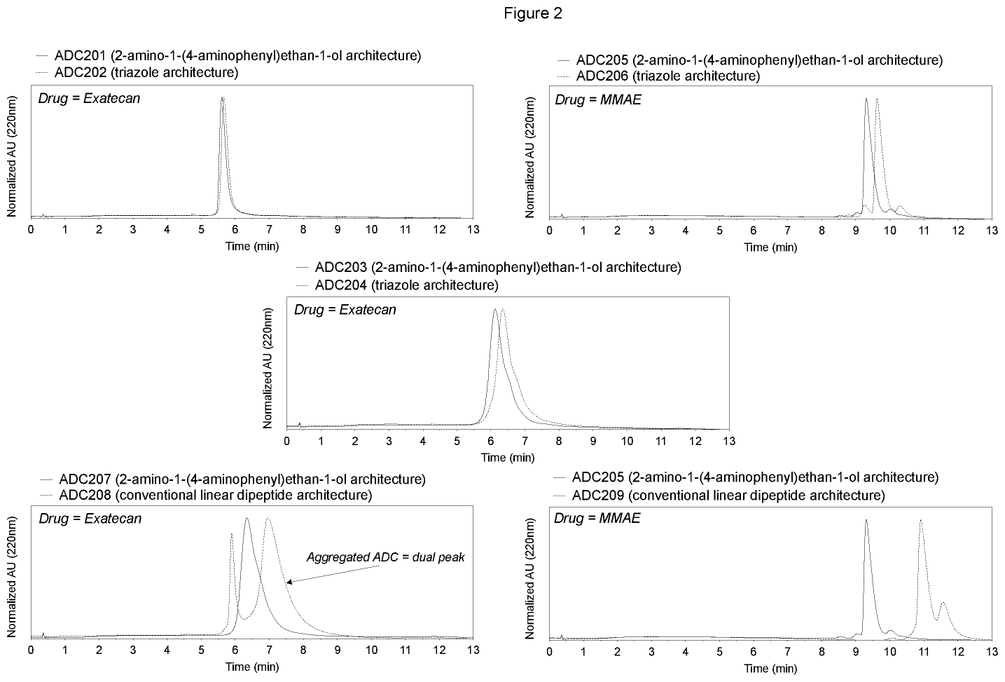

The results are shown in FIG. 1 for the glycosidase-based drug-linkers of the present invention (octopamine architecture) and in FIG. 2 for the dipeptidase-based drug-linkers of the present invention (2-amino-1-(4-aminophenyl)ethan-1-ol architecture). Conjugates of the present invention were systematically more hydrophilic (shorter retention time in the HIC chromatogram) when compared to known corresponding architectures. This effect is observed with glycosidase-based drug-linkers and dipeptidase-based drug-linkers. This effect is observed in the presence or absence of the hydrophobicity masking entity polysarcosine in the drug-linker architecture. This effect is observed with drug payloads of different nature and different levels of intrinsic hydrophobicity.

### 4) In Vitro Cytotoxicity Assays of Conjugates Based on Drug-Linkers of the Present Invention and Conjugates Based on Drug-Linkers of Known Architectures

In vitro cytotoxicity of conjugates was assessed on several antigen positive cancerous cell lines. Cells were plated in 96-well plates at an appropriate density depending on the cell line (between 1000 and 10 000 cells/well in 100 μL of appropriate culture media) and incubated at 37° C. for 24 hours. Serial dilutions of the tested compound previously dissolved in culture media (50 μL) were added, and incubation was carried at 37° C. out for 72 hours for MMAE-based conjugates and 144 hours for Exatecan-based conjugates. MTT (5 mg/mL, 20 μL, Sigma-Aldrich) was added into the wells, and incubation was continued for 1 to 2 hours at 37° C. Culture media was then carefully removed, and well content was homogeneously dissolved with acidified isopropanol. Absorbance values were measured on a Multiskan™ Sky microplate reader (Thermo Scientific) using a wavelength of 570 nm (with a reference wavelength of 690 nm). The IC50 concentration values compared to untreated control cells were determined using inhibition dose response curve fitting (GraphPad Prism 9).

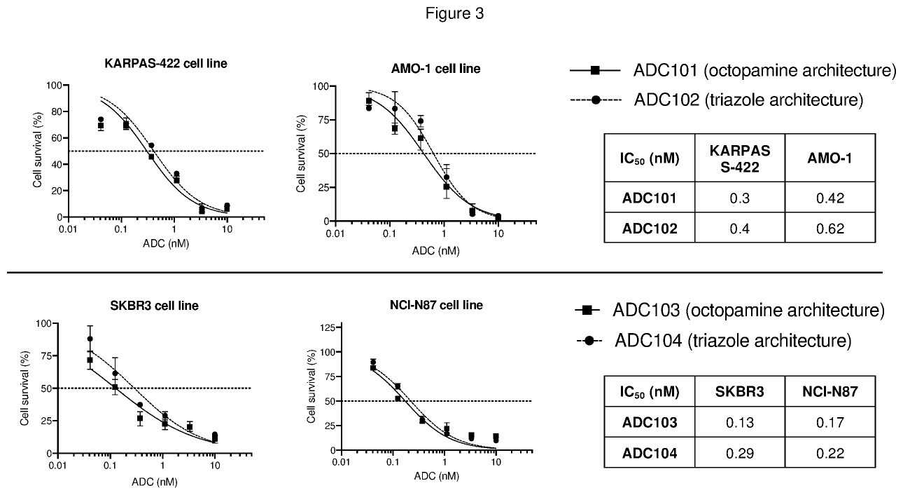

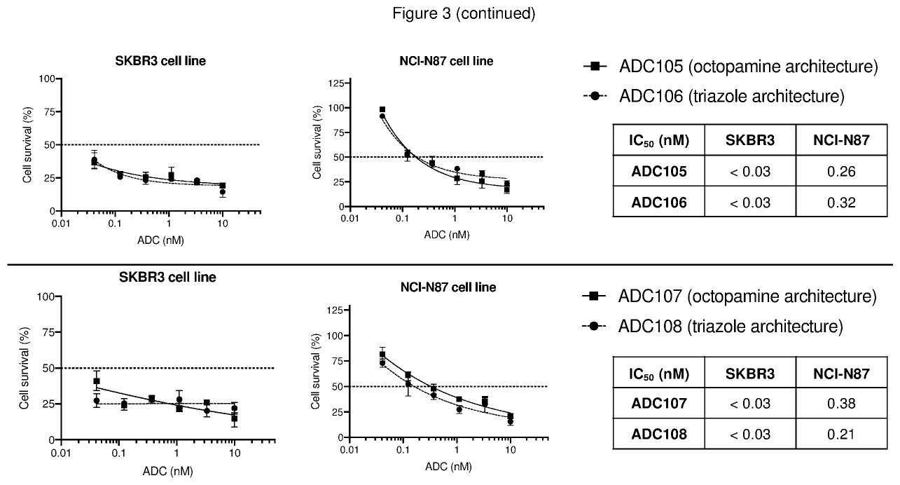

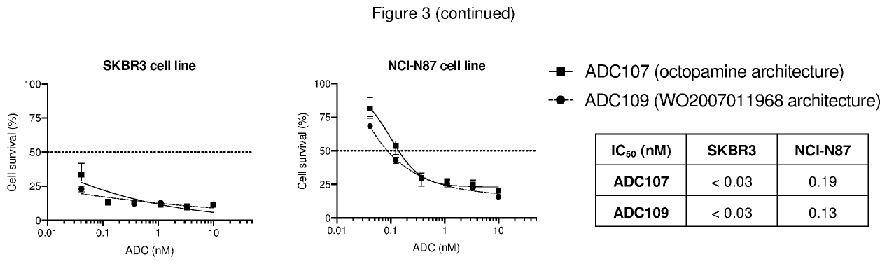

The results are shown in FIG. 3 for conjugates based on glycosidase-sensitive drug-linkers and in FIG. 4 for conjugates based on dipeptidase-sensitive drug-linkers. Conjugates of the present invention (octopamine and 2-amino-1-(4-aminophenyl)ethan-1-ol architectures) systematically showed similar potencies when compared to known corresponding architectures.

### 5) In Vitro Cytotoxicity Assays of Conjugates Based on DL10 Drug-Linker (Equimolar Diastereoisomeric Mixture) of the Present Invention, Stereo-Defined DL10-S Drug-Linker of the Present Invention and Stereo-Defined DL10-R Drug-Linker of the Present Invention

In vitro cytotoxicity of conjugates was assessed on several antigen positive cancerous cell lines, according to the experimental protocol described above in section 4).

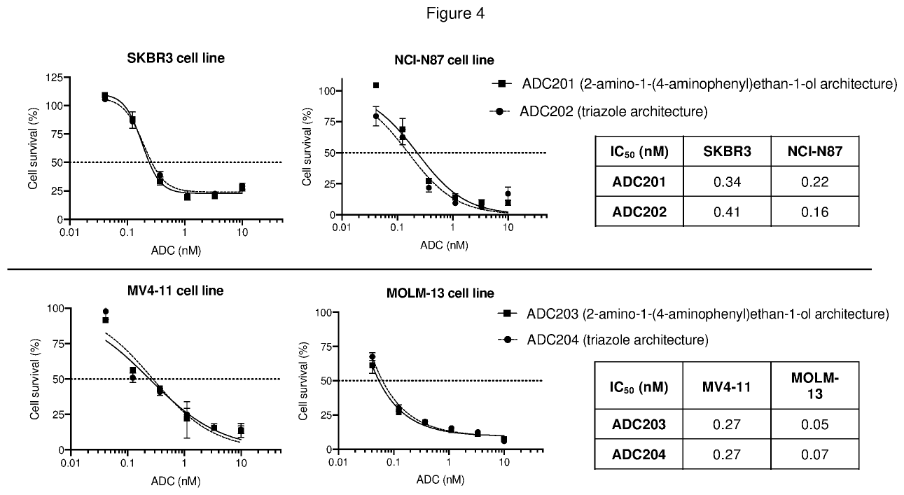

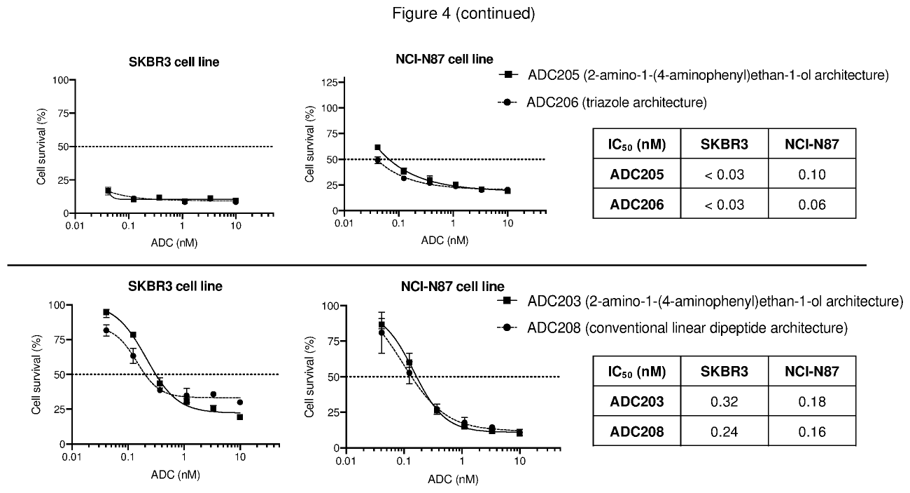

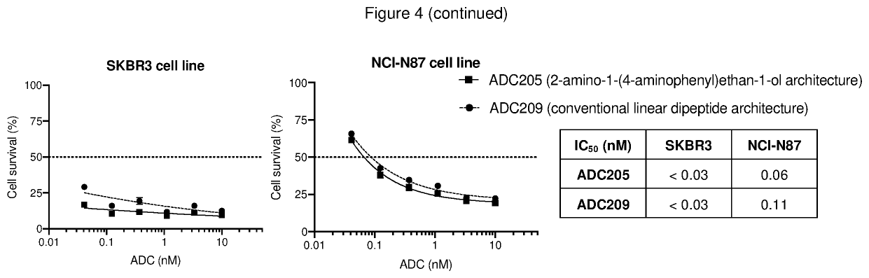

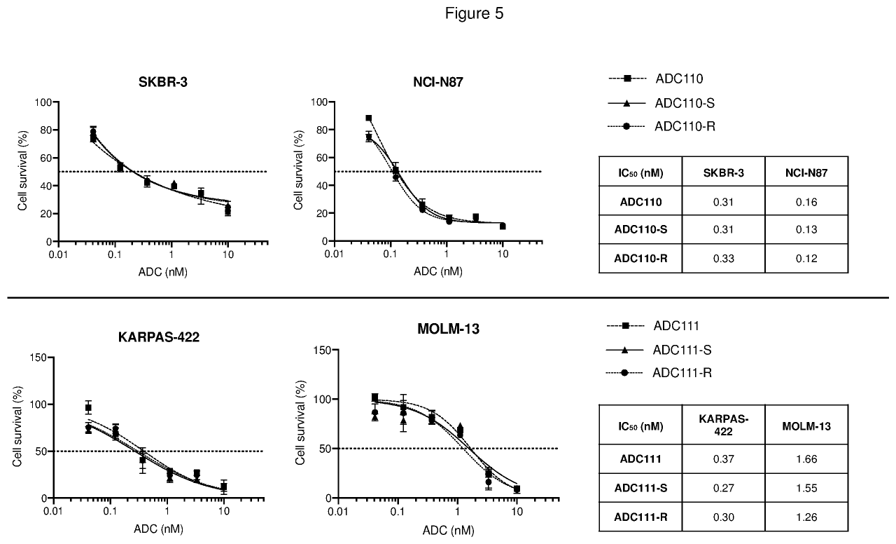

The results are shown in FIG. 5. No in vitro potency differences were observed between the stereopure drug-linkers and the equimolar diastereoisomeric mixture of the same drug-linker.

### 6) Pharmacokinetic Profile (Total Antibody-Drug Conjugate Concentration Over Time) in Rats Following a Single Intravenous 3 mg/kg Dose of Conjugate

ADCs were injected at 3 mg/kg in female Sprague-Dawley rats (4-6 weeks old—Charles River) via the tail vein (three animals per group, randomly assigned). Blood was drawn into citrate tubes via retro-orbital bleeding at various time points, processed to plasma and stored at −80° C. until analysis. ADC concentration was assessed using a human IgG ELISA kit (Stemcell™ Technologies) according to the manufacturer's protocol. Standard curves of corresponding monoclonal antibody were used for quantification. Pharmacokinetics parameters (clearance, half-life and AUC) were calculated by two-compartmental analysis using Microsoft® Excel® software incorporating PK functions (add-in developed by Usansky et al., Department of Pharmacokinetics and Drug Metabolism, Allergan, Irvine, USA).

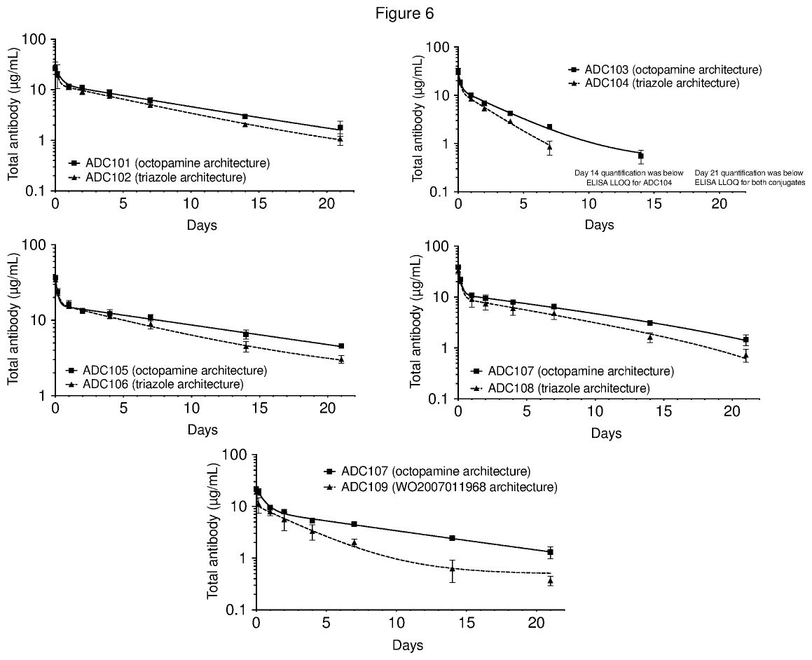

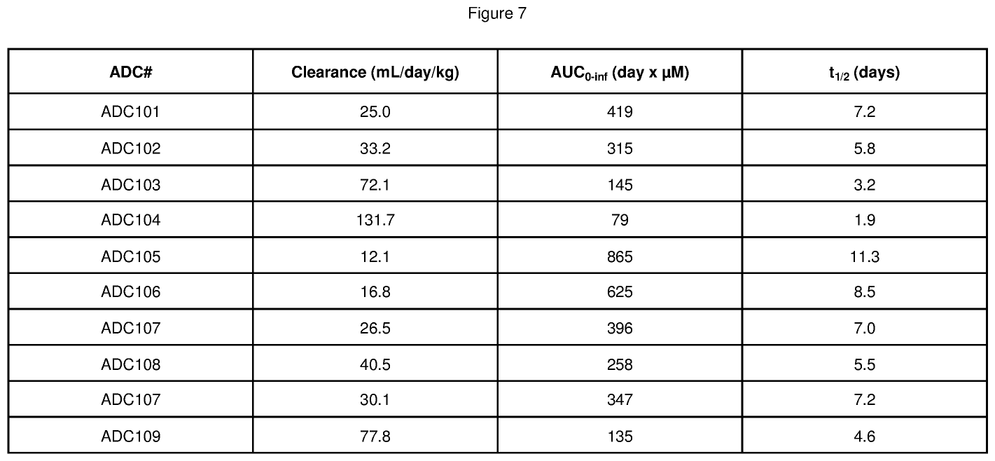

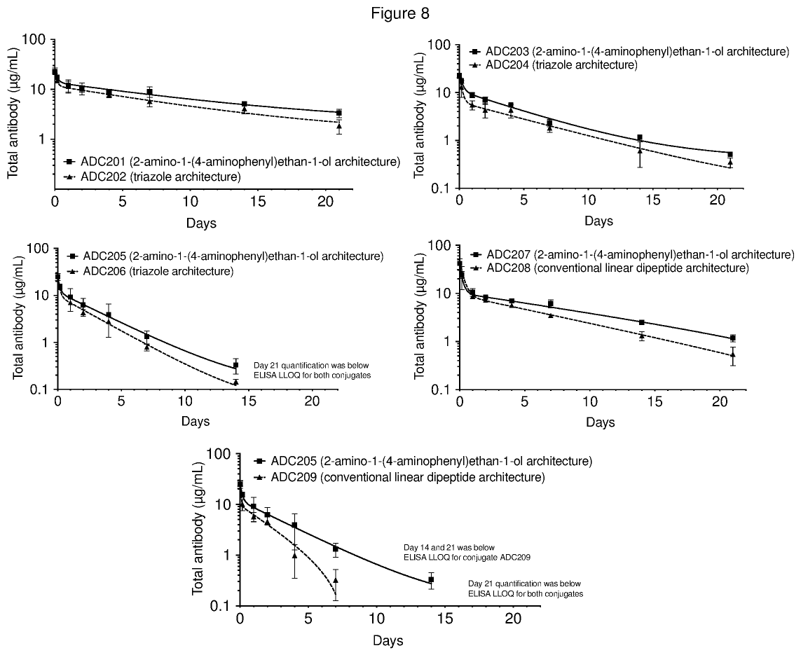

The results are shown in FIG. 6 (PK profiles) and FIG. 7 (PK parameters) for conjugates based on glycosidase-sensitive drug-linkers and in FIG. 8 (PK profiles) and FIG. 9 (PK parameters) for conjugates based on dipeptidase-sensitive drug-linkers. Conjugates of the present invention (octopamine and 2-amino-1-(4-aminophenyl)ethan-1-ol architectures) systematically yielded improved pharmacokinetic profiles and pharmacokinetic parameters (improved exposure, augmented half-life and decreased clearance rate) when compared to known corresponding architectures.

### 7) Tumor Volume (mm³) Over Time in a NCI-N87 HER2+ Gastric Cancer Xenograft Model Dosed Once Intravenously with 1 mg/kg of ADC110 Conjugate (Octopamine Architecture of the Invention with Glucuronide-Exatecan Payload) and ADC112 Conjugate (Triazole Architecture with Glucuronide-Exatecan Payload)

NCI-N₈₇ gastric cancer cells were implanted subcutaneously in female SCID mice (4 weeks old). ADCs were dosed once intravenously at a subcurative dose of 1 mg/kg when tumors had grown to approximately 150 mm³ (6 animals per group, assigned to minimize differences in initial tumor volumes between groups). Tumor volume was measured every 3-5 days by a caliper device and was calculated using the formula (L×W²)/2. Mice were sacrificed when the tumor volume exceeded 1000 mm³.

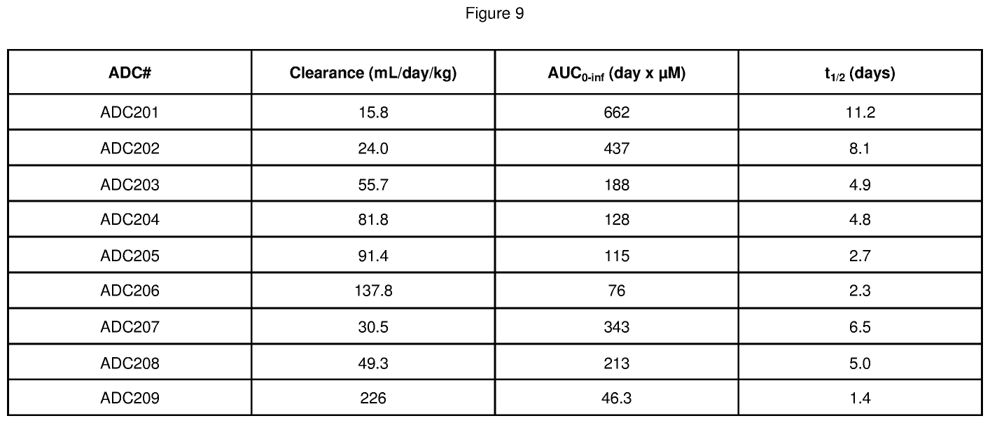

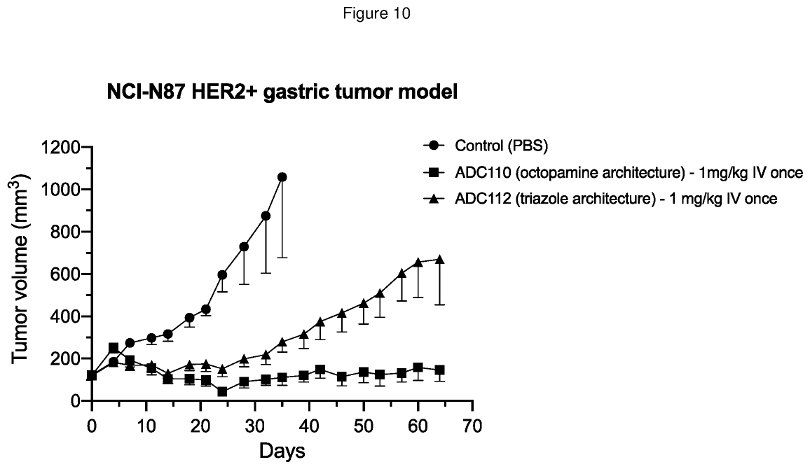

The results are shown in FIG. 10. Conjugate ADC110 based on the octopamine architecture of the invention showed improved in vivo activity when compared to conjugate ADC112 based on the triazole architecture. No significant body-weight loss was observed in all treated mice.

1.-17. (canceled)

18. A Ligand-Drug-Conjugate compound (LDC) having the following formula (I) 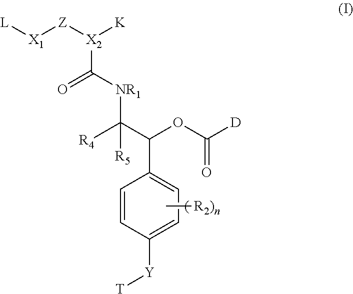
Wherein
L is a ligand;
X1 is a connector unit;
Z is an optional spacer;
X2 is a connector unit;
K is an optional hydrophobicity masking entity;
R1 is selected from the group consisting of H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof, said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR″—, —NR″—C(O)—O—, —O—C(O)NR″— and triazole;
D is an active agent;
each R2 is independently selected from the group consisting electron-withdrawing groups and C₁-C₄ alkyl;
n is 0, 1 or 2;
R4 is selected from the group consisting H, C₁-C₆ alkyl and C₂-C₆ alkenyl, said alkyl and alkenyl being optionally interrupted by one or more heteroatom or chemical groups selected from —O—, —S—, —C(O)—, and —NR″—;
R5 is selected from the group consisting H, C₁-C₆ alkyl and C₂-C₆ alkenyl, said alkyl and alkenyl being optionally interrupted by one or more heteroatom or chemical groups selected from —O—, —S—, —C(O)—, and —NR″—;
T is a sugar cleavable unit or a polypeptide cleavable unit;
Y is O when T is a sugar cleavable unit, or NR3 when T is a polypeptide cleavable unit;
R3 is selected from the group consisting of H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof, said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R′″ being independently selected from H and C₁-C₆ alkyl;
and pharmaceutically acceptable salts thereof.

19. LDC compound according to claim 18, wherein L is a ligand selected from the group consisting of polypeptides, proteins, antibodies and antibody fragments.

20. LDC compound according to claim 18, wherein D is selected from the group consisting of drugs.

21. LDC compound according to claim 18, wherein X1 and X2 are independently selected from the group consisting of one or more amino acid(s), one or more N-substituted amino acid, optionally substituted polyether, C₁-C₁₂ alkylene, arylene having 6 to 10 ring atoms, C₃-C₈ cycloalkylene, heterocycloalkylene having 5 to 10 ring atoms, heteroarylene having 5 to 10 ring atoms, C₂-C₁₀ alkenylene, and any combination thereof,
said alkylene and alkenylene being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole,
and said alkylene, arylene, cycloalkylene, heterocycloalkylene, heteroarylene, and alkenylene being optionally substituted with one or more of the substituents selected from: halogen, oxo, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R′″, —NR″—(CO)—R′, and —NR″R′″;
R′, R″ and R′″ being independently selected from H and C₁-C₆ alkyl.

22. LDC compound according to claim 18, wherein Z is independently selected from the group consisting of one or more amino acid(s), one or more N-substituted amino acid, optionally substituted polyether, C₁-C₁₂ alkylene, arylene having 6 to 10 ring atoms, C₃-C₈ cycloalkylene, heterocycloalkylene having 5 to 10 ring atoms, heteroarylene having 5 to 10 ring atoms, C₂-C₁₀ alkenylene, and any combination thereof,
said alkylene and alkenylene being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole,
and said alkylene, arylene, cycloalkylene, heterocycloalkylene, heteroarylene, and alkenylene being optionally substituted with one or more of the substituents selected from: halogen, oxo, —OH, —NO₂, —CN, C₁-C₆ alkyl, C₃-C₆ cycloalkyl, heterocyclyl having 5 to 10 ring atoms, aryl having 6 to 10 ring atoms, heteroaryl having 5 to 10 ring atoms, C₁-C₆ alkoxy, C₁-C₆ haloalkyl, C₁-C₆ haloalkoxy, —(CO)—R′, —O—(CO)—R′, —(CO)—O—R′, —(CO)—NR″R′″, —NR″—(CO)—R′, and —NR″R′″;
R′, R″ and R′″ being independently selected from H and C₁-C₆ alkyl.

23. LDC compound according to claim 18, wherein K is a polysarcosine.

24. LDC compound according to claim 18, wherein T is a sugar cleavable unit which is a glucuronide.

25. LDC compound according to claim 18, wherein T is a dipeptide.

26. LDC compound according to claim 18, wherein the compound is a compound of formula (VI) 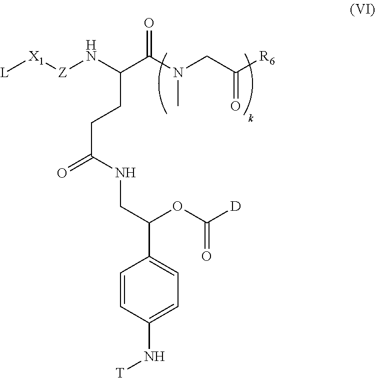
wherein k is an integer between 2 and 50; and T is a polypeptide cleavable unit.

27. LDC compound according to claim 18, wherein the compound is a compound of formula (VIII) 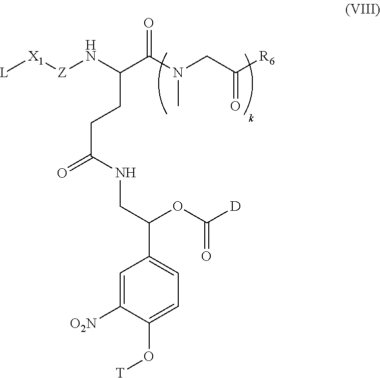
wherein k is an integer between 2 and 50; and T is a sugar cleavable unit.

28. A pharmaceutical composition comprising a LDC compound according to claim 18, and a pharmaceutically acceptable carrier.

29. A method for treating cancer, inflammatory diseases or infectious diseases, said method comprising administering to a subject in need thereof, a therapeutically efficient amount of
a LDC compound according to claim 18.

30. An intermediate compound of formula (II) 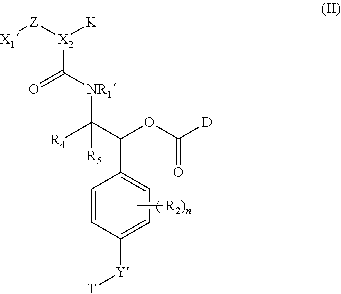
Wherein
X1′ is a group which can react with a ligand to form a connector unit;
Z is an optional spacer;
X2 is a connector unit;
K is an optional hydrophobicity masking entity;
R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
D is an active agent;
each R2 is independently selected from the group consisting electron-withdrawing groups and C₁-C₄ alkyl;
n is 0, 1 or 2;
R4 is selected from the group consisting H, C₁-C₆ alkyl and C₂-C₆ alkenyl, said alkyl and alkenyl being optionally interrupted by one or more heteroatom or chemical groups selected from —O—, —S—, —C(O)—, and —NR″—;
R5 is selected from the group consisting H, C₁-C₆ alkyl and C₂-C₆ alkenyl, said alkyl and alkenyl being optionally interrupted by one or more heteroatom or chemical groups selected from —O—, —S—, —C(O)—, and —NR″—;
T is a sugar cleavable unit or a polypeptide cleavable unit;
Y′ is O when T is a sugar cleavable unit, or NR3′ when T is a polypeptide cleavable unit;
R3′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R″ being independently selected from H and C₁-C₆ alkyl;
and pharmaceutically acceptable salts thereof.

31. Intermediate compound of formula (II) according to claim 28, wherein K is a polysarcosine.

32. An intermediate compound of formula (III) 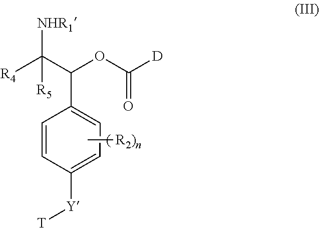
Wherein
R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
D is an active agent;
each R2 is independently selected from the group consisting electron-withdrawing groups and C₁-C₄ alkyl;
n is 0, 1 or 2;
R4 is selected from the group consisting H, C₁-C₆ alkyl and C₂-C₆ alkenyl, said alkyl and alkenyl being optionally interrupted by one or more heteroatom or chemical groups selected from —O—, —S—, —C(O)—, and —NR″—;
R5 is selected from the group consisting H, C₁-C₆ alkyl and C₂-C₆ alkenyl, said alkyl and alkenyl being optionally interrupted by one or more heteroatom or chemical groups selected from —O—, —S—, —C(O)—, and —NR″—;
T is a sugar cleavable unit or a polypeptide cleavable unit;
Y′ is O when T is a sugar cleavable unit, or NR3′ when T is a polypeptide cleavable unit;
R3′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R′″ being independently selected from H and C₁-C₆ alkyl;
and pharmaceutically acceptable salts thereof.

33. An intermediate compound of formula (IV) 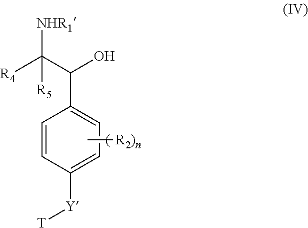
Wherein
R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
each R2 is independently selected from the group consisting electron-withdrawing groups and C₁-C₄ alkyl;
n is 0, 1 or 2;
R4 is H;
R5 is H;
T is a sugar cleavable unit;
Y′ is O;
and pharmaceutically acceptable salts thereof.

34. An intermediate compound of formula (IV) 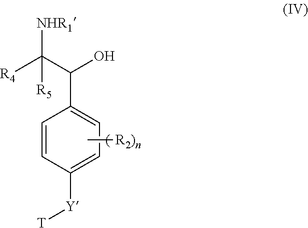
Wherein
R1′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
each R2 is independently selected from the group consisting electron-withdrawing groups and C₁-C₄ alkyl;
n is 0, 1 or 2;
R4 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
R5 is selected from the group consisting of H, C₁-C₆ alkyl and C₂-C₆ alkenyl, said alkyl and alkenyl being optionally interrupted by one or more heteroatoms or chemical groups selected from —O—, —S—, —C(O)—, and —NR″—;
Y′ is NR3′;
T is a polypeptide cleavable unit;
R3′ is selected from the group consisting of amino protecting groups, H, C₁-C₂₄ alkyl, C₂-C₆ alkenyl; optionally substituted polyether, aryl having 6 to 10 ring atoms, C₃-C₈ cycloalkyl, heterocycloalkyl having 3 to 10 ring atoms, heteroaryl having 5 to 10 ring atoms, and any combination thereof,
said alkyl and alkenyl being optionally interrupted by one or more heteroatom or chemical groups selected from —O—, —S—, —C(O)—, —NR″—, —C(O)NR″—, —NR″—C(O)—, —NR″—C(O)—NR′″—, —NR″—C(O)—O—, —O—C(O)NR″— and triazole;
R″ and R′″ being independently selected from H and C₁-C₆ alkyl;
and pharmaceutically acceptable salts thereof.

35. LDC compound according to claim 18, wherein L is a ligand selected from the group consisting of antibodies and antibody fragments.

36. LDC compound according to claim 18, wherein D is an anticancer drug.

37. LDC compound according to claim 18, wherein T is a dipeptide selected from Val-Cit, Val-Ala and Phe-Lys.

38. LDC compound according to claim 18, wherein
L is a ligand selected from the group consisting of antibodies and antibody fragments,
D is an anticancer drug,
T is a dipeptide selected from Val-Cit, Val-Ala and Phe-Lys, and
K is a polysarcosine.

39. A method for treating cancer, inflammatory diseases or infectious diseases, said method comprising administering to a subject in need thereof, a therapeutically efficient amount of
a pharmaceutical composition according to claim 28.
